(19) United States
(12) Patent Application Publication
(10) Pub. No.: US 2023/0193248 A1
(43) Pub. Date: Jun. 22, 2023

(54) METHODS FOR PROTEIN IDENTIFICATION BASED ON ENCODING REACTIONS

(71) Applicant: Encodia, Inc., San Diego, CA (US)

(72) Inventors: Sean SUASTEGUI, San Diego, CA (US); Soumya GANGULY, San Diego, CA (US); Stephen VERESPY, III, San Diego, CA (US); Eric OKERBERG, San Diego, CA (US); Andrew SAWAYAMA, San Diego, CA (US)

(73) Assignee: Encodia, Inc., San Diego, CA (US)

(21) Appl. No.: 18/065,282

(22) Filed: Dec. 13, 2022

Related U.S. Application Data
(60) Provisional application No. 63/290,327, filed on Dec. 16, 2021.

(51) Int. Cl. C12N 15/10; C12Q 1/6869; C40B 20/04; C40B 70/00

(52) U.S. Cl. CPC C12N 15/1065; C12Q 1/6869; C40B 20/04; C40B 70/00

(57) ABSTRACT

The present disclosure relates to methods and kits for high-throughput, highly parallel polypeptide identification employing labeling of specific amino acid residues, barcoding and nucleic acid encoding of the labeled residues. The workflow and architecture described herein allow identification of polypeptides in a sample in a cyclic manner based on encoding of their specific amino acid residues and without use of protein-based or aptamer-based binding agents. Successful “binder-free” encoding takes advantage of the high affinity and specificity of nucleic acid tags, as well as the specific chemistry of certain amino acid side chains.

### CROSS-REFERENCE TO RELATED APPLICATION

This application claims priority to U.S. Provisional Patent Application No. 63/290,327 filed Dec. 16, 2021, entitled “METHODS FOR PROTEIN IDENTIFICATION BASED ON ENCODING REACTIONS,” which is herein incorporated by reference in its entirety for all purposes.

### REFERENCE TO AN ELECTRONIC SEQUENCE LISTING

The contents of the electronic sequence listing (2003700SEQLIST.xml; Size: 23,115 bytes; and Date of Creation: Dec. 12, 2022) is herein incorporated by reference in its entirety.

### TECHNICAL FIELD

This disclosure generally relates to biotechnology, and in particular to analysis of polypeptide(s) in a cyclic manner employing labeling of specific amino acid residues, barcoding and nucleic acid encoding of the labeled residues in order to identify polypeptide(s) in a highly parallel manner. The disclosure finds utility at least in a variety of methods and related kits for high-throughput polypeptides identification.

### BACKGROUND

High-throughput nucleic acid sequencing has transformed life science research through improved sensitivity and lower costs, and consequently has found multiple applications in medicine and personal genomics. Similar high-throughput approaches to protein identification are not currently available, yet knowledge about protein identity in a sample can be crucial for better understanding of proteome dynamics in health and disease. Highly-parallel identification of proteins is challenging for several reasons. The use of affinity-based assays is often difficult due to several key challenges. One significant challenge is multiplexing the readout of a collection of affinity agents to a collection of cognate polypeptides; another challenge is minimizing cross-reactivity between the affinity agents and off-target polypeptides; a third challenge is developing an efficient high-throughput read-out platform.

Recently, methods have been disclosed that utilize use of binding agents for high-throughput polypeptide identification and sequencing, for example, U.S. Pat. No. 9,435,810 B2, U.S. Pat. No. 10,473,654 B1, U.S. Pat. No. 9,625,469 B2, U.S. Pat. No. 10,006,917 B2, and the following published patent applications: WO2010065531A1, US 20190145982 A1, US 20200217853 A1, US 20200348308 A1, US 20200400677 A1, US 20200217853 A1, and US 20180299460 A1. Some of these methods utilize N-terminal amino acid (NTAA) recognition by binding agents as a critical step in a polypeptide identification and sequencing assay. A number of methods to evolve specific NTAA binders from different scaffolds for recognizing a particular terminal amino acid have also been proposed, including directed evolution approaches to derive amino acyl tRNA synthetases, N-recognins such as ClpS and ClpS2, anticalins, and aminopeptidases, which are disclosed, for example, in U.S. Pat. No. 9,435,810 B2 and US patent publication 2019/0145982 A1. However, identifying binding agents that afford amino acid specificity with sufficiently strong affinity has proven challenging. Binding affinity and/or specificity towards a terminal amino acid residue (P1) can vary depending on neighboring amino acid residues of the polypeptide to be analyzed, e.g., the penultimate terminal amino acid residue (P2) and the antepenultimate amino acid residue (P3). It may be preferred that binding agents and detection assays are performed in a manner that allows specificity and stability in a controllable manner that allows processing of a plurality of binding agents and polypeptides at the same time. However, current reagents and methods are somewhat limited in these aspects. Accordingly, there remains a need for improved techniques relating to polypeptides identification in a sample.

The present disclosure describes novel and improved approaches for performing highly-parallel identification of polypeptide(s) by utilizing labeling of specific amino acid residues, barcoding and nucleic acid encoding of the labeled residues. These approaches address a need for proteomics technology that is highly-parallelized, accurate, sensitive, and/or high-throughput. These and other aspects of the disclosure will be apparent upon reference to the following detailed description. To this end, various references are set forth herein which describe in more detail certain background information, procedures, compounds and/or compositions, and are each hereby incorporated by reference in their entireties.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

Several variants of the ProteoCode™ assay that allow for high-throughput polypeptide characterization and identification have been disclosed in the following published patent applications: US 2019/0145982 A1, US 2020/0348308 A1, US 2020/0348307 A1 and US 2021/0208150 A1. During an exemplary assay, an immobilized polypeptide associated with a nucleic acid recording tag is contacted with binding agents capable of binding to the polypeptide, wherein each binding agent comprises a nucleic acid coding tag with identifying information regarding the binding agent. During each binding cycle, the coding tag and the recording tag are located in a sufficient proximity for interaction, and the information regarding the binding agent that bound to the polypeptide at this cycle is transferred from the coding tag to the recording tag, thus generating an extended recording tag.

After two or more successive binding cycles, a nucleic acid encoded library representative of the binding history of the macromolecule is generated and encoded in the extended recording tag. Following the analysis of the extended recording tag (usually by a nucleic acid sequencing method), information about the binding agents bound to the polypeptide at each cycle can be decoded, providing information regarding components of the polypeptide to which the binding agents were bound. Thus, the ProteoCode™ assay represents an unconventional way of characterizing, identifying or quantifying the polypeptide's components, and is suitable for highly-parallel, high-throughput polypeptide characterization, such as polypeptide identification and/or de novo sequencing.

Provided herein is an alternative workflow and architecture for high-throughput polypeptide identification assay that allows identification of polypeptide(s) in a sample without protein-based or aptamer-based binding agents (referred herein as binder-free encoding methods). The disclosed methods utilize barcoded nucleic acid tags to selectively label specific amino acid residue(s) of an immobilized polypeptide to be identified. Each nucleic acid tag is configured to selectively label a single type of amino acid residue, or a few types of amino acid residues. For example, some nucleic acid tags can be designed to selectively label both glutamate and aspartate residues. Information regarding the specific amino acid residues are kept in the tag barcodes and can be transferred in a cyclic manner to a recording tag associated with the immobilized polypeptide. Finally, the sequence of the recording tag extended after the information transfer can be analyzed, and information regarding the specific amino acid residue(s) of the polypeptide and their position(s) can be decoded bioinformatically, followed by polypeptide identification based on matching of the obtained polypeptide signature with corresponding signatures of polypeptide(s) that may presumable be present in the sample. Such signatures may be generated by extracting polypeptide information from a genomic or proteomic database.

Successful “binder-free” encoding takes advantage of the high affinity and specificity of nucleic acid tags, as well as the specific chemistry of certain amino acid side chains. Utilizing known methods of reacting and labeling amino acid side chains in polypeptides, it is possible to add unique coding tag sequences directly onto the side chains of specific amino acid residues. Sample preparation for the proposed polypeptide identification workflow includes digesting proteins into polypeptides followed by associating the polypeptides with a nucleic acid recording tag on a solid support, such as, for example, a porous bead.

Thus, one embodiment of this disclosure provides a method for identifying a polypeptide, the method comprising the steps of: (a) providing the polypeptide and an associated recording tag joined to a solid support; (b) contacting the polypeptide with a plurality of coding tags, wherein each coding tag of the plurality of coding tags is configured to react selectively with a specific type of amino acid residue(s) and comprises a barcode region with identifying information regarding the specific type of amino acid residue(s) to which the coding tag reacts selectively, thereby obtaining the polypeptide comprising coding tags attached to the specific amino acid residues; (c) contacting the polypeptide comprising coding tags attached to the specific amino acid residues with a plurality of complementary coding tags, wherein each complementary coding tag of the plurality of complementary coding tags comprises (i) a region complementary to the barcode region of a corresponding coding tag, (ii) a first spacer region complementary to a first complementary spacer region of the recording tag, and (iii) a moiety configured, when in a close proximity, to be covalently coupled to the NTAA of the polypeptide, or to a modified NTAA of the polypeptide; (d) providing conditions for covalently coupling the moiety of complementary coding tags to the NTAA of the polypeptide or the modified NTAA of the polypeptide; (e) removing complementary coding tags that are not covalently coupled to the NTAA of the polypeptide; (f) transferring identifying information of the barcode region or the region complementary to the barcode region from the complementary coding tag covalently coupled to the NTAA of the polypeptide to the recording tag, wherein transferring the identifying information comprises a primer extension or ligation; (g) removing the NTAA of the polypeptide, thereby exposing a new NTAA; (h) adding a second order complementary spacer region to the recording tag extended at step (f); (j) repeating steps (c)-(h) one or more times by replacing at step (c) the first spacer region of the complementary coding tags with a second or higher order spacer region complementary to the second or higher order complementary spacer region of the recording tag, and by replacing at step (h) the second complementary spacer region with a third or higher order complementary spacer region; and (k) analyzing the recording tag extended at step (i) by a nucleic acid sequencing method, and obtaining information regarding the specific amino acid residues of the polypeptide; thereby identifying the polypeptide.

Another embodiment provided herein is a method for identifying a polypeptide, the method comprising the steps of: (a) providing the polypeptide and an associated recording tag joined to a solid support; (b) contacting the polypeptide with a plurality of coding tags, wherein each coding tag of the plurality of coding tags is configured to react selectively with a specific type of amino acid residue(s) and comprises: i) a barcode region with identifying information regarding the specific type of amino acid residue(s) to which the coding tag reacts selectively, ii) an identification region unique for each specific type of amino acid residue(s) to which the coding tags react selectively, and iii) a recognition region for a site-specific restriction enzyme, located between the barcode region and the identification region, thereby obtaining the polypeptide comprising coding tags attached to the specific amino acid residues; (c) providing conditions for hybridization of the recording tag to one of the coding tags attached to the specific amino acid residue(s), thereby forming a double stranded region, and extending the recording tag (using the coding tag as a template), thereby transferring information of the barcode region and the recognition region from the coding tag to the recording tag; (d) cutting the recognition region by providing the site-specific restriction enzyme, so that the extended recording tag is released and only a coding tag stub comprising the identification region of the coding tag remains attached to the polypeptide; (e) repeating steps (c) and (d) for all other coding tags attached to the specific amino acid residues of the polypeptide, thereby obtaining the polypeptide comprising coding tag stubs attached to the specific amino acid residues; (f) removing the NTAA of the polypeptide, thereby exposing a new NTAA; (g) adding a second order complementary spacer region to the recording tag extended at step (f); (h) restoring coding tags from the coding tag stubs using the identification region; (j) repeating steps (c)-(h) one or more times; and (k) analyzing the recording tag extended at step (j) by a nucleic acid sequencing method, and obtaining information regarding the specific amino acid residues of the polypeptide before and after removing step, thereby identifying the polypeptide.

### BRIEF DESCRIPTION OF THE DRAWINGS

Non-limiting embodiments of the present invention will be described by way of example with reference to the accompanying figures, which are schematic and are not intended to be drawn to scale. For purposes of illustration, not every component is labeled in every figure, nor is every component of each embodiment of the invention shown where illustration is not necessary to allow those of ordinary skill in the art to understand the invention.

FIG. 1. Exemplary ProteoCode peptide sequencing assay with native NTAA binders. (1) Peptide molecules are each labeled with a DNA recording tag and attached to beads at a low molecular density, a sparsity that permits only intramolecular information transfer to occur. (2) Next, an NTAA binding agent labelled with a DNA coding tag binds to the native NTAA residue. After binding and washing, the coding tag information is transferred enzymatically to the recording tag (via extension or ligation). (3) Next, the peptide N-terminal amino acid (NTAA) is labeled with an N-terminal modification and removed by using mild Edman-like elimination chemistry or by a Cleavase enzyme. After n cycles, a DNA library element representing then amino acids of the peptide sequence is formed and can be sequenced by NGS. A representative structure of an NGS library element after 7 cycles is shown.

FIG. 2A-2B depict exemplary structures of reactive alkyne probes (electrophiles) capable of specifically targeting specific types of amino acid residues in polypeptides. Examples of such reactive alkyne probes include: IA-alkyne and EBX2-alkyne for cysteines; STP-alkyne, ArSq-alkyne and EBA-alkyne for lysines; PTAD-alkyne and SuTEx2-alkyne for tyrosines; HC-alkyne, MeTet-alkyne and Az-alkyne for aspartates and glutamates; OxMet2-alkyne for methionines; CP-alkyne, HMN-alkyne, MMP-alkyne for tryptophans; CP-alkyne for histidines; and PhGO-alkyne for arginines.

FIG. 3 shows exemplary modifying agents that can be used to generate modified amino acid residues of a polypeptide. The NHS ester group reacts with lysine residues of the polypeptide, and TCO is the first reactive handle.

FIG. 4 shows exemplary sample preparation procedure that comprises protein denaturation and digestion, labeling Lys and NTAA residues.

FIG. 5 shows continuation of the exemplary sample preparation procedure that comprises functionalization of Arg residues, formation of peptide-DNA conjugates and cleavage of the N-terminal linker.

FIG. 6 shows exemplary immobilization of peptide-DNA conjugates on a solid support congaing a Recording Tag (RT), followed by attachment of coding tags (CTs) configured to react selectively with a specific type of amino acid residue(s) of the immobilized polypeptide (shown for internal side chains of C, K, D/E, W, M, Y residues).

FIG. 7 shows exemplary N-terminal functionalization of the immobilized polypeptide with alkyne, followed by hybridization of CTs with complementary coding tags having a functional reactive group (azide).

FIG. 8 shows exemplary initiating covalent coupling of the moiety of the complementary coding tag to the modified NTAA of the polypeptide, followed by washing away non-coupled complementary coding tags, and hybridization between RT and the complementary coding tag through spacer regions.

FIG. 9 shows exemplary RT splint ligation, followed by enzymatic or chemical cleavage of the NTAA of the immobilized polypeptide.

FIG. 10 shows exemplary continuation of the encoding method depicted in FIG. 6-FIG. 9, which comprises repeating encoding cycles (n) times, followed by optionally cleaving and/or amplifying the RT extended after the previous encoding cycles, and then sequencing the extended RT.

FIG. 11 shows an exemplary alternative embodiment for binder-free encoding, which does not include using the reactive moiety configured to be covalently coupled to the NTAA of the immobilized polypeptide.

FIG. 12 shows selected steps of the exemplary alternative embodiment shown in FIG. 11.

FIG. 13 shows selected steps of the exemplary alternative embodiment shown in FIG. 11.

FIG. 14A illustrates exemplary cleavages of M15-L-modified NTAAs of a model polypeptide (M15-L-P1-AR) by engineered dipeptidyl peptidase enzymes. A compilation of seven different modified dipeptidyl peptidase clones was used to generate the spectrum of cleavage profiles across all 20 M15-L-modified NTAAs as shown. Data were generated by HPLC analysis (UV absorbance) of cleaved versus intact peptides after the cleavase assay. FIG. 14B shows cleavage events on peptides attached to DNA tag using gel-shift analysis on an SDS-PAGE gel.

FIG. 15A-FIG. 15B show the results of an exemplary cleavage reaction to evaluate activity of an engineered dipeptidyl peptidase mutant on a NTM-modified peptide (M15-K(biotin) attached to an AAR peptide). A designates a signal from M15-K(biotin)-AAR peptide; B designates a signal from M15-K(biotin)-A molecule, and C designates a signal from a control peptide. The UV absorbance of both starting material (FIG. 15A) and reaction product (FIG. 15B) was measured on HPLC.

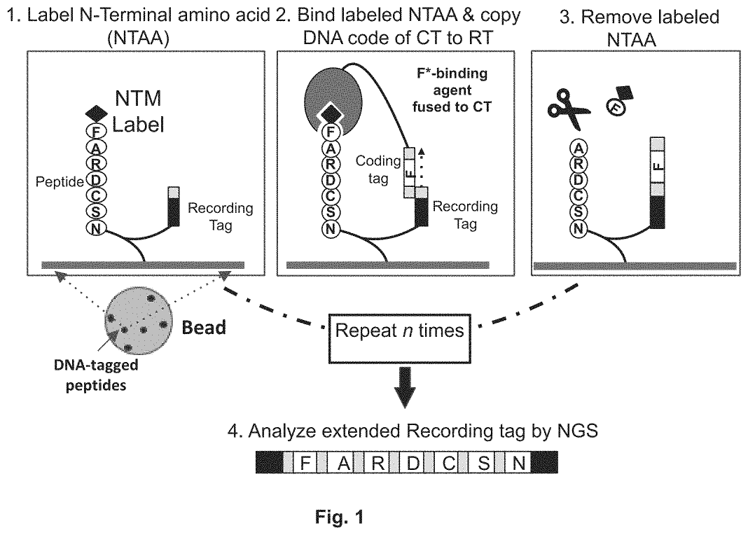

### DETAILED DESCRIPTION

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured. All publications, including patent documents, scientific articles and databases, referred to in this application are incorporated by reference in their entireties for all purposes. Citation of the publications or documents is not intended as an admission that any of them is pertinent prior art, nor does it constitute any admission as to the contents or date of these publications or documents.

### DEFINITIONS

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an” and “the” include plural referents unless the context clearly dictates otherwise. Thus, for example, reference to “a peptide” includes one or more peptides, or mixtures of peptides. Also, and unless specifically stated or obvious from context, as used herein, the term “or” is understood to be inclusive and covers both “or” and “and”.

As used herein, the term “sample” refers to anything which may contain an analyte for which an analyte assay is desired. As used herein, a “sample” can be a solution, a suspension, liquid, powder, a paste, aqueous, non-aqueous or any combination thereof. In some embodiments, the sample is a biological sample. A biological sample of the present disclosure encompasses a sample in the form of a solution, a suspension, a liquid, a powder, a paste, an aqueous sample, or a non-aqueous sample. As used herein, a “biological sample” includes any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. In some embodiments, the sample can be derived from a tissue or a body fluid, for example, a connective, epithelium, muscle or nerve tissue; a tissue selected from the group consisting of brain, lung, liver, spleen, bone marrow, thymus, heart, lymph, blood, bone, cartilage, pancreas, kidney, gall bladder, stomach, intestine, testis, ovary, uterus, rectum, nervous system, gland, and internal blood vessels; or a body fluid selected from the group consisting of blood, urine, saliva, bone marrow, sperm, an ascitic fluid, and subfractions thereof, e.g., serum or plasma.

As used herein, the term “polypeptide” encompasses peptides and proteins, and refers to a molecule comprising a chain of two or more amino acids joined by peptide bonds. In some embodiments, a polypeptide comprises 2 to 50 amino acids. In some embodiments, a polypeptide does not comprise a secondary, tertiary, or higher structure. In some embodiments, the polypeptide is a protein. In some embodiments, a protein comprises 30 or more amino acids. In some embodiments, in addition to a primary structure, a protein comprises a secondary, tertiary, or higher structure. The amino acids of the polypeptides are most typically L-amino acids, but may also be D-amino acids, modified amino acids, amino acid analogs, amino acid mimetics, or any combination thereof. Polypeptides may be naturally occurring, synthetically produced, or recombinantly expressed. Polypeptides may be synthetically produced, isolated, recombinantly expressed, or be produced by a combination of methodologies as described above. Polypeptides may also comprise additional groups modifying the amino acid chain, for example, functional groups added via post-translational modification. The polymer may be linear or branched, it may comprise modified amino acids, and it may be interrupted by non-amino acids. The term also encompasses an amino acid polymer that has been modified naturally or by intervention; for example, disulfide bond formation, glycosylation, lipidation, acetylation, phosphorylation, or any other manipulation or modification, such as conjugation with a labeling component.

As used herein, the term “amino acid” refers to an organic compound comprising an amine group, a carboxylic acid group, and a side-chain specific to each amino acid, which serve as a monomeric subunit of a peptide. An amino acid includes the 20 standard, naturally occurring or canonical amino acids as well as non-standard amino acids. The standard, naturally-occurring (or natural) types of amino acids include Alanine (A or Ala), Cysteine (C or Cys), Aspartic Acid (D or Asp), Glutamic Acid (E or Glu), Phenylalanine (F or Phe), Glycine (G or Gly), Histidine (H or His), Isoleucine (I or Ile), Lysine (K or Lys), Leucine (L or Leu), Methionine (M or Met), Asparagine (N or Asn), Proline (P or Pro), Glutamine (Q or Gln), Arginine (R or Arg), Serine (S or Ser), Threonine (T or Thr), Valine (V or Val), Tryptophan (W or Trp), and Tyrosine (Y or Tyr). These 20 amino acids form 20 specific types of amino acid residues present in polypeptides. An amino acid may be an L-amino acid or a D-amino acid. Non-standard amino acids may be modified amino acids, amino acid analogs, amino acid mimetics, non-standard proteinogenic amino acids, or non-proteinogenic amino acids that occur naturally or are chemically synthesized. Examples of non-standard amino acids include, but are not limited to, selenocysteine, pyrrolysine, and N-formylmethionine, β-amino acids, Homo-amino acids, Proline and Pyruvic acid derivatives, 3-substituted alanine derivatives, glycine derivatives, ring-substituted phenylalanine and tyrosine derivatives, linear core amino acids, N-methyl amino acids. The term “amino acid residue” refers to an amino acid incorporated into a polypeptide that forms peptide bond(s) with neighboring amino acid(s).

As used herein, the term “post-translational modification” refers to modifications that occur on a peptide after its translation, e.g., translation by ribosomes, is complete. A post-translational modification may be a covalent chemical modification or enzymatic modification. Examples of post-translation modifications include, but are not limited to, acylation, acetylation, alkylation (including methylation), biotinylation, butyrylation, carbamylation, carbonylation, deamidation, deiminiation, diphthamide formation, disulfide bridge formation, eliminylation, flavin attachment, formylation, gamma-carboxylation, glutamylation, glycylation, glycosylation, glypiation, heme C attachment, hydroxylation, hypusine formation, iodination, isoprenylation, lipidation, lipoylation, malonylation, methylation, myristolylation, oxidation, palmitoylation, pegylation, phosphopantetheinylation, phosphorylation, prenylation, propionylation, retinylidene Schiff base formation, S-glutathionylation, S-nitrosylation, S-sulfenylation, selenation, succinylation, sulfination, ubiquitination, and C-terminal amidation. A post-translational modification includes modifications of the amino terminus and/or the carboxyl terminus of a peptide. Modifications of the terminal amino group include, but are not limited to, des-amino, N-lower alkyl, N-di-lower alkyl, and N-acyl modifications. Modifications of the terminal carboxy group include, but are not limited to, amide, lower alkyl amide, dialkyl amide, and lower alkyl ester modifications (e.g., wherein lower alkyl is C1-C4 alkyl). A post-translational modification also includes modifications, such as but not limited to those described above, of amino acids falling between the amino and carboxy termini. The term post-translational modification can also include peptide modifications that include one or more detectable labels.

The term “detectable label” as used herein refers to a substance which can indicate the presence of another substance when associated with it. The detectable label can be a substance that is linked to or incorporated into the substance to be detected. In some embodiments, a detectable label is suitable for allowing for detection and also quantification, for example, a detectable label that emitting a detectable and measurable signal. Examples of detectable labels include a dye, a fluorophore, a chromophore, a fluorescent nanoparticle (e.g. quantum dot), a radiolabel, an enzyme (e.g. alkaline phosphatase, luciferase or horseradish peroxidase), or a chemiluminescent or bioluminescent molecule.

As used herein, the term “linker” refers to one or more of a nucleotide, a nucleotide analog, an amino acid, a peptide, a polypeptide, a polymer, or a non-nucleotide chemical moiety that is used to join two molecules. A linker may be used to join a recording tag with a polypeptide, a polypeptide with a support, a recording tag with a solid support, etc. In certain embodiments, a linker joins two molecules via enzymatic reaction or chemistry reaction (e.g., a click chemistry reaction). In certain embodiments, the nucleic acid recording tag is associated directly or indirectly to the polypeptide analyte via a non-nucleotide chemical moiety.

The terminal amino acid at one end of a peptide or polypeptide chain that has a free amino group is referred to herein as the “N-terminal amino acid” (NTAA). The terminal amino acid at the other end of the chain that has a free carboxyl group is referred to herein as the “C-terminal amino acid” (CTAA). In certain embodiments, an NTAA, CTAA, or both may be modified or labeled with a moiety or a chemical moiety.

As used herein, the term “barcode” refers to a nucleic acid molecule of about 2 to about 30 bases (e.g., 2, 3, 4, 5, 6, 7, 8, 9, 10, 11, 12, 13, 14, 15, 16, 17, 18, 19, 20, 21, 22, 23, 24, 25, 26, 27, 28, 29 or 30 bases) providing a unique identifier tag or origin information for a polypeptide, a coding tag, a plurality of coding tags from an encoding cycle, a sample polypeptides, a set of samples, polypeptides within a compartment (e.g., droplet, bead, or separated location), polypeptides within a set of compartments, a fraction of polypeptides, a spatial region or set of spatial regions. A barcode can be an artificial sequence or a naturally occurring sequence. In certain embodiments, each barcode within a population of barcodes is different. In other embodiments, a portion of barcodes in a population of barcodes is different, e.g., at least about 10%, 15%, 20%, 25%, 30%, 35%, 40%, 45%, 50%, 55%, 60%, 65%, 70%, 75%, 80%, 85%, 90%, 95%, 97%, or 99% of the barcodes in a population of barcodes is different. A population of barcodes may be randomly generated or non-randomly generated. In certain embodiments, a population of barcodes are error-correcting or error-tolerant barcodes. Barcodes can be used to computationally deconvolute the multiplexed sequencing data and identify sequence reads derived from an individual polypeptide, sample, library, etc. A barcode can also be used for deconvolution of a collection of coding tags configured to react selectively with a specific type of amino acid residue(s) present in an immobilized polypeptide.

As used herein, the term “coding tag” refers to a polynucleotide with any suitable length, e.g., a nucleic acid molecule of about 2 bases to about 50 bases, including any integer including 2 and 50 and in between, that comprises a barcode region with identifying information regarding the specific type of amino acid residue(s) to which the coding tag reacts selectively. A “coding tag” may also be made from a “sequenceable polymer” (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety). A coding tag may comprise an encoder sequence, which is optionally flanked by one spacer on one side or optionally flanked by a spacer on each side. A coding tag may also be comprised of an optional UMI and/or an optional binding cycle-specific barcode. A coding tag is single stranded.

As used herein, the term “spacer” (Sp) refers to a nucleic acid molecule of about 1 base to about 20 bases (e.g., 1, 2, 3, 4, 5, 6, 7, 8, 9, 10, 11, 12, 13, 14, 15, 16, 17, 18, 19, or 20 bases) in length that is present on a terminus of a recording tag, or coding tag, or complementary coding tag. In certain embodiments, a spacer sequence flanks a barcode region of a coding tag on one end or both ends. Following removal of complementary coding tags that are not covalently coupled to the NTAA of the polypeptide, and providing conditions for nucleic acid hybridization between complementary coding tag attached to the NTAA and the recording tag, annealing between complementary spacer sequences on the recording tag and complementary coding tag allows transfer of identifying information through a primer extension reaction or ligation to the recording tag. Sp′ refers to spacer sequence complementary to Sp. Preferably, spacer sequences within a plurality of complementary coding tags possess the same number of bases. A common (shared or identical) spacer may be used in a plurality of complementary coding tags. A spacer sequence may have a “cycle specific” sequence in order to track complementary coding tags used in a particular encoding cycle. Only the sequential binding of correct cognate pairs results in interacting spacer elements and effective primer extension. A spacer sequence may comprise sufficient number of bases to anneal to a complementary spacer sequence in a recording tag to initiate a primer extension (also referred to as polymerase extension) reaction, or provide a “splint” for a ligation reaction.

As used herein, the term “recording tag” refers to a moiety, e.g., a chemical coupling moiety, a nucleic acid molecule, or a sequenceable polymer molecule (see, e.g., Niu et al., 2013, Nat. Chem. 5:282-292; Roy et al., 2015, Nat. Commun. 6:7237; Lutz, 2015, Macromolecules 48:4759-4767; each of which are incorporated by reference in its entirety) to which identifying information of a coding tag can be transferred, or from which identifying information about the polypeptide associated with the recording tag can be transferred to the coding tag. Identifying information can comprise any information characterizing a molecule such as information pertaining to sample, fraction, partition, spatial location, interacting neighboring molecule(s), cycle number, etc. Additionally, the presence of UMI can also be classified as identifying information. A recording tag may be directly linked to a polypeptide, linked to a polypeptide via a multifunctional linker, or associated with a polypeptide by virtue of its proximity (or co-localization) on a support. A recording tag may be linked via its 5′ end or 3′ end or at an internal site, as long as the linkage is compatible with the method used to transfer coding tag information to the recording tag or vice versa. A recording tag may further comprise other functional components, e.g., a universal priming site, unique molecular identifier, a barcode (e.g., a sample barcode, a fraction barcode, spatial barcode, a compartment tag, etc.), a spacer sequence that is complementary to a spacer sequence of a coding tag, or any combination thereof. The spacer sequence of a recording tag is preferably at the 3′-end of the recording tag in embodiments where polymerase extension is used to transfer coding tag information to the recording tag.

As used herein, the term “unique molecular identifier” or “UMI” refers to a nucleic acid molecule of about 3 to about 40 bases (3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases) in length providing a unique identifier tag for each polypeptide or complementary coding tagto which the UMI is linked. A polypeptide UMI can be used to computationally deconvolute sequencing data from a plurality of extended recording tags to identify extended recording tags that originated from an individual polypeptide. A polypeptide UMI can be used to accurately count originating polypeptide molecules by collapsing NGS reads to unique UMIs. A complementary coding tag UMI can be used to identify each individual complementary coding tag that is used during encoding.

As used herein, the term “universal priming site” or “universal primer” or “universal priming sequence” refers to a nucleic acid molecule, which may be used for library amplification and/or for sequencing reactions. A universal priming site may include, but is not limited to, a priming site (primer sequence) for PCR amplification, flow cell adaptor sequences that anneal to complementary oligonucleotides on flow cell surfaces enabling bridge amplification in some next generation sequencing platforms, a sequencing priming site, or a combination thereof. Universal priming sites can be used for other types of amplification, including those commonly used in conjunction with next generation digital sequencing. For example, extended recording tag molecules may be circularized and a universal priming site used for rolling circle amplification to form DNA nanoballs that can be used as sequencing templates (Drmanac et al., 2009, Science 327:78-81).

As used herein, the term “extended recording tag” refers to a recording tag to which information of at least one coding tag (a barcode region or its complementary sequence) has been transferred following binding of the complementary coding tag to the NTAA of the immobilized polypeptide. Information of the coding tag may be transferred to the recording tag directly (e.g., ligation) or indirectly (e.g., primer extension). Information of a coding tag may be transferred to the recording tag enzymatically or chemically. An extended recording tag may comprise complementary coding tag information of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 200 or more coding tags. The base sequence of an extended recording tag may reflect the sequential order of the specific amino acid residues identified by their coding tags configured to react selectively with them.

As used herein, the term “solid support”, “solid surface”, or “solid substrate”, or “sequencing substrate”, or “substrate” refers to any solid material, including porous and non-porous materials, to which a polypeptide can be associated directly or indirectly, by any means known in the art, including covalent and non-covalent interactions, or any combination thereof. A solid support may be two-dimensional (e.g., planar surface) or three-dimensional (e.g., gel matrix or bead). A solid support can be any support surface including, but not limited to, a bead, a microbead, an array, a glass surface, a silicon surface, a plastic surface, a filter, a membrane, a PTFE membrane, a silicon wafer chip, a flow through chip, a flow cell, a biochip including signal transducing electronics, a channel, a microtiter well, an ELISA plate, a spinning interferometry disc, a nitrocellulose-based polymer surface, a polymer matrix, a nanoparticle, or a microsphere. Materials for a solid support include but are not limited to acrylamide, agarose, cellulose, dextran, nitrocellulose, glass, gold, quartz, polystyrene, polyethylene vinyl acetate, polypropylene, polyester, polymethacrylate, polyacrylate, polyethylene, polyethylene oxide, polysilicates, polycarbonates, poly vinyl alcohol (PVA), Teflon, fluorocarbons, nylon, silicon rubber, polyanhydrides, polyglycolic acid, polyvinylchloride, polylactic acid, polyorthoesters, functionalized silane, polypropylfumerate, collagen, glycosaminoglycans, polyamino acids, dextran, or any combination thereof. Solid supports further include thin film, membrane, bottles, dishes, fibers, woven fibers, shaped polymers such as tubes, particles, beads, microspheres, microparticles, or any combination thereof. For example, when solid surface is a bead, the bead can include, but is not limited to, a ceramic bead, a polystyrene bead, a polymer bead, a polyacrylate bead, a methylstyrene bead, an agarose bead, a cellulose bead, a dextran bead, an acrylamide bead, a solid core bead, a porous bead, a paramagnetic bead, a glass bead, a controlled pore bead, a silica-based bead, or any combinations thereof. A bead may be spherical or an irregularly shaped. A bead or support may be porous. A bead's size may range from nanometers, e.g., 100 nm, to millimeters, e.g., 1 mm. In certain embodiments, beads range in size from about 0.2 micron to about 200 microns, or from about 0.5 micron to about 5 micron. In some embodiments, beads can be about 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter. In certain embodiments, “a bead” solid support may refer to an individual bead or a plurality of beads. In some embodiments, the solid surface is a nanoparticle. In certain embodiments, the nanoparticles range in size from about 1 nm to about 500 nm in diameter, for example, between about 1 nm and about 20 nm, between about 1 nm and about 50 nm, between about 1 nm and about 100 nm, between about 10 nm and about 50 nm, between about 10 nm and about 100 nm, between about 10 nm and about 200 nm, between about 50 nm and about 100 nm, between about 50 nm and about 150, between about 50 nm and about 200 nm, or between about 200 nm and about 500 nm in diameter. In some embodiments, the nanoparticles can be about 10 nm, about 50 nm, about 100 nm, about 150 nm, about 200 nm, about 300 nm, or about 500 nm in diameter. In some embodiments, the nanoparticles are less than about 200 nm in diameter.

As used herein, the term “nucleic acid molecule” or “polynucleotide” refers to a single- or double-stranded polynucleotide containing deoxyribonucleotides or ribonucleotides that are linked by 3′-5′ phosphodiester bonds, as well as polynucleotide analogs. A nucleic acid molecule includes, but is not limited to, DNA, RNA, and cDNA. A polynucleotide analog may possess a backbone other than a standard phosphodiester linkage found in natural polynucleotides and, optionally, a modified sugar moiety or moieties other than ribose or deoxyribose. Polynucleotide analogs contain bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide. Examples of polynucleotide analogs include, but are not limited to xeno nucleic acid (XNA), bridged nucleic acid (BNA), glycol nucleic acid (GNA), peptide nucleic acids (PNAs), morpholino polynucleotides, locked nucleic acids (LNAs), threose nucleic acid (TNA), 2′-O-Methyl polynucleotides, 2′-O-alkyl ribosyl substituted polynucleotides, phosphorothioate polynucleotides, and boronophosphate polynucleotides. A polynucleotide analog may possess purine or pyrimidine analogs, including for example, 7-deaza purine analogs, 8-halopurine analogs, 5-halopyrimidine analogs, or universal base analogs that can pair with any base, including hypoxanthine, nitroazoles, isocarbostyril analogues, azole carboxamides, and aromatic triazole analogues, or base analogs with additional functionality, such as a biotin moiety for affinity binding. In some embodiments, the nucleic acid molecule or oligonucleotide is a modified oligonucleotide. In some embodiments, the nucleic acid molecule or oligonucleotide is a DNA with pseudo-complementary bases, a DNA with protected bases, an RNA molecule, a BNA molecule, an XNA molecule, a LNA molecule, a PNA molecule, or a morpholino DNA, or a combination thereof. In some embodiments, the nucleic acid molecule or oligonucleotide is backbone modified, sugar modified, or nucleobase modified. In some embodiments, the nucleic acid molecule or oligonucleotide has nucleobase protecting groups such as Alloc, electrophilic protecting groups such as thiranes, acetyl protecting groups, nitrobenzyl protecting groups, sulfonate protecting groups, or traditional base-labile protecting groups.

As used herein, “nucleic acid sequencing” means the determination of the order of nucleotides in a nucleic acid molecule or a sample of nucleic acid molecules. Similarly, “polypeptide sequencing” means the determination of the identity and order of at least a portion of amino acids in the polypeptide molecule or in a sample of polypeptide molecules.

As used herein, “next generation sequencing” refers to high-throughput sequencing methods that allow the sequencing of millions to billions of molecules in parallel. Examples of next generation sequencing methods include sequencing by synthesis, sequencing by ligation, sequencing by hybridization, polony sequencing, ion semiconductor sequencing, and pyrosequencing. By attaching primers to a solid substrate and a complementary sequence to a nucleic acid molecule, a nucleic acid molecule can be hybridized to the solid substrate via the primer and then multiple copies can be generated in a discrete area on the solid substrate by using polymerase to amplify (these groupings are sometimes referred to as polymerase colonies or polonies). Consequently, during the sequencing process, a nucleotide at a particular position can be sequenced multiple times (e.g., hundreds or thousands of times) — this depth of coverage is referred to as “deep sequencing.” Examples of high throughput nucleic acid sequencing technology include platforms provided by Illumina, BGI, Qiagen, Thermo-Fisher, and Roche, including formats such as parallel bead arrays, sequencing by synthesis, sequencing by ligation, capillary electrophoresis, electronic microchips, “biochips,” microarrays, parallel microchips, and single-molecule arrays (See e.g., Service, Science (2006) 311:1544-1546).

As used herein, “analyzing” the polypeptide means to identify, detect, quantify, characterize, distinguish, or a combination thereof, all or a portion of the components of the polypeptide. For example, analyzing a peptide, polypeptide, or protein includes determining all or a portion of the amino acid sequence (contiguous or non-continuous) of the peptide. Analyzing a polypeptide also includes partial identification of a component of the polypeptide. For example, partial identification of amino acids in the polypeptide protein sequence can identify an amino acid in the protein as belonging to a subset of possible amino acids. Analysis typically begins with analysis of the n NTAA, and then proceeds to the next amino acid of the peptide (i.e., n-1, n-2, n-3, and so forth). This is accomplished by elimination of then NTAA, thereby converting the n-1 amino acid of the peptide to an N-terminal amino acid (referred to herein as the “n-1 NTAA”). Analyzing the peptide may also include determining the presence and frequency of post-translational modifications on the peptide, which may or may not include information regarding the sequential order of the post-translational modifications on the peptide. Analyzing the peptide may also include determining the presence and frequency of epitopes in the peptide, which may or may not include information regarding the sequential order or location of the epitopes within the peptide. Analyzing the peptide may include combining different types of analysis, for example obtaining epitope information, amino acid sequence information, post-translational modification information, or any combination thereof.

The term “sequence identity” is a measure of identity between polypeptides at the amino acid level, and a measure of identity between nucleic acids at nucleotide level. The polypeptide sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned. “Sequence identity” means the percentage of identical subunits at corresponding positions in two sequences when the two sequences are aligned to maximize subunit matching, i.e., taking into account gaps and insertions. For example, the BLAST algorithm (NCBI) calculates percent sequence identity and performs a statistical analysis of the similarity and identity between the two sequences. The software for performing BLAST analysis is publicly available through the National Center for Biotechnology Information (NCBI) website.

The term “unmodified” (also “wild-type” or “native”) as used herein is used in connection with biological materials such as nucleic acid molecules and proteins (e.g., cleavase), refers to those which are found in nature and not modified by human intervention.

The term “modified” or “engineered” (or “variant”, or “mutant”) as used in reference to nucleic acid molecules and protein molecules, e.g., an engineered DNA polymerase, implies that such molecules are created by human intervention and/or they are non-naturally occurring. The variant, mutant or engineered DNA polymerase is a polypeptide having an altered amino acid sequence, relative to an unmodified or wild-type protein, such as starting DNA polymerase, or a portion thereof. An engineered enzyme is a polypeptide which differs from a wild-type enzyme scaffold sequence, or a portion thereof, by one or more amino acid substitutions, deletions, additions, or combinations thereof. An engineered DNA polymerase generally exhibits at least 70%, 80%, 85%, 86%, 87%, 88%, 89%, 90%, 91%, 92%, 93%, 94%, 95%, 96%, 97%, 98%, 99% or more sequence identity to a corresponding wild-type starting DNA polymerase scaffold. Non-naturally occurring amino acids as well as naturally occurring amino acids are included within the scope of permissible substitutions or additions. A variant or engineered DNA polymerase denotes a composition and not necessarily a product produced by any given process. A variety of techniques including genetic selection, protein engineering, recombinant methods, chemical synthesis, or combinations thereof, may be employed.

The term “template” as used herein refers to a double-stranded or single-stranded nucleic acid molecule which is to be amplified, synthesized or sequenced. In the case of a double-stranded DNA molecule, denaturation of its strands to form a first and a second strand is performed before these molecules may be amplified, synthesized or sequenced. A primer, complementary to a portion of a template is hybridized under appropriate conditions and the polymerase of the invention may then synthesize a molecule complementary to said template or a portion thereof. Mismatch incorporation or strand slippage during the synthesis or extension of the newly synthesized molecule may result in one or a number of mismatched base pairs.

As used herein “amplification” refers to any in vitro method for increasing the number of copies of a nucleotide sequence with the use of a DNA polymerase. Nucleic acid amplification results in the incorporation of nucleotides into a DNA molecule or primer thereby forming a new DNA molecule complementary to a DNA template. The formed DNA molecule and its template can be used as templates to synthesize additional DNA molecules.

The terms “hybridization” and “hybridizing” refers to the pairing of two complementary single-stranded nucleic acid molecules (RNA and/or DNA) to give a double-stranded molecule. As used herein, two nucleic acid molecules may be hybridized, although the base pairing is not completely complementary. Accordingly, mismatched bases do not prevent hybridization of two nucleic acid molecules provided that appropriate conditions, well known in the art, are used. In the present invention, the term “hybridization” refers particularly to hybridization of an oligonucleotide to a template molecule.

As used herein, the term “primer extension”, also referred to as “polymerase extension”, refers to a reaction catalyzed by a nucleic acid polymerase (e.g., DNA polymerase) whereby a nucleic acid molecule (e.g., oligonucleotide primer, spacer sequence) that anneals to a complementary strand is extended by the polymerase, using the complementary strand as template.

The term “3′→5′ exonuclease activity” refers to an enzymatic activity associated with DNA polymerases and is involved in a DNA replication “editing” or correction mechanism during template extension.

A “DNA polymerase substantially reduced in 3′-to-5′ (or 3′-5′) exonuclease activity” is defined herein as a DNA polymerase having a 3′-5′ exonuclease specific activity which is less than about 1 unit/mg protein, or preferably about or less than 0.1 units/mg protein. A unit of activity of 3′-5′ exonuclease is defined as the amount of activity that solubilizes 10 nmoles of substrate ends in 60 min at 37° C., assayed as described in the “BRL 1989 Catalogue & Reference Guide”, page 5, with HhaI fragments of lambda DNA 3′-end labeled with [³H]dTTP by terminal deoxynucleotidyl transferase (TdT). Non-limiting examples of commercially available DNA polymerases that have substantially reduced 3′→5′ exonuclease activity include Taq, Klenow fragment DNA polymerase, Tne(exo-), Tma(exo-), Pfu (exo-) DNA polymerases, and mutants, variants and derivatives thereof. Alternatively, a DNA polymerase with substantially reduced 3′-to-5′ exonuclease activity can be obtained by introducing mutation(s) to a DNA polymerase having 3′→5′ exonuclease activity, and can be defined as a mutated DNA polymerase that has about or less than 10%, or preferably about or less than 1%, of the 3′-5′ exonuclease activity of the corresponding unmutated, wildtype enzyme. For example, wildtype T5 DNA polymerase (T5-DNAP) has a specific activity of about 10 units/mg protein while the specific mutant of the DNA polymerase has a specific activity of about 0.0001 units/mg protein, a 10⁵-fold reduction in specific activity compared to the unmodified enzyme (U.S. Pat. No. 5,270,179). Other examples of DNA polymerases having substantially reduced 3′-to-5′ exonuclease activity and methods for producing them, as well as thermostable DNA polymerases that can be used as starting scaffolds are disclosed in U.S. Pat. Nos. 5,541,099, 5,436,149, 5,614,365, 5,374,553, 5,047,342, 4,889,818 and 7,501,237.

It is understood that aspects and embodiments of the invention described herein include “consisting of” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Disclosure of Different Embodiments of the Invention

In one embodiment of the invention, provided herein is a method for identifying a polypeptide, the method comprising the steps of: (a) providing the polypeptide and an associated recording tag joined to a solid support; (b) contacting the polypeptide with a plurality of coding tags, wherein each coding tag of the plurality of coding tags is configured to react selectively with a specific type of amino acid residue(s) and comprises a barcode region with identifying information regarding the specific type of amino acid residue(s) to which the coding tag reacts selectively, thereby obtaining the polypeptide comprising coding tags attached to the specific amino acid residues; (c) contacting the polypeptide comprising coding tags attached to the specific amino acid residues with a plurality of complementary coding tags, wherein each complementary coding tag of the plurality of complementary coding tags comprises (i) a region complementary to the barcode region of a corresponding coding tag, (ii) a first spacer region complementary to a first complementary spacer region of the recording tag, and (iii) a moiety configured, when in a close proximity, to be covalently coupled to the NTAA of the polypeptide, or to a modified NTAA of the polypeptide; (d) providing conditions for covalently coupling the moiety of complementary coding tags to the NTAA of the polypeptide or the modified NTAA of the polypeptide; (e) removing complementary coding tags that are not covalently coupled to the NTAA of the polypeptide; (f) transferring identifying information of the barcode region or the region complementary to the barcode region from the complementary coding tag covalently coupled to the NTAA of the polypeptide to the recording tag, wherein transferring the identifying information comprises a primer extension or ligation; (g) removing the NTAA of the polypeptide, thereby exposing a new NTAA; (h) adding a second order complementary spacer region to the recording tag extended at step (f); (j) repeating steps (c)-(h) one or more times by replacing at step (c) the first spacer region of the complementary coding tags with a second or higher order spacer region complementary to the second or higher order complementary spacer region of the recording tag, and by replacing at step (h) the second complementary spacer region with a third or higher order complementary spacer region; and (k) analyzing the recording tag extended at step (i) by a nucleic acid sequencing method, and obtaining information regarding the specific amino acid residues of the polypeptide; thereby identifying the polypeptide.

Another embodiment provided herein is a method for identifying a polypeptide, the method comprising the steps of: (a) providing the polypeptide and an associated recording tag joined to a solid support; (b) contacting the polypeptide with a plurality of coding tags, wherein each coding tag of the plurality of coding tags is configured to react selectively with a specific type of amino acid residue(s) and comprises: i) a barcode region with identifying information regarding the specific type of amino acid residue(s) to which the coding tag reacts selectively, ii) an identification region unique for each specific type of amino acid residue(s) to which the coding tags react selectively, and iii) a recognition region for a site-specific restriction enzyme, located between the barcode region and the identification region, thereby obtaining the polypeptide comprising coding tags attached to the specific amino acid residues; (c) providing conditions for hybridization of the recording tag to one of the coding tags attached to the specific amino acid residue(s), thereby forming a double stranded region, and extending the recording tag (using the coding tag as a template), thereby transferring information of the barcode region and the recognition region from the coding tag to the recording tag; (d) cutting the recognition region by providing the site-specific restriction enzyme, so that the extended recording tag is released and only a coding tag stub comprising the identification region of the coding tag remains attached to the polypeptide; (e) repeating steps (c) and (d) for all other coding tags attached to the specific amino acid residues of the polypeptide, thereby obtaining the polypeptide comprising coding tag stubs attached to the specific amino acid residues; (f) removing the NTAA of the polypeptide, thereby exposing a new NTAA; (g) adding a second order complementary spacer region to the recording tag extended at step (f); (h) restoring coding tags from the coding tag stubs using the identification region; (j) repeating steps (c)-(h) one or more times; and (k) analyzing the recording tag extended at step (j) by a nucleic acid sequencing method, and obtaining information regarding the specific amino acid residues of the polypeptide before and after removing step, thereby identifying the polypeptide.

Various embodiments apply equally to the aspects provided herein but will for the sake of brevity be recited only once. Thus, various of the following embodiments apply equally to aspects recited below. It is also to be understood that, while methods are described in the context of a polypeptide, such methods are also directed to identifying a plurality of polypeptides. Thus, in many aspects, the methods comprise a first step of providing polypeptides, wherein each polypeptide is associated with a recording tag immobilized on a solid support, followed by a second step of contacting at least a subset of the polypeptides with a plurality of the coding tags.

In some embodiments, the disclosed methods are capable of identifying 100 or more, 1000 or more, or 10,000 or more different polypeptides simultaneously in a single assay (multiplexing).

In some embodiments, for identification of a polypeptide, the polypeptide is contacted with a plurality of coding tags, wherein each coding tag of the plurality of coding tags is configured to react selectively with a specific type of amino acid residues. Each coding tag is configured to selectively label a single type of amino acid residues, or a few types of amino acid residues. For example, some coding tags can be designed to selectively label both glutamate and aspartate residues. Either single or more than one amino acid residues of specific type can be present in a given immobilized peptide. Various methods and reagents known in the art can be used to design attachment of coding tags selectively to the specific amino acid residues of the polypeptide. For example, lysine specific reagents and disclosed in U.S. Pat. No. 7163803 B2 and published patent application US 20180372751 A1. Some of the known reagents that target cysteines contain maleimide and iodoacetamide reactive groups. In another example, the phenol ring of tyrosine can be labeled using benzyl diazo groups (Gavrilyuk J., et al., Formylbenzene diazonium hexafluorophosphate reagent for tyrosine-selective modification of proteins and the introduction of a bioorthogonal aldehyde. Bioconjugate Chem. 2012, 23, 2321-2328). In another example, tyrosine side chains can be selectively targeted with cyclic diazodicaboxamides in aqueous buffer (Ban H., et al., Tyrosine bioconjugation through aqueous ene-type reactions: a click-like reaction for tyrosine. J. Am. Chem. Soc. 2010, 132, 1523-1525). In still another example, histidine can be selectively labeled with thiophosphorodichloridates (Jia S., et al., Bioinspired thiophosphorodichloridate reagents for chemoselective histidine bioconjugation. J. Am. Chem. Soc. 2019, 141, 7294-7301).

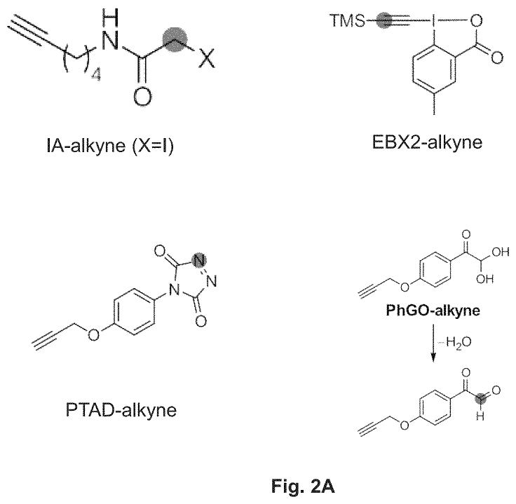

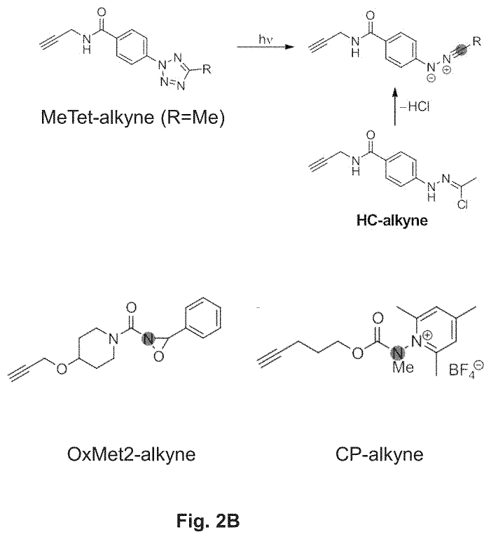

Recently, reactive alkyne probes have been developed to specifically target particular amino acid types in proteins and were evaluated on broad sets of protein targets (Zanon PRA, et al. Profiling the proteome-wide selectivity of diverse electrophiles. ChemRxiv. Cambridge: Cambridge Open Engage; 2021; Gehringer, M. & Laufer, S. A. Emerging and Re-Emerging Warheads for Targeted Covalent Inhibitors: Applications in Medicinal Chemistry and Chemical Biology. J. Med. Chem. 2019, 62, 5673-5724; Parker, C. G. & Pratt, M. R. Click Chemistry in Proteomic Investigations. Cell 2020, 180, 605-632). Some examples of such reactive alkyne probes include: IA-alkyne and EBX2-alkyne for cysteines; STP-alkyne, ArSq-alkyne and EBA-alkyne for lysines; PTAD-alkyne and SuTEx2-alkyne for tyrosines; HC-alkyne, MeTet-alkyne and Az-alkyne for aspartates and glutamates; OxMet2-alkyne for methionines; CP-alkyne, HMN-alkyne, MMP-alkyne for tryptophans; and CP-alkyne for histidines; PhGO-alkyne for arginines (see FIG. 2A-2B and Zanon PRA, et al. Profiling the proteome-wide selectivity of diverse electrophiles. ChemRxiv. Cambridge: Cambridge Open Engage; 2021). Thus, reactive alkyne probes can be used to react with polypeptides in a residue-specific fashion and can be utilized to selectively attach nucleic acid coding tags to eight specific types of amino acid residues listed above.

In some embodiments, the specific amino acid residues are amine-containing amino acid residues, such as lysines. In some embodiments, coding tags of the plurality of coding tags are attached to the amine-containing amino acid residues through an NHS-ester or an imidoester.

In some embodiments, the specific amino acid residues are sulfhydryl-containing amino acid residues, such as cysteines. In some embodiments, coding tags of the plurality of coding tags are attached to the sulfhydryl-containing amino acid residues through a maleimide group, a haloacetyl group, or a pyridyldisulfide.

In some embodiments, the specific amino acid residues are carboxyl-containing amino acid residues, such as aspartate or glutamate. In some embodiments, coding tags of the plurality of coding tags are attached to the carboxyl-containing amino acid residues through a carbodiimide.

In some embodiments, the specific type of amino acid residues to which coding tags can be selectively attached is selected from the group consisting of: lysine, arginine, aspartate, glutamate, histidine, cysteine, serine, methionine, tryptophan and tyrosine.

In some embodiments, a specific type of amino acid residues is modified prior to attachment of nucleic acid coding tags. For example, a specific type of amino acid residues can be modified with a reactive handle, and then the reactive handle can react with modified nucleic acid coding tags covalently attaching them to the specific type of amino acid residues. Some exemplary reactive handle useful for chemical coupling between polynucleotides and amino acid residues are disclosed in US 10006917 B2, US 10697974 B2 and in US provisional application #63/225,274, incorporated herein.

In some embodiments, the polypeptide is modified with a modifying agent optionally comprising a click chemistry reactive group prior to or at the same time as nucleic acid coding tags are attached. Examples of click chemistry reactive groups that can be used include, without limitation, azide, semicarbazide, methyltetrazine, pyridyltetrazine, trans-cyclooctene (TCO), aTCO (functionalized axial-5-hydroxy-trans-cyclooctene), bicyclononyne (BCN), diarylcyclooctyne (DBCO), and/or alkyne. In some embodiments, the nucleic acid coding tags comprise a complementary click chemistry reactive group that will react with the click chemistry reactive group present in the modifier agent. In some embodiments, the counter-acting click chemistry groups are 1,2,4,5-tetrazine.

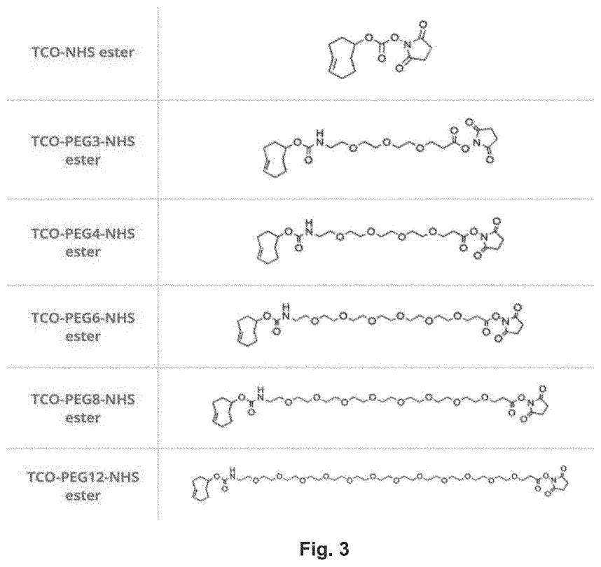

In some embodiments, specific amino acid residues are attached to nucleic acid coding tags through a linker. In some embodiments, the linker is a PEG linker or an aliphatic linker. In some embodiments, the amino acid residue is lysine and is modified by any one of the agents shown in FIG. 3, wherein TCO is used to attach nucleic acid coding tags.

In some embodiments, the polypeptide is contacted with nucleic acid coding tags comprising a reactive handle, wherein the reactive handle is configured to be attached to a specific amino acid residue of the polypeptide, thereby obtaining the polypeptide comprising coding tags attached to the specific amino acid residues. In other embodiments of the method, specific amino acid residues of the polypeptide are contacted with a first reactive handle, wherein the first reactive handle is configured to be attached to the specific amino acid residues, thereby forming modified specific amino acid residues of the polypeptide; then the modified specific amino acid residues are contacted with nucleic acid coding tags comprising a second reactive handle, which is configured to specifically react with and/or specifically bind to the first reactive handle, thereby obtaining the polypeptide comprising coding tags attached to the specific amino acid residues. In some embodiments, the second reactive handle is attached to the first reactive handle via a linking moiety. In some embodiments, reaction between the first reactive handle and the second reactive handle is a bioorthogonal reaction.

In some cases, the first and/or second reactive handles used to attach specific amino acid residues of the polypeptide and coding tags comprise a bio-orthogonal reactive group (click chemistry reagent). In preferred embodiments, the bio-orthogonal reactive group is a reaction partner for an inverse electron demand Diels Alder (IEDDA) reaction. Some examples of bioorthogonal reactions that can be utilized herein are disclosed, for example, in U.S. Pat. No. 8,236,949 B2, U.S. Pat. No. 9,169,283 B2, U.S. Pat. No. 10,611,738 B2, U.S. Pat. No. 10,442,789 B2, and in Fox J M, et al., “General, Divergent Platform for Diastereoselective Synthesis of trans-Cyclooctenes with High Reactivity and Favorable Physiochemical Properties. Angew Chem Int Ed Engl. 2021 Mar. 19”.

In some particular embodiments, the specific amino acid residues are lysines; the first reactive handle is selected from the group consisting of: azide, semicarbazide, azidoacetamide, methyltetrazine, TCO, aTCO, and BCN; and the first reactive handle is attached the modified amino acid residue optionally through the linker comprising: amide, carbamate, thiourea, urea, amidine, guanidine, sulfonamide, sulfonimidamide, imine, alkylation, arylation, iminoboronate, or cyclic amide, wherein TCO is trans-cyclooctene, and aTCO is a functionalized axial-5-hydroxy-trans-cyclooctene (Fox J M, et al., “General, Divergent Platform for Diastereoselective Synthesis of trans-Cyclooctenes with High Reactivity and Favorable Physiochemical Properties. Angew Chem Int Ed Engl. 2021 Mar. 19”).

In some particular embodiments, the specific amino acid residues are arginines; the first reactive handle is selected from the group consisting of: azide, semicarbazide, azidoacetamide, methyltetrazine, TCO, aTCO, and BCN; and the first reactive handle is attached the modified amino acid residue optionally through the linker comprising cyclohexanediketone or aryl glyoxal.

In some particular embodiments, the specific amino acid residues are tyrosines; the first reactive handle is selected from the group consisting of: azide, semicarbazide, azidoacetamide, methyltetrazine, TCO, aTCO, and BCN; and the first reactive handle is attached the modified amino acid residue optionally through the linker comprising an aryl diazo or triazolinedione moiety.

In some particular embodiments, the specific amino acid residues are serines; the first reactive handle is selected from the group consisting of: azide, semicarbazide, azidoacetamide, methyltetrazine, TCO, aTCO, and BCN; and the first reactive handle is attached to the modified amino acid residue optionally through the linker comprising a phosphorus (V)-based (Psi) moiety.

In some particular embodiments, the specific amino acid residues are histidines; the first reactive handle is selected from the group consisting of: azide, semicarbazide, azidoacetamide, methyltetrazine, TCO, aTCO, and BCN; and the first reactive handle is attached to the nascent modified amino acid residue optionally through a linker comprising a thiophosphorochloridate moiety.

In some particular embodiments, the specific amino acid residues are cysteines; the first reactive handle is selected from the group consisting of: azide, semicarbazide, azidoacetamide, methyltetrazine, TCO, aTCO, and BCN; and the first reactive handle is attached to the nascent modified amino acid residue optionally through the linker comprising a maleimide or carbonylacrylic moiety.

In some particular embodiments, the specific amino acid residues are aspartates or glutamates; the first reactive handle is selected from the group consisting of: azide, semicarbazide, azidoacetamide, methyltetrazine, TCO, aTCO, and BCN; and the first reactive handle is attached to the modified amino acid residue optionally through a linker comprising an amide moiety or 2H-azirine-containing moiety. For labeling Glu or Asp, two exemplary methods can be employed. The first method is a two-step reaction: preactivating the carboxylic acids with a carbodiimide, then adding in an amine-bearing handle to form an amide moiety. The carboxyl may be activated as a carbodiimide intermediate or via activation with Woodward's reagent K (N-ethyl-5-phenylisoxazolium 3′-sulfonate). The other method is a one-step procedure that utilizes the 2H-azirine group-bearing functional handle.

In some particular embodiments, the specific amino acid residues are post-translational modified residues, such as phosphoserine or phosphothreonine; the first reactive handle is selected from the group consisting of: azide, semicarbazide, azidoacetamide, methyltetrazine, TCO, aTCO, and BCN; and the first reactive handle is attached to the nascent modified amino acid residue optionally through the linker comprising a diazo moiety.

In some embodiments, labeled specific amino acid residue of the polypeptide are obtained by modifying the polypeptide with a modifying agent. In some embodiments, the modification occurs as disclosed in exemplary chemical schemes below for particular amino acid residues. Examples of modifying agents are also shown. In the following structures,

PP represents a portion of the treated polypeptide;

LG represents a leaving group and is independently selected at each occurrence from a halogen (Cl, Br, etc.), triflate, tosylate, mesylate, N-hydroxysuccinimdyl, sulfo-N-hydroxysuccinimdyl, halogenated phenol, halogenated thiophenol, p-nitrophenol, hexafluoroisopropanol, or acetate;

PG represents a protecting group and can be independently selected at each occurrence from t-butyl carbamate (Boc), 9-fluorenylmethyl carbamate (Fmoc), 2,2,2-trichloroethoxycarbamate (Troc), 2-(trimethylsilyl)ethoxycarbamate (Teoc), 2,2,6,6-tetramethylpiperidin-1-yloxycarbamate (Tempoc), benzyl carbamate (Cbz), azidomethyl carbamate (Azoc), allyloxycarbamate (Alloc), or O-nitrobenzyloxy carbamate (oNZ);

X can be comprised independently of O or S;

R represents an optional spacer comprised of polyethylene glycol, polypropylene glycol, poly glycine, or longer chain alkyl chain attached to a bioorthogonal group optionally comprised of azide, semicarbazide, methyltetrazine, pyridyltetrazine, TCO, aTCO, BCN, DBCO, or alkyne. Exemplary amino acid-specific (or modified amino acid-specific) modification methods are shown below (specific amino acid residues are indicated):

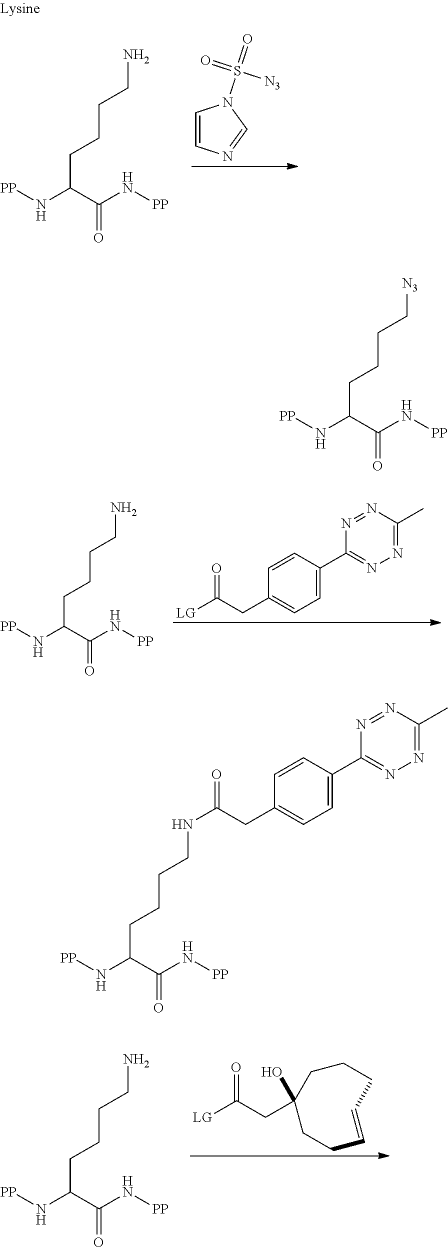

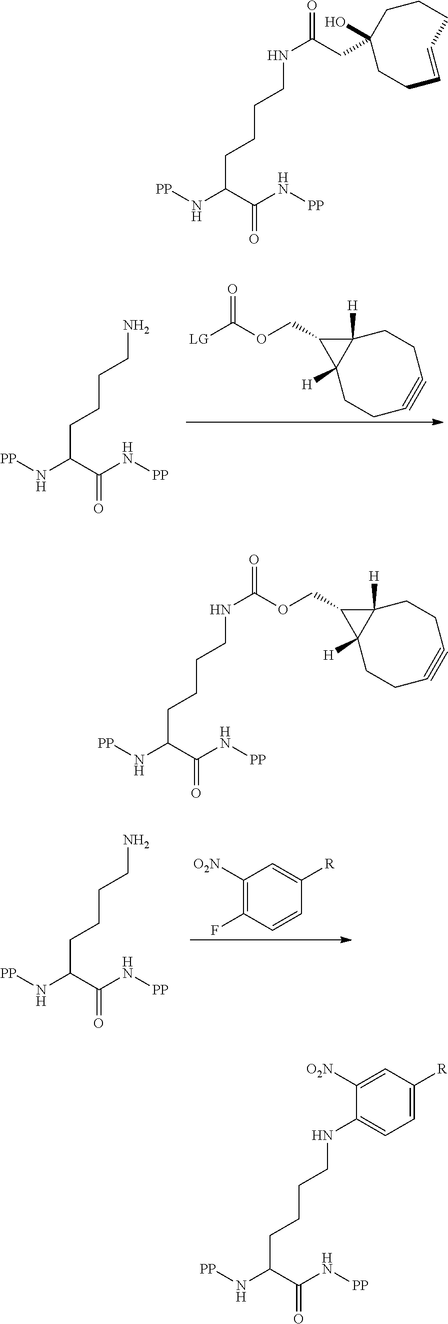

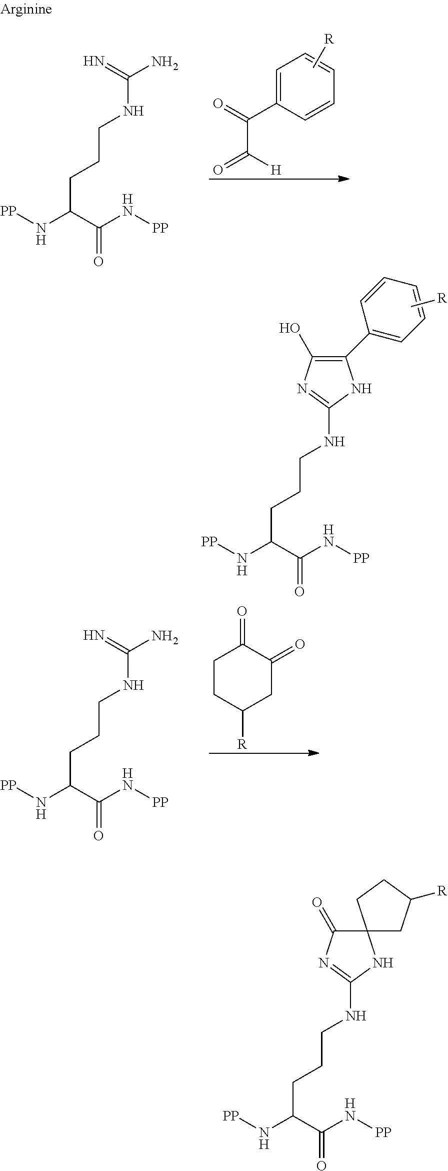

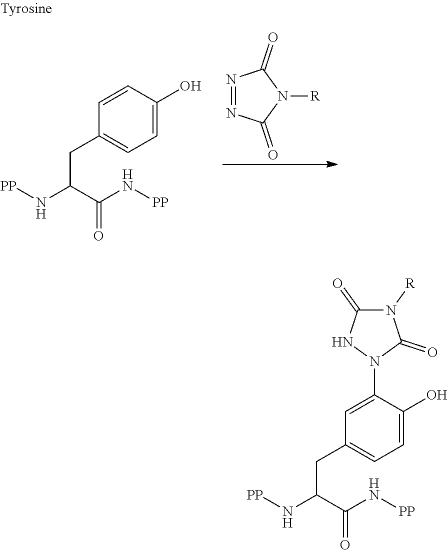

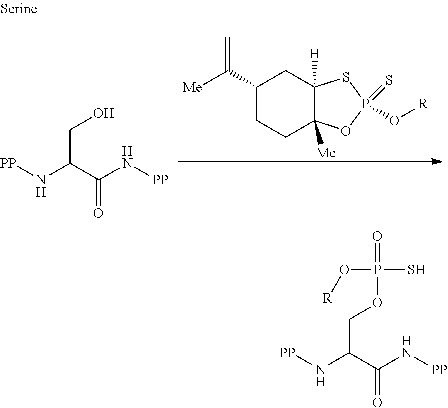

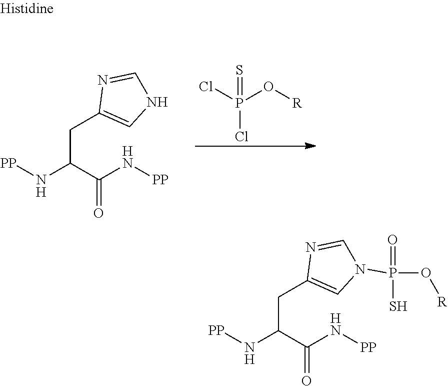

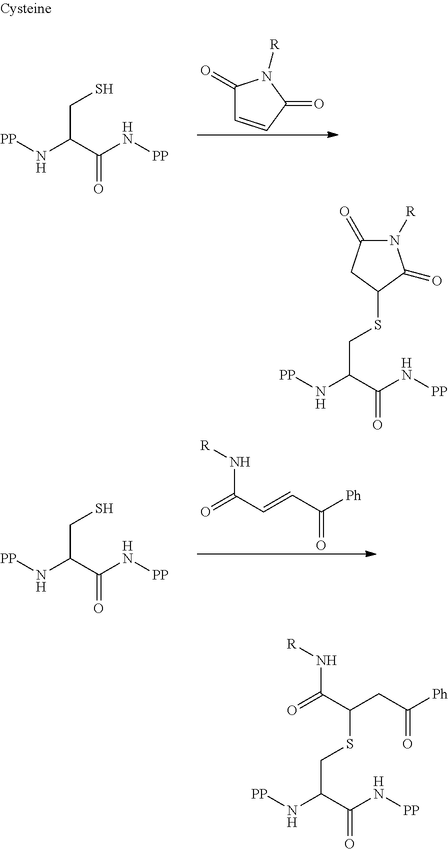

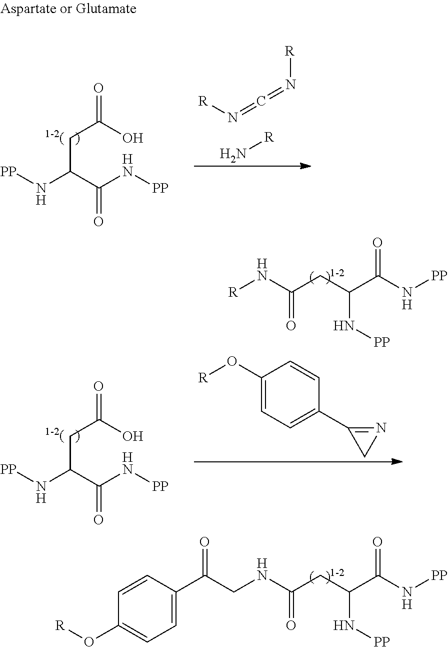

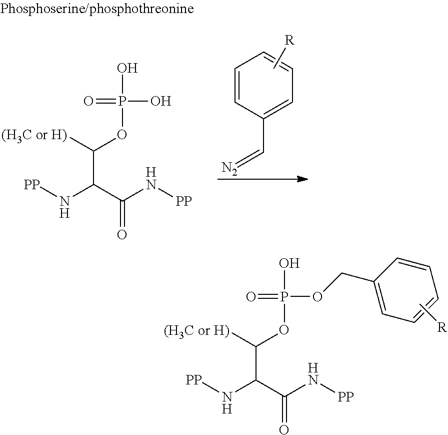

Amino acid residues buried within the protein core may not be accessible to coding tags. In one embodiment of the present invention, this may be resolved by denaturing large proteins or cleaving large proteins or large peptides into smaller peptides before proceeding with the reaction with the coding tags.

In some embodiments, the disclosed methods further include denaturing or partial unfolding the polypeptide to provide conditions for efficient labeling of the specific amino acid residues with the coding tags. Denaturing or partial unfolding the polypeptide can be achieved by exposure to a denaturing agent, to partly unfold the conformational structure of the native polypeptide. As defined herein, “partial unfolding” refers to a change in the conformation of a polypeptide so as to perturb the shape or conformation of the polypeptide without causing an irreversible denaturation of the polypeptide. “Conformation” refers to a combination of secondary and tertiary structure of a polypeptide. Alternatively, irreversible denaturation of the polypeptide can be used to provide access for the coding tags to specific, solvent-inaccessible amino acid residues of the polypeptide that are buried inside the structure under native conformation of the polypeptide. Examples of denaturing agents capable of denaturing or partial unfolding the polypeptide include, but not limited to inorganic salts, water-miscible organic solvents, acids, bases, surfactants, and chaotropic agents. Exemplary denaturing agents are: ethanol, dimethylsulfoxide, acetonitrile, urea, guanidine hydrochloride, sodium dodecyl sulfate, sodium lauroyl sarcosinate. Additional examples of denaturing agents are listed in U.S. Pat. No. 4,714,677 A and U.S. Pat. No.10,006,917 B2, incorporated herein. Alternatively, partial unfolding of a polypeptide can be accomplished by incubating the polypeptide in an aqueous solution at elevated temperatures, for example, in the range of about 25° C. to about 60° C.

In some embodiments, providing the polypeptide and an associated recording tag joined to a solid support comprises the following steps: attaching the polypeptide to the recording tag to generate a nucleic acid-polypeptide conjugate; bringing the nucleic acid-polypeptide conjugate into proximity with a solid support by hybridizing the recording tag in the nucleic acid-polypeptide conjugate to a capture nucleic acid attached to the solid support; and covalently coupling the nucleic acid-polypeptide conjugate to the solid support.

Recording tags can be attached to the polypeptide pre- or post-immobilization to the solid support. For example, polypeptides can be first labeled with recording tags and then immobilized to a solid surface via a recording tag comprising two functional moieties for coupling. One functional moiety of the recording tag couples to the polypeptide, and the other functional moiety immobilizes the recording tag-labeled polypeptide to a solid support. Alternatively, polypeptides are immobilized to a solid support prior to labeling with recording tags. For example, polypeptides can first be derivatized with reactive groups such as click chemistry moieties. The activated polypeptides molecules can then be attached to a suitable solid support and then labeled with recording tags using the complementary click chemistry moiety. As an example, polypeptides derivatized with alkyne and mTet moieties may be immobilized to beads derivatized with azide and TCO and attached to recording tags labeled with azide and TCO. It is understood that the methods provided herein for attaching polypeptides to the solid support may also be used to attach recording tags to the solid support or attach recording tags to polypeptides.

A recording tag can be joined to the solid support, directly or indirectly (e.g., via a linker), by any means known in the art, including covalent and non-covalent interactions, or any combination thereof. For example, the recording tag may be joined to the solid support by a ligation reaction. Alternatively, the solid support can include an agent or coating to facilitate joining, either direct or indirectly, of the recording tag, to the solid support. Strategies for immobilizing nucleic acid molecules to solid supports (e.g., beads) have been described in U.S. Pat. No. 5,900,481; Steinberg et al. (2004, Biopolymers 73:597-605), incorporated herein by reference in its entirety.

In some embodiments, the polypeptide is attached to the 3′ end of the recording tag. In other embodiments, the polypeptide is attached to the 5′ end of the recording tag. In yet other embodiments, the polypeptide is attached to an internal position of the recording tag.

In some embodiments, a barcode is attached to the nucleic acid-polypeptide conjugate, wherein the barcode comprises a compartment barcode, a partition barcode, a sample barcode, a fraction barcode, or any combination thereof.

In some embodiments, the recording tag is covalently attached to the polypeptide to generate the nucleic acid-polypeptide conjugate. In some embodiments, the recording tag and/or capture nucleic acid further comprises a universal priming site, wherein the universal priming site comprises a priming site for amplification, sequencing, or both.

In some embodiments, the capture nucleic acid is derivatized or comprises a moiety (e.g., a reactive coupling moiety) to allow binding to a solid support. In some embodiments, the capture nucleic acid comprises a moiety (e.g., a reactive coupling moiety) to allow binding to the recording tag. In some other embodiments, the recording tag is derivatized or comprises a moiety (e.g., a reactive coupling moiety) to allow binding to a solid support. Methods of derivatizing a nucleic acid for binding to a solid support and reagents for accomplishing the same are known in the art. For this purpose, any reaction which is preferably rapid and substantially irreversible can be used to attach nucleic acids to the solid support. The capture nucleic acid may be bound to a solid support through covalent or non-covalent bonds. In a preferred embodiment, the capture nucleic acid is covalently bound to biotin to form a biotinylated conjugate. The biotinylated conjugate is then bound to a solid surface, for example, by binding to a solid, insoluble support derivatized with avidin or streptavidin. The capture nucleic acid can be derivatized for binding to a solid support by incorporating modified nucleic acids in the loop region. In other embodiments, the capture moiety is derivatized in a region other than the loop region.

Exemplary bioorthogonal reactions that can be used for binding to a solid support or for generating nucleic acid-polypeptide conjugates include the copper catalyzed reaction of an azide and alkyne to form a triazole (Huisgen 1, 3-dipolar cycloaddition), strain-promoted azide alkyne cycloaddition (SPAAC), reaction of a diene and dienophile (Diels-Alder), strain-promoted alkyne-nitrone cycloaddition, reaction of a strained alkene with an azide, tetrazine or tetrazole, alkene and azide [3+2] cycloaddition, alkene and tetrazine inverse electron demand Diels-Alder (IEDDA) reaction (e.g., m-tetrazine (mTet) or phenyl tetrazine (pTet) and trans-cyclooctene (TCO); or pTet and an alkene), alkene and tetrazole photoreaction, Staudinger ligation of azides and phosphines, and various displacement reactions, such as displacement of a leaving group by nucleophilic attack on an electrophilic atom (Horisawa 2014, Knall, Hollauf et al. 2014). Exemplary displacement reactions include reaction of an amine with: an activated ester; an N-hydroxysuccinimide ester; an isocyanate; an isothioscyanate, an aldehyde, an epoxide, or the like. In some embodiments, iEDDA click chemistry is used for immobilizing polypeptides to a solid support or for generating nucleic acid-polypeptide conjugates since it is rapid and delivers high yields at low input concentrations. In another embodiment, m-tetrazine rather than tetrazine is used in an iEDDA click chemistry reaction, as m-tetrazine has improved bond stability. In another embodiment, phenyl tetrazine (pTet) is used in an iEDDA click chemistry reaction.

In some embodiments, a plurality of capture nucleic acids are coupled to the solid support. In some cases, the sequence region that is complementary to the recording tag on the capture nucleic acids is the same among the plurality of capture nucleic acids. In some cases, the recording tag attached to various polypeptides comprises the same complementary sequence to the capture nucleic acid.

In some embodiments, the surface of the solid support is passivated (blocked). A “passivated” surface refers to a surface that has been treated with outer layer of material. Methods of passivating surfaces include standard methods from the fluorescent single molecule analysis literature, including passivating surfaces with polymer like polyethylene glycol (PEG) (Pan et al., 2015, Phys. Biol. 12:045006), polysiloxane (e.g., Pluronic F-127), star polymers (e.g., star PEG) (Groll et al., 2010, Methods Enzymol. 472:1-18), hydrophobic dichlorodimethylsilane (DDS)+self-assembled Tween-20 (Hua et al., 2014, Nat. Methods 11:1233-1236), diamond-like carbon (DLC), DLC+PEG (Stavis et al., 2011, Proc. Natl. Acad. Sci. USA 108:983-988), and zwitterionic moieties (e.g., U.S. Patent Application Publication US 2006/0183863). In addition to covalent surface modifications, a number of passivating agents can be employed as well including surfactants like Tween-20, polysiloxane in solution (Pluronic series), poly vinyl alcohol (PVA), and proteins like BSA and casein. Alternatively, density of polypeptides (e.g., proteins, polypeptide, or peptides) can be titrated on the surface or within the volume of a solid substrate by spiking a competitor or “dummy” reactive molecule when immobilizing the proteins, polypeptides or peptides to the solid substrate. In some embodiments, PEGs of various molecular weights can also be used for passivation from molecular weights of about 300 Da to 50 kDa or more.

In certain embodiments where multiple nucleic acid-polypeptide conjugates are immobilized on the same solid support, the nucleic acid-polypeptide conjugates can be spaced appropriately to accommodate methods of identification to be used. For example, it may be advantageous to space the nucleic acid-polypeptide conjugates that optimally to allow a nucleic acid-based method for identifying the polypeptides to be performed. In some embodiments, the method for identifying the polypeptides involve transferring information of the barcode region or the region complementary to the barcode region from complementary coding tag covalently coupled to the NTAA of the polypeptide to the recording tag, and information transfer from the complementary coding tag may reach a neighboring recording tag.

In certain embodiments where a plurality of the nucleic acid-polypeptide conjugates are immobilized on the same solid support, the conjugates or corresponding polypeptides can be spaced appropriately to reduce the occurrence of or prevent a cross-binding or inter-molecular event, e.g., where a complementary coding tag is attached to a first polypeptide and its barcode region information is transferred to a recording tag associated with a neighboring polypeptide rather than the recording tag associated with the first polypeptide. To control polypeptide spacing on the solid support, the density of functional coupling groups (e.g., TCO or capture DNA molecules) may be titrated on the substrate surface. In some embodiments, multiple polypeptides or adjacent polypeptides are spaced apart on the surface or within the volume (e.g., porous supports) of a solid support at a distance of about 50 nm to about 500 nm, or about 50 nm to about 400 nm, or about 50 nm to about 300 nm, or about 50 nm to about 200 nm, or about 50 nm to about 100 nm. In some embodiments, multiple polypeptides or adjacent polypeptides are spaced apart on the surface or within the volume of a solid support with an average distance of at least 50 nm, at least 60 nm, at least 70 nm, at least 80 nm, at least 90 nm, at least 100 nm, at least 150 nm, at least 200 nm, at least 250 nm, at least 300 nm, at least 350 nm, at least 400 nm, at least 450 nm, or at least 500 nm. In some embodiments, multiple polypeptides or adjacent polypeptides are spaced apart on the surface or within the volume of a solid support with an average distance of at least 50 nm. In some embodiments, polypeptides or adjacent polypeptides are spaced apart on the surface or within the volume of a solid support such that, empirically, the relative frequency of inter- to intra-molecular events is <1:10; <1:100; <1:1,000; or <1:10,000. A suitable spacing frequency can be determined empirically using a functional assay (as described, for example, in the published patent application US 20190145982 A1), and can be accomplished by dilution and/or by spiking a “dummy” spacer molecule that competes for attachments sites on the substrate surface. In some embodiments, when a plurality of the nucleic acid-polypeptide conjugates is coupled, any nucleic acid-polypeptide conjugates adjacently coupled on the solid support are spaced apart from each other at an average distance of about 50 nm or greater.

In some embodiments, the spacing of the polypeptide on the solid support is achieved by controlling the concentration and/or number of capture nucleic acids on the solid support. In some embodiments, any adjacently coupled capture nucleic acids are spaced apart from each other on the surface or within the volume (e.g., porous supports) of a solid support at a distance of about 50 nm, about 100 nm, or about 200 nm. In some embodiments, any adjacently coupled capture nucleic acids are spaced apart from each other on the surface of a solid support with an average distance of at least 50 nm. In some embodiments, any adjacently coupled capture nucleic acids are spaced apart from each other on the surface or within the volume of a solid support such that, empirically, the relative frequency of inter- to intra-molecular events (e.g. transfer of information) is <1:10; <1:100; <1:1,000; or <1:10,000.

A suitable spacing frequency can be determined empirically using a functional assay and can be accomplished by dilution and/or by spiking a “dummy” spacer molecule that competes for attachments sites on the substrate surface. For example, PEG-5000 (MW˜5000) is used to block the interstitial space between peptides on the substrate surface (e.g., bead surface). In addition, the peptide is coupled to a functional moiety that is also attached to a PEG-5000 molecule. In some embodiments, the functional moiety is an aldehyde, an azide/alkyne, or a malemide/thiol, or an epoxide/nucleophile, or an inverse electron demand Diels-Alder (iEDDA) group, or a moiety for a Staudinger reaction. In some embodiments, the functional moiety is an aldehyde group. In a preferred embodiment, this is accomplished by coupling a mixture of NHS-PEG-5000-TCO+NHS-PEG-5000-Methyl to amine-derivatized beads. The stoichiometric ratio between the two PEGs (TCO vs. methyl) is titrated to generate an appropriate density of functional coupling moieties (TCO groups) on the substrate surface; the methyl-PEG is inert to coupling. The effective spacing between TCO groups can be calculated by measuring the density of TCO groups on the surface. In certain embodiments, the mean spacing between coupling moieties (e.g., TCO) on the solid surface is at least 50 nm, at least 100 nm, at least 250 nm, or at least 500 nm. After PEG5000-TCO/methyl derivatization of the beads, the excess NH₂ groups on the surface are quenched with a reactive anhydride (e.g. acetic or succinic anhydride).

In some embodiments, the spacing is accomplished by titrating the ratio of available attachment molecules on the substrate surface. In some examples, the substrate surface (e.g., bead surface) is functionalized with a carboxyl group (COOH) which is treated with an activating agent (e.g., activating agent is EDC and Sulfo-NHS). In some examples, the substrate surface (e.g., bead surface) comprises NHS moieties. In some embodiments, a mixture of mPEGₙ-NH₂ and NH₂-PEGₙ-mTet is added to the activated beads (wherein n is any number, e.g., any number from n=1 to n=100 or more). In one example, the ratio between the mPEG₃-NH₂ (not available for coupling) and NH₂-PEG₄-mTet (available for coupling) is titrated to generate an appropriate density of functional moieties available to attach the polypeptide on the substrate surface. In certain embodiments, the mean spacing between coupling moieties (e.g., NH₂-PEG₄-mTet) on the solid surface is at least 50 nm, at least 100 nm, at least 250 nm, or at least 500 nm. In some specific embodiments, the ratio of NH₂-PEGₙ-mTet to mPEGₙ-NH₂ is about or greater than 1:1000, about or greater than 1:10,000, about or greater than 1:100,000, or about or greater than 1:1,000,000. In some further embodiments, the capture nucleic acid attaches to the NH₂-PEGₙ-mTet.

In some embodiments, addition of complementary spacer regions to the recording tag extended at step (f) at each cycle is performed by splint ligation. The splint ligation adds a hybridizing spacer sequence to the recording tag (RT) complementary to a corresponding spacer sequence present in complementary coding tag, and the hybridizing spacer sequence will be used for transferring barcode information to the RT on the encoding cycle(s).

In some embodiments, each complementary coding tag of the plurality of complementary coding tags comprises (i) a region complementary to the barcode region of a corresponding coding tag, (ii) a first spacer region complementary to a first complementary spacer region of the recording tag, and (iii) a moiety configured, when in a close proximity, to be covalently coupled to the NTAA of the polypeptide. In one embodiment, moiety is configured, when in a close proximity, to be covalently coupled to the amino group of the NTAA of the polypeptide or to a modified NTAA of the polypeptide. In one preferred embodiment, the polypeptide comprising coding tags attached to the specific amino acid residues is contacted with a NTAA modifier, which modifies the NTAA with a reactive group configured to react, when in a close proximity, with the moiety of the complementary coding tags. Only moiety located on the complementary coding tag attached to the NTAA can react with the reactive group of the NTAA modifier. In preferred embodiments, the moiety can react with the reactive group of the NTAA modifier when located at a distance of no more than 10 nm, more preferably no more than 5 nm. Therefore, moieties located on complementary coding tags attached to amino acid residues of the polypeptide other than the NTAA residue cannot react with the reactive group of the NTAA modifier.

In some embodiments, the moiety configured, when in a close proximity, to be covalently coupled to the NTAA of the polypeptide, or to a modified NTAA of the polypeptide, comprises a click chemistry reactive group. In some particular embodiments, the moiety comprises an azide and is configured to be covalently coupled to the NTAA of the polypeptide modified with a propargyl halide or an activated propargylic acid. In other particular embodiments, the moiety comprises a propargyl halide or an activated propargylic acid and is configured to be covalently coupled to the NTAA of the polypeptide modified with an azide.

In some embodiments, the NTAA of the polypeptide is modified with a modifying agent, which generates a modified NTAA. The complementary coding tags comprise a moiety configured, when in a close proximity, to be covalently coupled to the modified NTAA of the immobilized polypeptide. The non-limiting examples of the modifying agent are propargyl halides and 1-formyl-1-butyne with sodium cyanoborohydride, and the moiety comprises an azide group. These biorthogonal groups could also be reversed. Exemplary coupling reactions between the NTAA of the immobilized polypeptide and the nucleic acid complementary coding tags are indicated below by the following two schemes:

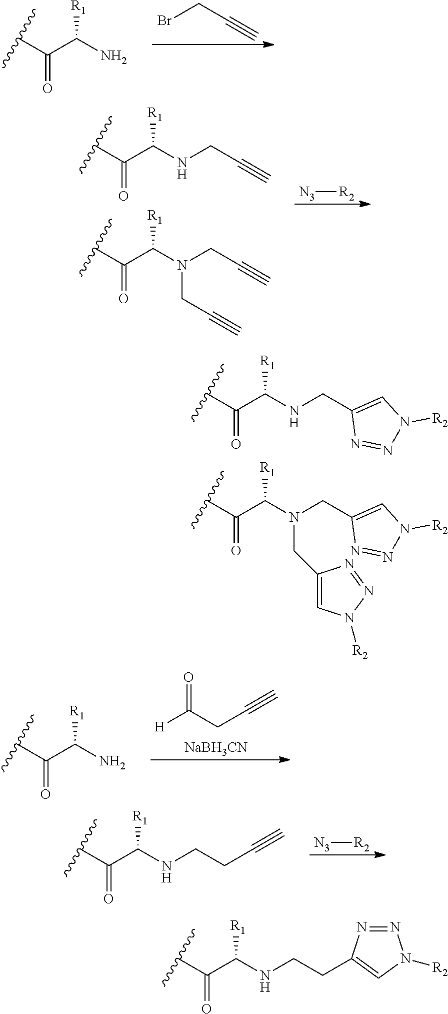

In the above schemes, R1 indicates a side chain of the NTAA of the immobilized polypeptide (amino acid residues of the immobilized polypeptide except for the NTAA residue are not shown); N3-R2 indicates an azide-modified complementary coding tag.

Exemplary conditions for the above-indicated reactions are as follows: 1) High temperature (92° C., 18 h), as disclosed in V. V. Rostovtsev, et al., Angew. Chem. Int. Ed., 2002, 41, 2596-2599; 2) 2 mol % CuSO4.5H20, 5 mol % sodium Ascorbate, H₂0:tBuOH (1:1), room temperature, as disclosed in F. Himo, et al., J. Am. Chem. Soc., 2005, 127, 210-216; 3) 2 mol % Cp*RuCl(PPh3)2, dioxane, 60° C., as disclosed in B. C. Boren, et al., J. Am. Chem. Soc., 2008, 130, 8923-8930.

In other embodiments, other reactive groups can be designed to provide covalent attachment of the moiety to the NTAA of the polypeptide.

In some embodiments, the moiety located on the complementary coding tags comprises a linker, preferably a photocleavable linker. In some embodiments, after attaching the complementary coding tag to the NTAA of the polypeptide, the photocleavable linker can be cleaved by light to release the complementary coding tag, leaving the moiety attached to the NTAA of the polypeptide. In one particular embodiment, a cleavable portion is installed between the complementary coding tag and the triazole (a product of the click reaction), or between the N-terminus of the polypeptide and the triazole.

In some embodiments, the examples of cleavable linkers include:

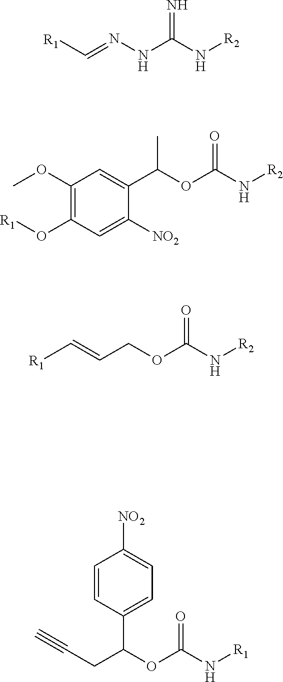

In some embodiments, photocleavable linkers with activated esters can be used. Amine reactive reagents bearing an enrichment tag and a photocleavable linker are well established for immobilization and photocleavage of target molecules. Nitroaryl, arylcarbonylmethyl, coumarin-4-ylmethyl, and arylmethyl groups and others are established photocleavable groups (Klan P, et al., Photoremovable protecting groups in chemistry and biology: reaction mechanisms and efficacy. Chem Rev. 2013 Jan 9;113(1):119-91). Amine modification rates using standard activated esters are quite high, and photocleavage efficiency is near quantitative when using an appropriate light source.

The following embodiment illustrates an exemplary workflow including an encoding reaction: a large collection of polypeptides from a proteolytic digest of a sample are conjugated independently to recording tags forming recording tag-polypeptide conjugates; then the conjugates are immobilized randomly on a porous bead at an appropriate intramolecular spacing using nucleic acid capture molecules attached to beads.

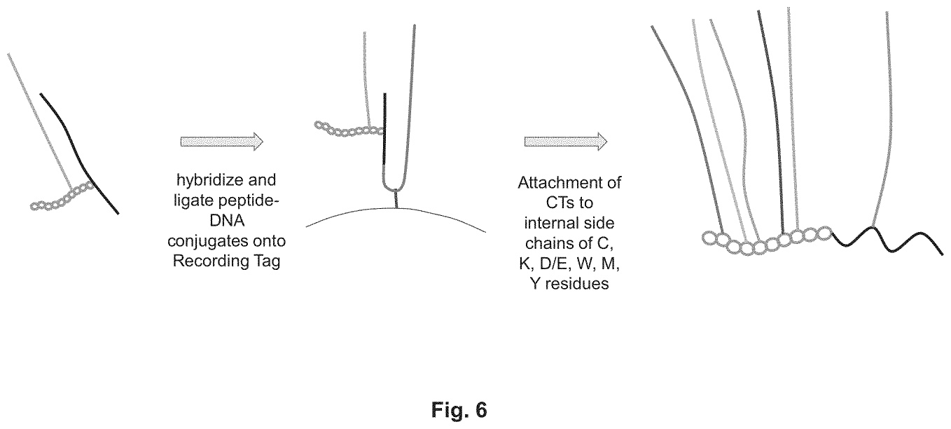

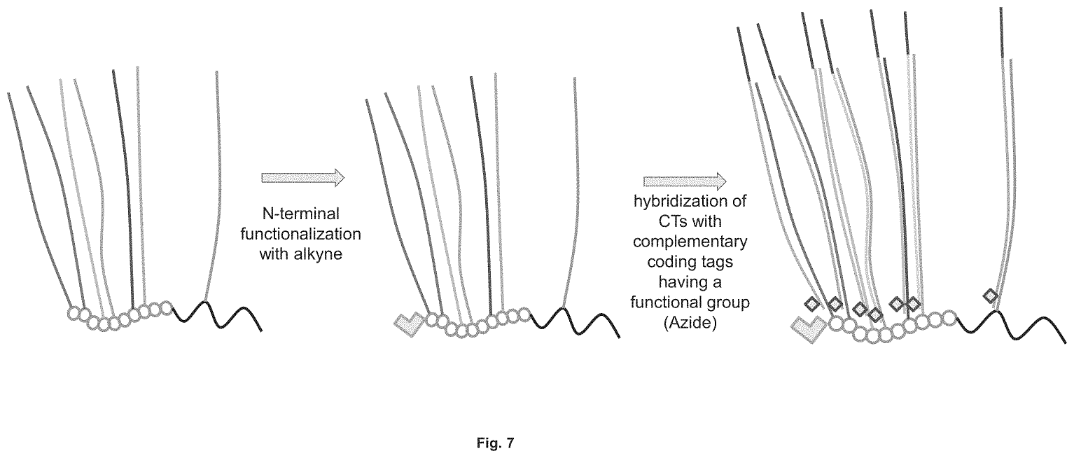

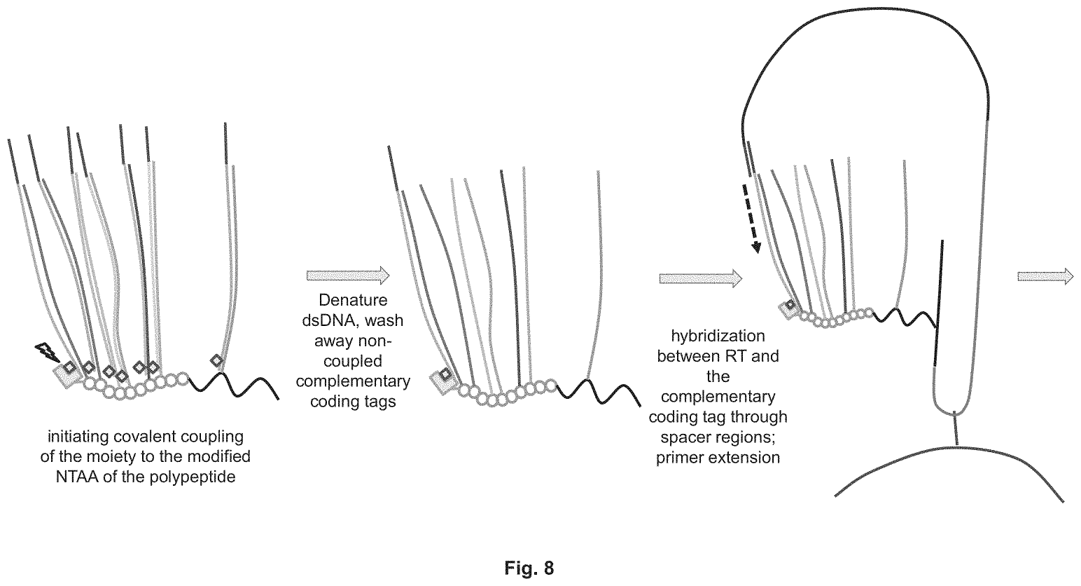

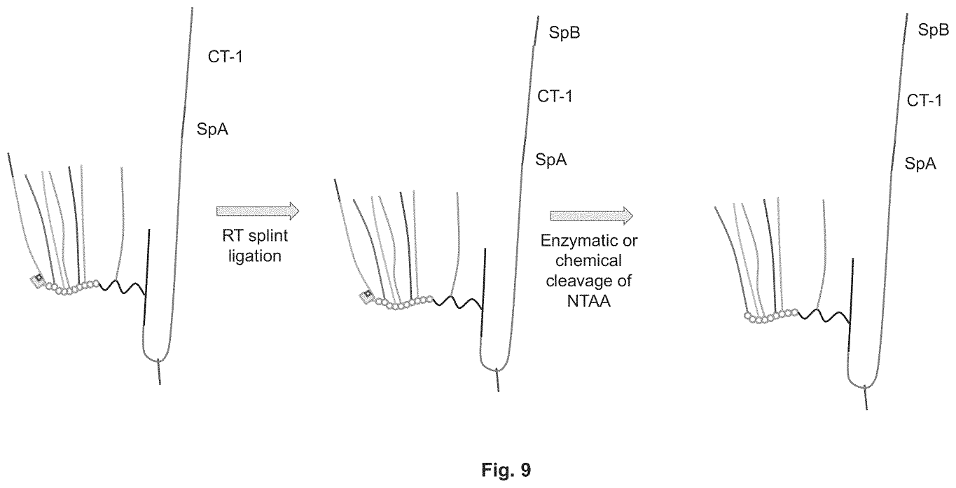

After immobilization, the immobilized recording tag-polypeptide conjugate is contacted with a plurality of short single stranded nucleic acid coding tags, wherein each coding tag of the plurality of coding tags is configured to react selectively with a specific type of amino acid residues and comprises a barcode region with identifying information regarding the specific type of amino acid residues to which the coding tag reacts selectively, thereby obtaining the polypeptide comprising coding tags attached to the specific amino acid residues (FIG. 6). Preferably, length of coding tags is 10 nucleotides or less. Different specific chemistries (disclosed above) are used sequentially to attach coding tags to 3, 4, 5, 6, 7, 8 or 9 different types of amino acid residues of the polypeptide. In the next step, the polypeptide comprising coding tags attached to the specific amino acid residues is contacted with a plurality of complementary coding tags, wherein each complementary coding tag of the plurality of complementary coding tags comprises (i) a region complementary to the barcode region of a corresponding coding tag, (ii) a first spacer region complementary to a first complementary spacer region of the recording tag, and (iii) a moiety configured, when in a close proximity, to be covalently coupled to the NTAA of the polypeptide. The recoding tag attached to the polypeptide has a region complementary to the first spacer region (a first complementary spacer region). The plurality of complementary coding tags hybridize with the coding tags attached to the specific amino acid residues of the immobilized polypeptide (FIG. 7). The moiety on the complementary coding tags has a functional group that reacts with the NTAA residue of the polypeptide, when in a close proximity (typically, less than 10 nm, and preferably, 5 nm or less), which creates a covalent linkage (FIG. 8). Only moiety located on the complementary coding tag attached to the NTAA residue will react with the reactive group of the NTAA modifier, whereas moieties located on the other complementary coding tags cannot. After reacting with the NTAA residue, the complementary coding tag attached to other than the NTAA residues are washed away by a high salt buffer. In the next step, conditions are provided to allow hybridization between the first spacer region of the complementary coding tag attached to the NTAA and the first complementary spacer region on the recording tag, followed by a primer extension reaction (FIG. 8), which allows the information from the barcode region of the complementary coding tag to get transferred to the recording tag (FIG. 9). This barcode region (CT-1 on FIG. 9) identifies the coding tag that was attached to the NTAA residue, and thus encodes information regarding identity of the NTAA residue, since each coding tag is configured to react selectively with only a specific type of amino acid residues. Thus, information regarding identity of the NTAA residue is now stored on the recording tag. In the next step, the NTAA of the polypeptide is removed, thereby exposing a new NTAA (FIG. 9). Removal is performed either chemically or enzymatically. The approaches that allow for removal of the NTAA with the attached coding tag are presented below. In a separate step, a second order complementary spacer region is added to the recording tag extended after transferring information the barcode information (This step can be performed in any order with the cleavage step). This addition is performed by splint ligation, and the second order complementary spacer region is used during the next (second) encoding cycle (FIG. 9). Additionally, in case no encoding (information transfer) occurred during the first cycle (for example, when the NTAA is not one of the specific amino acid residues, so no coding tag was attached to the NTAA), the second order complementary spacer region is necessary to mark the beginning of the second cycle, which allows for identification of correct spacing between all the identifiable amino acid residues (amino acid residues of a specific type, e.g., lysine, arginine, aspartate, glutamate, histidine, cysteine, serine, methionine, tryptophan and tyrosine).

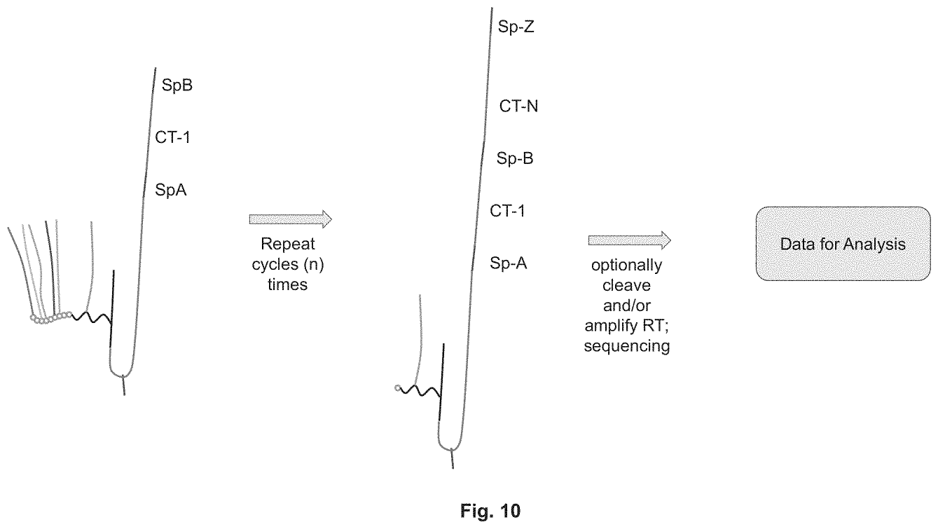

Finally, after the NTAA cleavage and the second order complementary spacer region addition, the next cycle starts by contacting the polypeptide comprising the coding tags attached to the specific amino acid residues with a second plurality of complementary coding tags, wherein each complementary coding tag of the plurality of complementary coding tags comprises (i) a region complementary to the barcode region of a corresponding coding tag, (ii) a second spacer region complementary to a second complementary spacer region of the recording tag, and (iii) a moiety configured, when in a close proximity, to be covalently coupled to the NTAA of the polypeptide. In the second cycle, each of the complementary coding tags contains a second spacer region, which is different from the first spacer region used in the first cycle (a cycle specific spacer region is used) (FIG. 10). After that, steps of covalently coupling the moiety to the NTAA of the polypeptide; removing (washing away) complementary coding tags that are not covalently coupled to the NTAA of the polypeptide; transferring information of the barcode region from the complementary coding tag covalently coupled to the NTAA to the recording tag; removing the NTAA of the polypeptide, thereby exposing a new NTAA; and adding a third order complementary spacer region to the recording tag extended after information transfer are repeated. After the second cycle, the extended recording tag contains barcode regions from the first and the second amino acid residues of the polypeptide, if the first and the second amino acid residues are identifiable (specific) amino acid residues. Thus, by sequencing the extended recording tag, the identifying information regarding the first and the second amino acid residues can be obtained. Further cycles (third, fourth, and so on) are employed to encode identifying information about all the identifiable (specific) amino acid residues of the polypeptide into the recording tag. In the case when a non-identifiable amino acid residue is present at the N-terminus of the polypeptide, this residue will not have an attached coding tag, so not encoding (information transfer) can occur at this cycle, and the recording tag will be extended only to the next complementary spacer region. After performing n described cycles, the recording tag will contain n complementary spacer regions and also barcode regions between them for each cycle with encoding. Thus, by decoding information from the extended recording tag, both identity of the specific amino acid residues and correct spacing between them can be determined. Based on this information, the polypeptide can be identified.

With the ability to determine identity and correct order of identifiable amino acid residues, the disclosed methods are capable of producing unique patterns sufficiently reflective of the polypeptide sequences to allow identification of proteins from specific proteomes, such as human proteomes.

The resulting data obtained after extended recording tag sequencing do not or may not provide a complete polypeptide sequence, but rather a pattern (a signature) of specific amino acid residues (e.g. X-K-X-C-Y-X-M-X-K- . . .) that can be searched against the known proteome sequences in order to identify the immobilized polypeptide. If only a few identifiable (specific) amino acid residues is used for reaction with coding tags, the obtained patterns may match to several polypeptide sequences in the proteome, and thus may not always be sufficiently information-rich to unambiguously identify the polypeptide, although by combining information from multiple peptides belonging to the same protein, the unique identification of proteins can be substantially higher.

In some embodiments, the polypeptide will be identified by comparison of the generated pattern with other patterns generated computationally using a database of possible protein sequences from the organism being analyzed (e.g., if a human sample is analyzed, then a human proteome database is used to generate theoretical patterns for comparison). If sample potentially contains a proteomic mixture of different species, then their proteomes can be combined before extracting theoretical polypeptide patterns for comparison. In other embodiments, genomic databases can be utilized to extract theoretical polypeptide patterns from coding regions of the genome(s).

Computer simulations have shown that relatively simple labeling schemes of specific amino acid residues are sufficient to identify most proteins in the human proteome. Employing only 1 to 4 amino acid specific fluorescent labels can yield patterns capable of uniquely identifying at least one peptide from most of the known human proteins (Swaminathan J, Boulgakov A A, Marcotte E M. A theoretical justification for single molecule peptide sequencing. PLoS Comput Biol. 2015 Feb. 25;11(2):e1004080). Increasing the number of distinct label types improves identification up to 80% within only 20 experimental cycles even when only Cys-containing peptides are sequenced; near total proteome coverage is theoretically achievable when cyanogen bromide generated peptides are anchored by their C-termini and labeled by a combination of four different fluorophores (see Swaminathan J, Boulgakov A A, Marcotte E M. PLoS Comput Biol. 2015 Feb. 25;11(2):e1004080). Thus, based on these calculations, a plurality of coding tags targeting four different specific amino acid residues is sufficient for identification of a polypeptide from the human proteome after 20 encoding cycles.

Barcode information of the coding tag that targets a specific type of amino acid residues may be transferred to the recording tag using a variety of methods. In certain embodiments, barcode information of the coding tag is transferred to the recording tag via primer extension performed by a DNA polymerase having 5′-to-3′ polymerization activity and having substantially reduced 3′-to-5′ exonuclease activity. A complementary spacer region on the 3′-terminus of the recording tag or the extended recording tag (generated after the first encoding cycle) anneals with spacer region on the 3′ terminus of the complementary coding tag attached to the NTAA, and a polymerase (preferably, a polymerase with a high strand-displacement activity) extends the recording tag sequence, using the annealed complementary coding tag as a template.

In a preferred embodiment, a DNA polymerase that is used for primer extension possesses high strand displacement activity and has limited or is devoid of 3′-5 exonuclease activity. Several of many examples of such polymerases include Klenow exo-(Klenow fragment of DNA Pol 1), T4 DNA polymerase exo-, T7 DNA polymerase exo (Sequenase 2.0), Pfu exo-, Vent exo-, Deep Vent exo-, Bst DNA polymerase large fragment exo-, Bca Pol, 9 N Pol, and Phi29 Pol exo-. In a preferred embodiment, the DNA polymerase is active at room temperature and up to 45° C. In another embodiment, a “warm start” version of a thermophilic polymerase is employed such that the polymerase is activated and is used at about 40-50° C. An exemplary warm start polymerase is Bst 2.0 Warm Start DNA Polymerase (New England Biolabs).

In another embodiment, optimal polymerase extension buffers are comprised of 40-120 mM buffering agent such as Tris-Acetate, Tris-HCl, HEPES, etc. at a pH of 6-9.

Barcode information may also be transferred from the coding tag to the recording tag via ligation. Ligation may be an enzymatic ligation reaction or a chemical ligation reaction. For example, a splint ligation can be accomplished by using hybridization of a “recording helper” sequence with an arm on the coding tag. The annealed complement sequences are chemically ligated using standard chemical ligation or “click chemistry”.

In another embodiment, transfer of PNAs can be accomplished with chemical ligation using published techniques. The structure of PNA is such that it has a 5′ N-terminal amine group and an unreactive 3′ C-terminal amide. Chemical ligation of PNA requires that the termini be modified to be chemically active. This is typically done by derivatizing the 5′ N-terminus with a cysteinyl moiety and the 3′ C-terminus with a thioester moiety. Such modified PNAs easily couple using standard native chemical ligation conditions (Roloff et al., (2013) Bioorgan. Med. Chem. 21:3458-3464).

In some embodiments, to minimize non-specific (or repulsive) interaction of the coding tags attached to the specific amino acid residues of the immobilized polypeptide, competitor polymers (e.g., positively charged polymers) can be added to encoding reactions to minimize non-specific interactions. Excess competitor polymers are washed from the reaction prior to primer extension, which effectively dissociates the annealed competitor polymers from the coding tags or the recording tag, especially when exposed to slightly elevated temperatures (e.g., 30-50° C.).

The extended recording tag is a nucleic acid molecule or sequenceable polymer molecule (see, e.g., Niu et al., 2013, Nat. Chem. 5:282-292; Roy et al., 2015, Nat. Commun. 6:7237) that comprises identifying information for a associated polypeptide. In some cases, an extended recording tag will experience a “missed” encoding cycle, e.g., when the moiety of a complementary coding tag fails to covalently couple to the NTAA residue of the polypeptide or the modified NTAA residue of the polypeptide, because the coding tag was not attached to the NTAA residue or because the primer extension reaction failed. Additionally, transfer of information from the coding tag may be incomplete or less than 100% accurate, e.g., because a coding tag was damaged or defective, because errors were introduced in the primer extension reaction). Thus, an extended recording tag may represent 100%, or up to 95%, 90%, 75%, 50%, 30%, or any subrange thereof, of encoding events that have occurred on its associated polypeptide. Moreover, the coding tag information present in the extended recording tag may have at least 30%, 50%, 75%, 90%, 95%, or 100% identity the corresponding coding tags. In preferred embodiments, an extended recording tag associated with the immobilized polypeptide comprises information from multiple coding tags attached to the specific amino acid residues of the immobilized polypeptide representing multiple, successive encoding events. In these embodiments, a single, concatenated extended recording tag associated with the immobilized polypeptide can be representative of a single polypeptide. As referred to herein, transfer of coding tag information to the recording tag associated with the immobilized polypeptide also includes transfer to an extended recording tag as would occur in methods involving multiple, successive encoding events.

In certain embodiments, a recording tag comprises an optional unique molecular identifier (UMI), which provides a unique identifier tag for each polypeptide to which the UMI is associated with. A UMI can be about 3 to about 40 bases, about 3 to about 20 bases, or about 3 to about 10 bases, or about 3 to about 8 bases in length. A UMI can be used to de-convolute sequencing data from a plurality of extended recording tags to identify sequence reads from individual polypeptides. In some embodiments, within a library of polypeptides, each polypeptide is associated with a single recording tag, with each recording tag comprising a unique UMI. In other embodiments, multiple copies of a recording tag are associated with a single polypeptide, with each copy of the recording tag comprising the same UMI.

In certain embodiments, a recording tag comprises a universal priming site, e.g., a forward or 5′ universal priming site. A universal priming site is a nucleic acid sequence that may be used for priming a library amplification reaction and/or for sequencing. A universal priming site may include, but is not limited to, a priming site for PCR amplification, flow cell adaptor sequences that anneal to complementary oligonucleotides on flow cell surfaces (e.g., Illumina next generation sequencing), a sequencing priming site, or a combination thereof. A universal priming site can be about 10 bases to about 60 bases. In some embodiments, a universal priming site comprises an Illumina P5 primer (5′-AATGATACGGCGACCACCGA-3′—SEQ ID NO:1) or an Illumina P7 primer (5′-CAAGCAGAAGACGGCATACGAGAT—3′- SEQ ID NO:2).

In some examples, the labeling of the polypeptide with a recording tag is performed using standard amine coupling chemistries. In a particular embodiment, the recording tag can comprise a reactive moiety (e.g., for conjugation to a solid surface, a multifunctional linker, or a polypeptide), a linker, a universal priming sequence, a barcode, an optional UMI, and a spacer (Sp) sequence for facilitating information transfer to/from a coding tag. In another embodiment, the protein is labeled with a universal DNA tag prior to proteinase digestion into peptides. The universal DNA tags on the labeled peptides from the digest can then be converted into an informative and effective recording tag. A universal DNA tag comprises a short sequence of nucleotides that are used to label a polypeptide macromolecule and can be used as point of attachment. For example, a recording tag may comprise at its terminus a sequence complementary to the universal DNA tag. In certain embodiments, a universal DNA tag is a universal priming sequence. Upon hybridization of the universal DNA tags on the labeled protein to complementary sequence in recording tags (e.g., bound to beads), the annealed universal DNA tag may be extended via primer extension, transferring the recording tag information to the DNA tagged polypeptide.

The recording tags may comprise a reactive moiety for a cognate reactive moiety present on the target polypeptide (e.g., click chemistry labeling, photoaffinity labeling). For example, recording tags may comprise an azide moiety for interacting with alkyne-derivatized proteins, or recording tags may comprise a benzophenone for interacting with native polypeptide. Upon binding of the target polypeptide by the coding tags, the recording tag and target polypeptide are coupled via their corresponding reactive moieties. In some embodiments, other types of linkages besides nucleic acid hybridization can be used to link the recording tag to a polypeptide. A suitable linker can be attached to various positions of the recording tag, such as the 3′ end, at an internal position, or within the linker attached to the 5′ end of the recording tag.

In some embodiments, the extended recording tag generated from performing the provided methods comprises information transferred from multiple coding tags. In some embodiments, the extended recording tags are amplified (or a portion thereof) prior to determining at least the sequence of the coding tag(s) in the extended recording tag. In some embodiments, the extended recording tags (or a portion thereof) are released prior to determining at least the sequence of the coding tag(s) in the extended recording tag.

The length of the final extended recording tag generated by the methods described herein is dependent upon multiple factors, including the length of the coding tag(s) (e.g., barcode and spacer), and optionally including any unique molecular identifier, spacer, universal priming site, barcode, or combinations thereof. After transfer of the final tag information to the extended nucleic acid (e.g., from any coding tags), the tag can be capped by addition of a universal reverse priming site via ligation, primer extension or other methods known in the art. In some embodiments, the universal forward priming site in the nucleic acid (e.g., on the recording tag) is compatible with the universal reverse priming site that is appended to the final extended nucleic acid. In some embodiments, a universal reverse priming site is an Illumina P7 primer (5′-CAAGCAGAAGACGGCATACGAGAT—3′-SEQ ID NO:2) or an Illumina P5 primer (5′-AATGATACGGCGACCACCGA-3′—SEQ ID NO:1). The sense or antisense P7 may be appended, depending on strand sense of the nucleic acid to which the identifying information from the coding tag is transferred to. An extended nucleic acid library can be cleaved or amplified directly from the support (e.g., beads) and used in traditional next generation sequencing assays and protocols.

Extended nucleic acids recording tags can be processed and analysed using a variety of nucleic acid sequencing methods. In some embodiments, extended recording tags containing the information from one or more coding tags and any other nucleic acid components are processed and analyzed. In some embodiments, the collection of extended recording tags can be concatenated. In some embodiments, the extended recording tag can be amplified prior to determining the sequence.

Examples of sequencing methods include, but are not limited to, chain termination sequencing (Sanger sequencing); next generation sequencing methods, such as sequencing by synthesis, sequencing by ligation, sequencing by hybridization, polony sequencing, ion semiconductor sequencing, and pyrosequencing; and third generation sequencing methods, such as single molecule real time sequencing, nanopore-based sequencing, duplex interrupted sequencing, and direct imaging of DNA using advanced microscopy.

Suitable sequencing methods for use in the invention include, but are not limited to, sequencing by hybridization, sequencing by synthesis technology (e.g., HiSeg™ and Solexa™, Illumina), SMRT™ (Single Molecule Real Time) technology (Pacific Biosciences), true single molecule sequencing (e.g., HeliScope™, Helicos Biosciences), massively parallel next generation sequencing (e.g., SOLiD™, Applied Biosciences; Solexa and HiSeg™, Illumina), massively parallel semiconductor sequencing (e.g., Ion Torrent), pyrosequencing technology (e.g., GS FLX and GS Junior Systems, Roche/454), nanopore sequence (e.g., Oxford Nanopore Technologies).

In some embodiments, a library of nucleic acids (e.g., extended nucleic acids) is concatenated by ligation or end-complementary PCR to create a long DNA molecule comprising multiple different extended recorder tags or extended coding tags, respectively (Du et al., (2003) BioTechniques 35:66-72; Muecke et al., (2008) Structure 16:837-841; U.S. Pat. No. 5,834,252). This embodiment is preferable for nanopore sequencing in which long strands of DNA are analyzed by the nanopore sequencing device. In some embodiments, direct single molecule analysis is performed on the nucleic acids (e.g., extended nucleic acids) (see, e.g., Harris et al., (2008) Science 320:106-109). The nucleic acids (e.g., extended nucleic acids) can be analysed directly on the support, such as a flow cell or beads that are compatible for loading onto a flow cell surface (optionally microcell patterned), wherein the flow cell or beads can integrate with a single molecule sequencer or a single molecule decoding instrument. For single molecule decoding, hybridization of several rounds of pooled fluorescently-labelled decoding oligonucleotides (Gunderson et al., (2004) Genome Res. 14:970-7) can be used to ascertain both the identity and order of the coding tags within the extended nucleic acids (e.g., on the recording tag). Following sequencing of the nucleic acid libraries (e.g., of extended nucleic acids), the resulting sequences can be collapsed by their UMIs if used and then associated to their corresponding polypeptides and aligned to the totality of the proteome. Resulting sequences can also be collapsed by their compartment tags and associated to their corresponding compartmental proteome, which in a particular embodiment contains only a single or a very limited number of protein molecules. Both protein identification and quantification can easily be derived from this digital peptide information.

The methods disclosed herein can be used for analysis, including identification, detection, quantitation and/or sequencing, of a plurality of polypeptides simultaneously (multiplexing). Multiplexing as used herein refers to analysis of a plurality of polypeptides in the same assay. The plurality of polypeptides can be derived from the same sample or different samples. The plurality of polypeptides can be derived from the same subject or different subjects. The plurality of polypeptides that are analyzed can be different polypeptides, or the same polypeptide derived from different samples. A plurality of polypeptides includes 10 or more polypeptides, 50 or more polypeptides, 100 or more polypeptides, 500 or more polypeptides, 1,000 or more polypeptides, 5,000 or more polypeptides, 10,000 or more polypeptides, 100,000 or more polypeptides, or 1,000,000 or more polypeptides.

In the methods disclosed herein, the recording tag extended at step (j) is analyzed by a nucleic acid sequencing method, and information regarding the specific amino acid residues of the polypeptide is obtained, which will lead to the polypeptide identification. Information regarding the specific amino acid residues of the polypeptide is obtained by analyzing sequences of the barcode regions present in the extended recording tag after transfer of information, and decoding identifying information present in the barcode regions to identify coding tags attached to the specific amino acid residues of the immobilized polypeptide. In the preferred embodiments, the recording tag extended after the last encoding (transfer of information) cycle is sequenced. In some embodiments, the extended recording tag may be sequenced after each encoding cycle or after selected encoding cycles.

In some embodiments, the analysis step (k) comprises bioinformatically matching the obtained information regarding the specific amino acid residues of the polypeptide with corresponding information extracted from a genomic or proteomic database. Under ideal scenario or in preferred embodiment, at each encoding cycle, either the barcode information is accumulated on the extended recording tag followed by addition of the spacer region, or just the spacer region is added on the extended recording tag, when there is no coding tag attached to the NTAA residue in the current cycle (thus, no complementary coding tag is attached to the NTAA at this cycle). Analyzing sequence of the extended recording tag after completion of n encoding cycles will provide information regarding n consecutive amino acid residues of the immobilized polypeptide: whether there are specific amino acid residues to which coding tags are attached and the order of this specific residues. One can use genomic or proteomic database containing information regarding proteins potentially present in the analyzed sample, and create in silico patterns for the whole proteome (or part of the proteome) which indicate the presence and order of the specific amino acid residues. Then, information extracted from the extended recording tag can be matched with these patterns in order to identify the immobilized polypeptide with certain probability. Even in the presence of errors that can occur during the encoding cycles (such as missing an encoding event due to failure to transfer the barcode information), only limited number of specific amino acid residues will be sufficient to identify the immobilized polypeptide with high probability given that both type and order of the specific amino acid residues can be obtained during the analysis of the extended recording tag. Exemplary calculations of minimal number of the specific amino acid residues required to achieve a certain probability in polypeptide identification for a particular proteome were previously published (Swaminathan J, et al., A theoretical justification for single molecule peptide sequencing. PLoS Comput Biol. 2015 Feb. 25;11(2):e1004080).

In some embodiments, the described analysis and polypeptide identification are performed in parallel for multiple analyzed polypeptides. Following sequencing of the extended recording tags, the resulting sequences can be collapsed by their UMIs and then associated to their corresponding polypeptides and aligned to the totality of the polypeptides in the cell. Resulting sequences can also be collapsed by their compartment tags and associated to their corresponding compartmental proteome, which in a particular embodiment contains only a single or a very limited number of protein molecules (if prior compartmentalization is utilized during sample preparation).

In some embodiments, a coding tag from the plurality of coding tags is configured to react selectively with a post-translationally modified amino acid residue. In one example, the specific amino acid residue to which the coding tag reacts selectively is a phosphoserine or phosphothreonine. In this embodiment, the barcode region of the coding tag contains identifying information regarding a phosphoserine or phosphothreonine, so post-translationally modified polypeptides can be identified by the described methods by allowing information transfer to the recording tag as described above. Any post-translational modification of amino acid residues can be identified, as long as a specific chemistry can be designed to selectively target these modified amino acid residues.

In some embodiments, removing complementary coding tags that are not covalently coupled to the NTAA of the polypeptide is achieved by washing the immobilized polypeptides with high salt buffers, such as, for examples, phosphate or MOPS buffer supplemented with 0.3M-1M NaCl. High salt buffers can efficiently disrupt nucleic acid hybridization, removing non-coupled complementary coding tags.

In preferred embodiments, after transferring identifying information of the barcode region or the region complementary to the barcode region from complementary coding tag covalently coupled to the NTAA of the polypeptide to the recording tag, the NTAA of the polypeptide is removed to expose a new NTAA of the polypeptide. This starts the next encoding cycle, where the polypeptide comprising coding tags attached to the specific amino acid residues is contacted with another plurality of complementary coding tags and steps (c)-(h) are repeated one or more times by replacing at step (c) the first spacer region of the complementary coding tags with a second or higher order spacer region complementary to the second or higher order complementary spacer region of the recording tag, and by replacing at step (h) the second complementary spacer region with a third or higher order complementary spacer region. The NTAA of the immobilized polypeptide can removed by either chemical or enzymatic method.

Exemplary methods of chemical NTAA removal from the immobilized polypeptide are disclosed in U.S. Pat. No. 9625469 B2, and in the following published patent applications: US 2020/0348307 A1, US 2020/0400677 A1, US 2020/0217853 A1, WO 2020/223133 A1, and in U.S. provisional application Ser. No. 17/606,759.

In some embodiments, the NTAA of the polypeptide is removed by the following method: (a) functionalizing the N-terminal amino acid (NTAA) of the polypeptide with a chemical reagent, wherein the chemical reagent is either:

(i) a compound of Formula (AA):

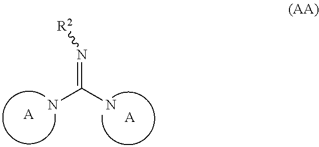

wherein: R2 is H or R4;

R4 is C1-6 alkyl, which is optionally substituted with one or two members selected from halo, C1-3 alkyl, C1-3 alkoxy, C1-3 haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C1-3 alkyl, C1-3 alkoxy, C1-3 haloalkyl, NO2, CN, COOR″, and CON(R″)2,

where each R″ is independently H or C1-3 alkyl;

each ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C1-4 alkyl, C1-4 alkoxy, —OH, halo, C1-4 haloalkyl, NO2, COOR, CONR2, —SO2R*, —NR2, phenyl, and 5-6 membered heteroaryl;

wherein each R is independently selected from H and C1-3 alkyl optionally substituted with OH, OR*, —NH2, —NHR*, or —NR*2; and

each R* is C1-3 alkyl, optionally substituted with OH, oxo, C1-2 alkoxy, or CN;

wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C1-2 alkyl, OH, oxo, C1-2 alkoxy, or CN; or

(ii) a compound of the formula R3-NCS;

wherein R3 is H or an optionally substituted group selected from phenyl, 5-membered heteroaryl, 6-membered heteroaryl, C1-3 haloalkyl, and C1-6 alkyl,

wherein the optional substituents are one to three members selected from halo, —OH, C1-3 alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and ₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;

where each R′ is independently H or C₁₋₃ alkyl;

wherein two R′ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;

to provide an initial NTAA functionalized polypeptide;

optionally treating the initial NTAA functionalized polypeptide with an amine of Formula R²-NH₂ or with a diheteronucleophile to form a secondary NTAA functionalized polypeptide; and

(b) treating the initial NTAA functionalized polypeptide or the secondary NTAA functionalized polypeptide with a suitable medium to cleave the NTAA, thereby removing the NTAA of the polypeptide.

In some preferred embodiments, the suitable medium has pH between about 5 and about 9, and optionally includes a hydroxide, carbonate, phosphate, sulfate or amine.

In some preferred embodiments, treating the initial NTAA functionalized polypeptide or the secondary NTAA functionalized polypeptide with the suitable medium occurs at a temperature between about 40° C. and about 95° C.

In some embodiments, a method to remove an N-terminal amino acid residue from a peptidic compound of Formula (I)

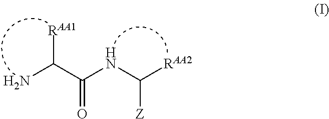

is used, wherein the method comprises:

(1) converting the peptidic compound to a guanidinyl derivative of Formula (II):

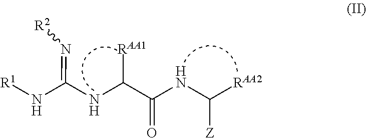

or a tautomer thereof; and

(2) contacting the guanidinyl derivative with a suitable medium to produce a compound of Formula (III)

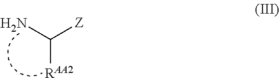

wherein:

R¹ is R⁶, NHR³, —NHC(O)—R³, or —NH—SO₂—R³

R² is H or R⁴;

R³ is H or R⁶, wherein R⁶ is an optionally substituted group selected from phenyl, 5-membered heteroaryl, 6-membered heteroaryl, C₁₋₃ haloalkyl, and C₁₋₆ alkyl,

wherein optional substituents of the optionally substituted group are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
where each R′ is independently H or C₁₋₃ alkyl;

R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,
where each R″ is independently H or C₁₋₃ alkyl;
and wherein two R′ or two R″ on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OCH₃, CH₃, oxo, NH₂, NHCH₃ and N(CH₃)₂;

R^{AA1} and R^{AA2} are each independently selected amino acid side chains;
and the dashed semi-circle connecting R^{AA1} and/or R^{AA2} to the nearest N atom indicates that R^{AA1} and/or R^{AA2} can optionally cyclize onto the designated N atom; and

Z is a polypeptide that is attached to solid support.

In some embodiments, the compound of Formula (I) is of the formula (IA):

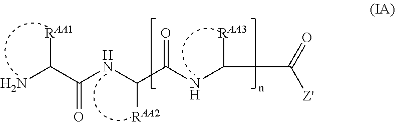

and the compound of Formula (III) is a compound of the formula (IIIA):

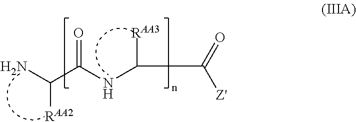

where n is an integer from 1 to 1000;

R^{AA1} and R^{AA2} are as defined in claim 1;

the dashed semi-circle connecting R^{AA1} and R^{AA2} and R^{AA3} to the adjacent N atom indicates that R^{AA1} and/or R^{AA2} and/or R^{AA3} can optionally cyclize onto the designated adjacent N atom; and

each R^{AA3} is independently selected from amino acid side chains, including natural and non-natural amino acids;
and Z′ is OH or NH₂, or Z′ is O or N that is attached to a carrier or solid support.

In some embodiments, the guanidinyl derivative of Formula (II) is produced by converting the peptidic compound of Formula (I) to a compound of the formula (IV):

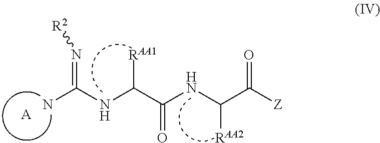

wherein ring A is a 5-6 membered heteroaryl ring containing up to three N atoms as ring members, optionally fused to an additional 5-6 membered heteroaryl or phenyl ring, and wherein the 5-6 membered heteroaryl ring and optional additional 5-6 membered heteroaryl or phenyl ring are each optionally substituted with up to four groups selected from C₁₋₄ alkyl, C₁₋₄ alkoxy, —OH, halo, C₁₋₄haloalkyl, NO₂, COOR, CONR₂, —SO₂R*, and —NR₂;
wherein each R is independently selected from H and C₁₋₃ alkyl, optionally substituted with OH, OR*, —NH₂, and —NR*₂; and
each R* is C₁₋₃ alkyl, optionally substituted with OH, C₁₋₂ alkoxy, —NH₂, or CN; or a salt thereof;

wherein two R or two R* on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OCH₃, CH₃, oxo, NH₂, NHCH₃ and N(CH₃)₂;
the dashed semi-circle connecting R^{AA1} and R^{AA2} to the nearest N atom indicates that R^{AA1} and/or R^{AA2} optionally cyclize onto the designated N atom;
then contacting this compound with a diheteronucleophile, optionally in the presence of a buffer, to produce the compound of Formula (II).

In some embodiments, the peptidic compound of Formula (I) is converted to a compound of Formula (IV) by contacting the compound of Formula (I) with a compound of the formula:

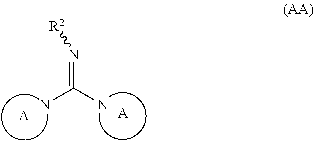

wherein:

R² is H or R⁴;

R⁴ is C₁₋₆ alkyl, which is optionally substituted with one or two members selected from halo, C₁₋₃ alkyl, C₁₋₃ alkoxy, C₁₋₃ haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C₁₋₃ alkyl, C₁₋₃ alkoxy, C₁₋₃ haloalkyl, NO₂, CN, COOR″, and CON(R″)₂,

where each R″ is independently H or C1-3 alkyl;
ring A a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C₁₋₄ alkyl, C₁₋₄ alkoxy, —OH, halo, C₁₋₄ haloalkyl, NO₂, COOR, CONR₂, —SO₂R*, —NR₂, B(OR)₂, Bpin (boranyl pinacolate), phenyl, and 5-6 membered heteroaryl;
wherein each R is independently selected from H and C₁₋₃ alkyl optionally substituted with OH, OR*, —NH₂, —NHR*, or —NR*₂; and
each R* is C₁₋₃ alkyl, optionally substituted with OH, oxo, C₁₋₂ alkoxy, or CN;
wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, and CN;
to form the compound of Formula (IV).

In some embodiments, the ring A is selected from:

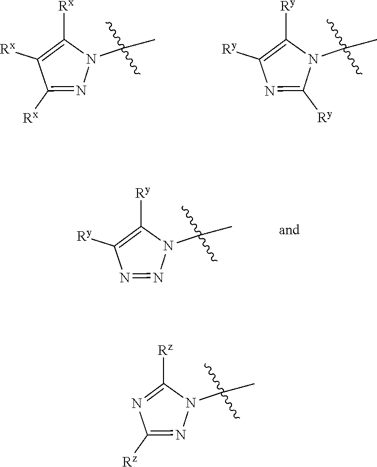

wherein:

each R^{x}, R^{y} and R^{z} is independently selected from H, halo, C₁₋₂ alkyl, C₁₋₂ haloalkyl, NO₂, SO₂(C₁₋₂ alkyl), COOR^{#}, C(O)N(R^{#})₂, and phenyl optionally substituted with one or two groups selected from halo, C₁₋₂ alkyl, C₁₋₂ haloalkyl, NO₂, SO₂(C₁₋₂ alkyl), COOR^{#}, and C(O)N(R^{#})₂,
and two R^{x}, R^{y} or R^{z} on adjacent atoms of a ring can optionally be taken together to form a phenyl group, 5-membered heteroaryl group, or 6-membered heteroaryl group fused to the ring, and the fused phenyl, 5-membered heteroaryl, or 6-membered heteroaryl group can optionally be substituted with one or two groups selected from halo, C₁₋₂ alkyl, C₁₋₂ haloalkyl, NO₂, SO₂(C₁₋₂ alkyl), COOR^{#}, and C(O)N(R^{#})₂;
wherein each R^{#} is independently H or C₁₋₂ alkyl; and wherein two R# on the same nitrogen can optionally be taken together to form a 4-7 membered heterocycle optionally containing an additional heteroatom selected from N, O and S as a ring member, wherein the 4-7 membered heterocycle is optionally substituted with one or two groups selected from halo, OH, OCH₃, CH₃, oxo, NH₂, NHCH₃ and N(CH₃)₂;
or a salt thereof.

In some embodiments, the suitable medium in step (2) of the method comprises diheteronucleophile, wherein the diheteronucleophile is selected from the following compounds:

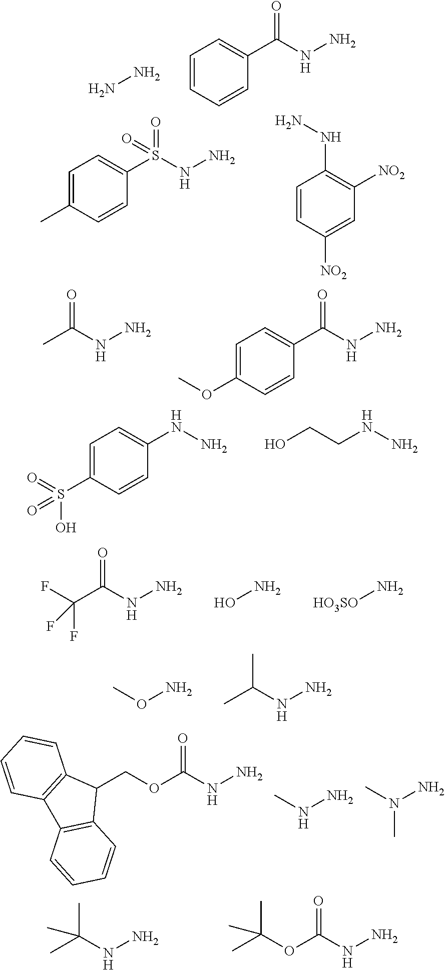

As used herein, the term “alkyl” refers to and includes saturated linear and branched univalent hydrocarbon structures and combination thereof, having the number of carbon atoms designated (i.e., C₁-C₁₀ or C₁₋₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”), unless otherwise specified Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

As used herein, “alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

The term “aminoalkyl” refers to an alkyl group that is substituted with one or more —NH₂ groups. In certain embodiments, an aminoalkyl group is substituted with one, two, three, four, five or more —NH₂ groups. An aminoalkyl group may optionally be substituted with one or more additional substituents as described herein.

As used herein, “aryl” or “Ar” refers to an unsaturated aromatic carbocyclic group having a single ring (e.g., phenyl) or multiple condensed rings (e.g., naphthyl or anthryl) which condensed rings may or may not be aromatic. In one variation, the aryl group contains from 6 to 14 annular carbon atoms. An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position. In some embodiments, phenyl is a preferred aryl group.

As used herein, the term “arylalkyl” refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

As used herein, the term “cycloalkyl” refers to and includes cyclic univalent hydrocarbon structures, which may be fully saturated, mono- or polyunsaturated, but which are non-aromatic, having the number of carbon atoms designated (e.g., Ci-Cio means one to ten carbons). Cycloalkyl can consist of one ring, such as cyclohexyl, or multiple rings, such as adamantly, but excludes aryl groups. A cycloalkyl comprising more than one ring may be fused, spiro or bridged, or combinations thereof. In some embodiments, the cycloalkyl is a cyclic hydrocarbon having from 3 to 13 annular carbon atoms. In some embodiments, the cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

As used herein, the “halogen” represents chlorine, fluorine, bromine, or iodine. The term “halo” represents chloro, fluoro, bromo, or iodo.

The term “haloalkyl” refers to an alkyl group as described above, wherein one or more hydrogen atoms on the alkyl group have been replaced by a halo group. Examples of such groups include, without limitation, fluoroalkyl groups, such as fluoroethyl, trifluoromethyl, difluoromethyl, trifluoroethyl and the like.

As used herein, the term “heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. It is understood that the selection and order of heteroatoms in a heteroaryl ring must conform to standard valence requirements and provide an aromatic ring character, and also must provide a ring that is sufficiently stable for use in the reactions described herein. Typically, a heteroaryl ring has 5-6 ring atoms and 1-4 heteroatoms, which are selected from N, O and S unless otherwise specified; and a bicyclic heteroaryl group contains two 5-6 membered rings that share one bond and contain at least one heteroatom and up to 5 heteroatoms selected from N, O and S as ring members. A heteroaryl group can be attached to the remainder of the molecule at an annular carbon or at an annular heteroatom, in which case the heteroatom is typically nitrogen. Heteroaryl groups may contain additional fused rings (e.g., from 1 to 3 rings), including additionally fused aryl, heteroaryl, cycloalkyl, and/or heterocyclyl rings. Examples of heteroaryl groups include, but are not limited to, pyrazolyl, imidazolyl, triazolyl, pyrrolyl, pyridyl, pyrimidyl, pyrazinyl, pyridazinyl, triazinyl, thiophenyl, furanyl, thiazolyl, and the like.

As used herein, the term “heterocycle”, “heterocyclic”, or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, tetrahydrothiophenyl, 2,3 -dihydrobenzo[b]thiophen-2-yl, 4-amino-2-oxopyrimidin-1(2H)-yl, and the like.

The term “substituted” means that the specified group or moiety bears one or more substituents in place of a hydrogen atom of the unsubstituted group, including, but not limited to, substituents such as alkoxy, acyl, acyloxy, carbonylalkoxy, acylamino, amino, aminoacyl, aminocarbonylamino, aminocarbonyloxy, cycloalkyl, cycloalkenyl, aryl, heteroaryl, aryloxy, cyano, azido, halo, hydroxyl, nitro, carboxyl, thiol, thioalkyl, cycloalkyl, cycloalkenyl, alkyl, alkenyl, alkynyl, heterocyclyl, aralkyl, aminosulfonyl, sulfonylamino, sulfonyl, oxo, carbonylalkylenealkoxy and the like. The term “unsubstituted” means that the specified group bears no substituents. The term “optionally substituted” means that the specified group is unsubstituted or substituted by one or more substituents and thus includes both substituted and unsubstituted versions of the group. Where the term “substituted” is used to describe a structural system, the substitution is meant to occur at any valency-allowed position on the system.

The term ‘diheteronucleophile’ as used herein refers to a compound having nucleophilic character at a heteroatom, usually nitrogen, that is directly bonded to another heteroatom. Typical examples include amine compounds having a nitrogen that is attached via a single bond to another heteroatom, typically selected from N, O and S. Common examples are

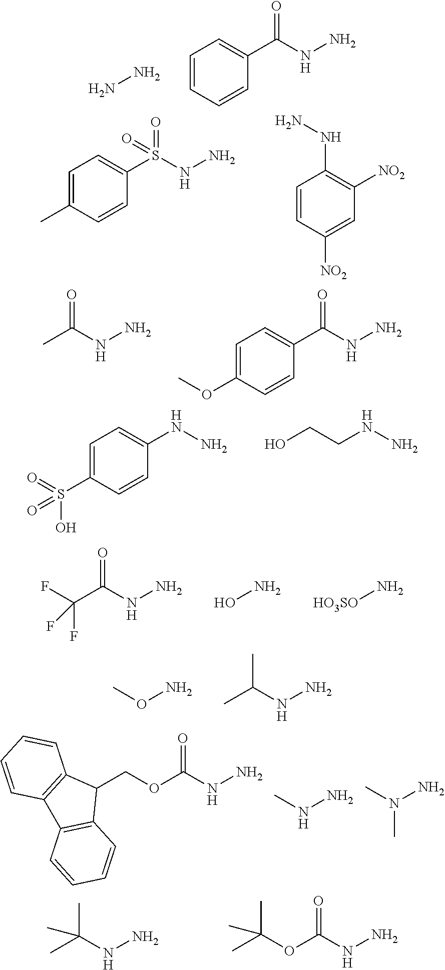

hydrazine and hydroxylamine compounds. The amine nitrogen may be substituted provided it retains nucleophilic character, and the attached N, O or S may also be substituted.

Structures of diheteronucleophiles described herein may be capable of forming multiple tautomers, as is well understood in the art. The particular tautomer or tautomers present often depend on solvent, pH, and other environmental factors as well as the structure itself. An example of tautomerism is shown here, where at least three different tautomers could be drawn to represent one compound:

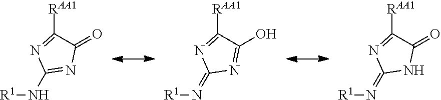

Where a compound can exist in more than one tautomeric form, typically one tautomer is depicted or described, and the structure is understood to represent each stable tautomer as well as mixtures of the tautomers. In particular, guanidine groups and heteroaryl groups substituted by hydroxyl or amine groups are often able to exist in multiple tautomers, and the description or depiction of one tautomer is understood to include the other tautomers of the same compound.

Methods described herein utilize ways to functionalize an N-terminal amino acid to form compounds of Formula (II), and to induce elimination of the functionalized NTAA of these compounds under mild conditions at around pH 5-10, as shown in Scheme I. More detailed description of these methods is provided in the published patent application WO 2020/223133 A1, and in U.S. provisional application Ser. No. 17/606,759.

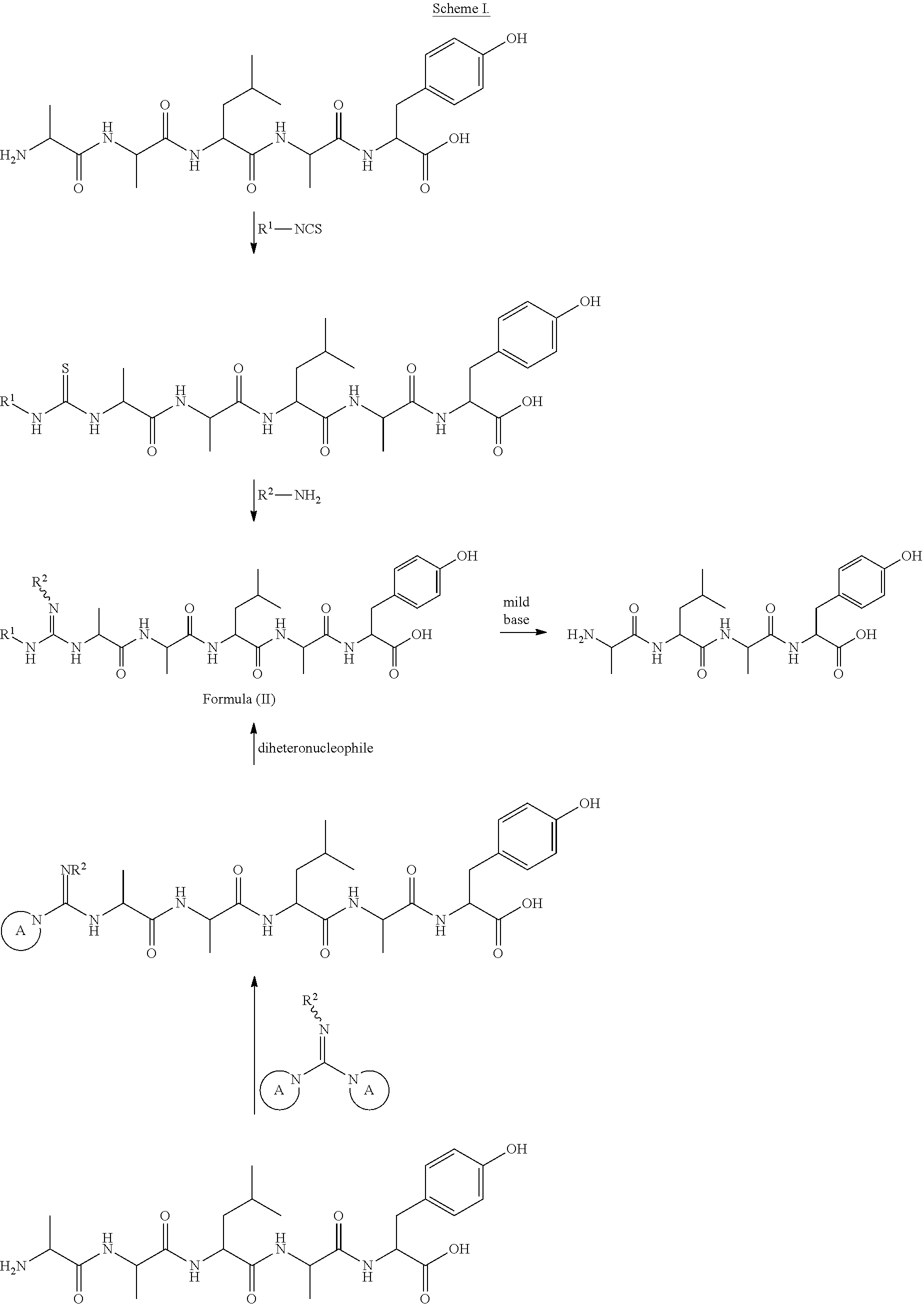

These reactions, as shown in Scheme I, result in cleavage of the NTAA from a polypeptide under mild conditions, and thus enable a method for removal of the NTAA from a polypeptide. Like Edman degradation, the cleavage of each NTAA produces a by-product that is determined by and therefore indicative of the structure of the NTAA that was removed. The described method can be used repeatedly, to remove one NTAA at a time from a polypeptide that contains coding tags attached to the specific amino acid residues.

The mild reaction conditions involved make it possible to perform these reactions in the presence of acid-sensitive moieties, such as nucleic acids. As a result, the methods can be combined with technology that utilizes nucleic acid tags to record information about each NTAA that is functionalized and removed, as the reactions are occurring. The nucleic acids are stable to the conditions used for functionalization and cleavage of the NTAA of a polypeptide as shown by data presented in the published patent application WO 2020/223133 A1, and in U.S. provisional application Ser. No. 17/606,759.

In some embodiments, functionalization of the NTAA using a chemical reagent comprising a compound of Formula (AA) and the subsequent elimination are as depicted in the following scheme:

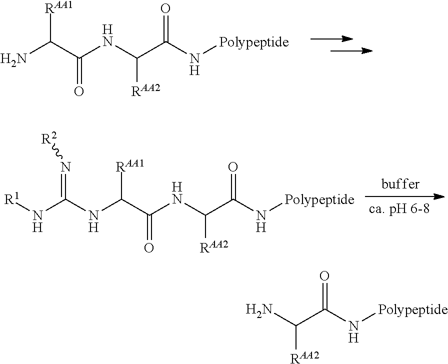

wherein R¹ and R² are as defined above and R^{AA1} is the side chain of the NTAA of a polypeptide.

In some embodiments, the polypeptide is obtained by fragmenting a protein from a biological sample. Examples of biological samples include, but are not limited to cells (both primary cells and cultured cell lines), cell lysates or extracts, cell organelles or vesicles, including exosomes, tissues and tissue extracts; biopsy; fecal matter; bodily fluids (such as blood, whole blood, serum, plasma, urine, lymph, bile, cerebrospinal fluid, interstitial fluid, aqueous or vitreous humor, colostrum, sputum, amniotic fluid, saliva, anal and vaginal secretions, perspiration and semen, a transudate, an exudate; microbial samples; research samples including extracellular fluids, extracellular supernatants from cell cultures, inclusion bodies in bacteria, cellular compartments including mitochondrial compartments, and cellular periplasm. A peptide, polypeptide, protein, or protein complex may comprise a standard, naturally occurring amino acid, a modified amino acid (e.g., post-translational modification), an amino acid analog, an amino acid mimetic, or any combination thereof.

In any of the embodiments provided herein, the functionalized NTAA is removed by a suitable reagent. Typically the formulation for NTAA removal is 1-100 mM of suitable reagent for NTAA removal in a non-nucleophilic medium at a pH of about 5-10. The medium typically comprises a buffering agent such as sodium/potassium phosphate, PBS, acetate, carbonate, bicarbonate, tertiary amine salts (e.g., N-ethylmorpholinium acetate, triethylammonium acetate, HEPES, MOPS, MES, POPSO, CAPSO, other Good's buffers), chloride, or Tris. The medium is typically aqueous and optionally comprises 0-80% of a water-miscible organic solvent , such as dimethylsulfoxide, N,N-dimethylformamide, N,N-dimethylacetamide, methanol, N-methylpyrrolidone, ethanol, or acetonitrile or a combination of two or more of these. The mixture is typically maintained at 25° C.-100° C. for 10-60 minutes in the medium to effect removal of the NTAA. An example of a suitable medium is water with phosphate, sodium chloride, tween 20 (surfactant) at pH 5-10, and is heated at 25° C.-60° C. for 1 to 60 minutes containing a suitable reagent such as a diheteronucleophile. In some embodiments, the elimination is performed using an aqueous formulation that includes 0.1M to 2.0M sodium, potassium, cesium, or ammonium phosphate buffer or sodium, potassium, or ammonium carbonate buffer at a pH 5.5-9.5 at 50-100° C. for 5-60 minutes. In some embodiments, the suitable reagent for NTAA elimination comprises a hydroxide, ammonia, or a diheteronucleophile, typically at a concentration of 0.15M-4.5M.

In some embodiments, the functionalized NTAA is eliminated using ammonia or ammonium hydroxide. In some embodiments, elimination of the functionalized NTAA is induced by treatment with a diheteronucleophile such as hydrazine or one of the hydrazine derivatives described herein. In some embodiments, the functionalized NTAA can be eliminated using a buffered solution without an amine, typically a mildly acidic or mildly basic (pH 5-9) medium, and in other embodiments ammonia, or a diheteronucleophilic amine such as one selected from this group A is present in the medium.

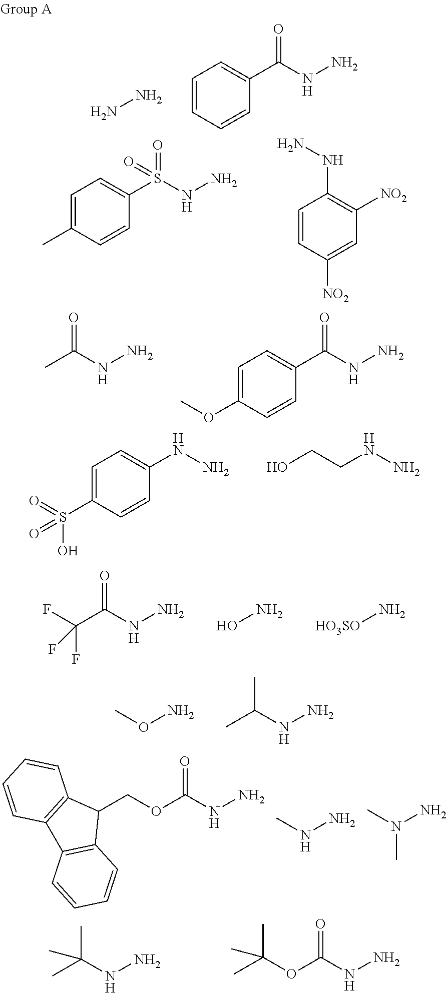

In a preferred embodiment (NTH), the diheteronucleophilic reagent is hydrazine.

In some embodiments, the polypeptide may be treated with one or more enzymes to eliminate the NTAA. In some examples, the polypeptide may be treated with an enzyme to eliminate the functionalized NTAA. In some cases, the polypeptide is treated with one or more enzymes before, during, or after the process of modifying the NTAA. The methods of the invention may include an optional step of treating a polypeptide with an enzyme to remove one or more NTAAs before, during, or after treatment with any of the provided chemical reagents; and kits for practicing methods of the invention may optionally include an enzyme to remove one or more NTAAs for use in this fashion. In some of any such embodiments, the polypeptide may be treated with a combination of enzymes to remove one or more NTAAs. In some embodiments, functionalized NTAAs of various polypeptides in a sample is eliminated via chemical and/or biological (e.g., enzymatic) means to expose a new NTAA.

In some specific examples, the polypeptide is treated with a proline aminopeptidase, a proline iminopeptidase (PIP), a pyroglutamate aminopeptidase (pGAP), an asparagine amidohydrolase, a peptidoglutaminase asparaginase, and/or a protein glutaminase, or a homolog thereof. This may be done before applying a chemical NTAA elimination step as described herein. In some embodiments, an enzyme treatment is compatible with the treatment with the provided chemical reagents and/or with steps performed in the polypeptide analysis assay. See e.g., Ito et al., 2012, Appl Environ Microbiol. 78(15): 5182-5188; Yamaguchi et al., 2001, Eur J Biochem. 268(5):1410-21.

A polypeptide may comprise L-amino acids, D-amino acids, or both. A polypeptide may comprise a standard, naturally occurring amino acid, a modified amino acid (e.g., post-translational modification), an amino acid analog, an amino acid mimetic, or any combination thereof. In some embodiments, the polypeptide is naturally occurring, synthetically produced, or recombinantly expressed. In any of the aforementioned embodiments, the polypeptide may further comprise a post-translational modification.

In some embodiments, after transferring identifying information of the barcode region or the region complementary to the barcode region from complementary coding tag covalently coupled to the NTAA of the polypeptide to the recording tag, the NTAA of the polypeptide is removed by an engineered enzyme.

In some embodiments, an engineered cleavase can remove or can be configured to remove any suitable single N-terminally modified amino acid from a target polypeptide. For example, the engineered cleavase can remove or can be configured to remove a N-terminal amino acid that is labeled with a chemical or an enzymatic reagent or moiety. In some embodiments, the engineered cleavase comprises a dipeptidyl aminopeptidase comprising at least two mutations in a substrate binding site, wherein the engineered cleavase removes or is configured to remove a single labeled terminal amino acid from a polypeptide. In some embodiments, the engineered cleavase is configured to cleave a peptide bond between a terminal labeled amino acid residue and a penultimate terminal amino acid residue of the polypeptide. In some embodiments, the engineered cleavase is derived from a dipeptidyl peptidase 3, dipeptidyl peptidase 5, dipeptidyl peptidase 7, dipeptidyl peptidase 11, dipeptidyl aminopeptidase BII, or from a protein classified in EC 3.4.14, EC 3.4.15, MEROPS S9, MEROPS S46, MEROPS M49. Some non-limited examples of the engineered cleavases configured to cleave the NTAA of the immobilized peptide are described in the published patent applications US 2021/0214701 A1 and WO 2021/141924 A1, incorporated herein.

In a preferred aspect of the invention, engineered cleavase enzymes used in the disclosed methods are made by recombinant techniques. A number of cloned polymerase genes are available or may be obtained using standard recombinant techniques, such as disclosed in Sambrook et al., In: Molecular Cloning A Laboratory Manual (2d ed.) Cold Spring Harbor Laboratory Press, Cold Spring Harbor, N.Y. (1989).

In some embodiments, engineered cleavase enzymes can be identified using a genetic screen. In some cases, the genetic screen uses a cell-based system. In some embodiments, the genetic screen uses prokaryotic cells, such as E. coli strains including E. coli variants or mutants. In some embodiments, the genetic selection is designed to select for modified cleavases with desired characteristics for binding of substrates, cleaving, and/or removal of labeled N-terminal amino acids, as disclosed in the published patent applications US 2021/0214701 A1 and WO 2021/141924 A1.

In some embodiments, carrying out a genetic selection screen involves preparing various cleavase genes (e.g., a dipeptidyl peptidase, a dipeptidyl aminopeptidase, a peptidyl-dipeptidase, a dipeptidyl carboxypeptidase, a sedolisin, or a tripeptidyl peptidase) for expression. A plasmid or cosmid containing nucleic acid sequences encoding mutated or modified cleavase polypeptides is readily constructed using standard techniques well known in the art. In some embodiments, the expression of any of the cleavases may further include a signal sequence. In some cases, the use of a signal sequence may be useful for purification purposes. For example, a periplasm targeting sequence such as PelB can be included in the expression construct. Recombinant vectors can be generated using any of the recombinant techniques known in the art.

In some embodiments, a kit for identifying a polypeptide immobilized on a solid support is provided, the kit comprising:

(a) a plurality of coding tags, wherein each coding tag of the plurality of coding tags is configured to react selectively with a specific type of amino acid residues from the polypeptide and comprises a barcode region with identifying information regarding the specific type of amino acid residue(s) to which the coding tag reacts selectively;

(b) a plurality of complementary coding tags, wherein each complementary coding tag of the plurality of complementary coding tags comprises (i) a region complementary to the barcode region of a corresponding coding tag, (ii) a first spacer region complementary to a first complementary spacer region of a recording tag associated with the polypeptide, and (iii) a moiety configured, when in a close proximity, to be covalently coupled to the NTAA of the polypeptide, or to a modified NTAA of the polypeptide.

In some embodiments, the specific type of amino acid residues is selected from the group consisting of: lysine, arginine, aspartate, glutamate, histidine, cysteine, serine, methionine, tryptophan and tyrosine.

In some embodiments, the moiety comprises a click chemistry reactive group.

In some embodiments, the moiety comprises an azide and is configured to be covalently coupled to the NTAA of the polypeptide modified with a propargyl halide or an activated propargylic acid. In other embodiments, the moiety comprises a propargyl halide or an activated propargylic acid and is configured to be covalently coupled to the NTAA of the polypeptide modified with an azide.

In some embodiments, the kit further comprises the solid support, wherein the recording tag is configured to be joined to the solid support.

In preferred embodiments, the solid support comprises a plurality of DNA hairpins immobilized on the solid support and configured to capture via hybridization the one or more nucleic acid recording tags associated with the polypeptide.

In some embodiments, the kit further comprises the recording tag configured to be associated with the polypeptide immobilized on the solid support.

In some embodiments, the kit further comprises an engineered enzyme or a modifying reagent configured to remove the NTAA of the polypeptide.

In some embodiments, the kit further comprises instructions for using the reagents of the kit for identifying a polypeptide immobilized on a solid support.

Another binder-free encoding (BFE) method provided herein is a method for identifying a polypeptide, the method comprising the steps of: (a) providing the polypeptide and an associated recording tag joined to a solid support; (b) contacting the polypeptide with a plurality of coding tags, wherein each coding tag of the plurality of coding tags is configured to react selectively with a specific type of amino acid residue(s) and comprises: i) a barcode region with identifying information regarding the specific type of amino acid residue(s) to which the coding tag reacts selectively, ii) an identification region unique for each specific type of amino acid residues to which the coding tags react selectively, and iii) a recognition region for a site-specific restriction enzyme, located between the barcode region and the identification region, thereby obtaining the polypeptide comprising coding tags attached to the specific amino acid residues; (c) providing conditions for hybridization of the recording tag to one of the coding tags attached to the specific amino acid residues, thereby forming a double stranded region, and extending the recording tag (using the coding tag as a template), thereby transferring information of the barcode region and the recognition region from the coding tag to the recording tag; (d) cutting the recognition region by providing the site-specific restriction enzyme, so that the extended recording tag is released and only a coding tag stub comprising the identification region of the coding tag remains attached to the polypeptide; (e) repeating steps (c) and (d) for all other coding tags attached to the specific amino acid residues of the polypeptide, thereby obtaining the polypeptide comprising coding tag stubs attached to the specific amino acid residues; (f) removing the NTAA of the polypeptide, thereby exposing a new NTAA; (g) adding a second order complementary spacer region to the recording tag extended at step (f); (h) restoring coding tags from the coding tag stubs using the identification region; (j) repeating steps (c)-(h) one or more times; and (k) analyzing the recording tag extended at step (j) by a nucleic acid sequencing method, and obtaining information regarding the specific amino acid residues of the polypeptide before and after removing step, thereby identifying the polypeptide.

The following embodiment illustrates an alternative exemplary workflow, which comprises an binder-free encoding (BFE) reaction, which does not use complementary coding tags having a moiety configured to be covalently coupled to the NTAA of the polypeptide, or to a modified NTAA of the polypeptide. Similar to the first described workflow, during sample preparation, the polypeptide comprising coding tags (CTs) attached to the specific amino acid residues is obtained. This approach enables sequencing of proteins without the need for sole identification of N-terminal amino acids (NTAAs) on peptides or costly methods required for developing protein binders. Both this and the first described workflow are useful for highly parallel, high throughput polypeptide identification.

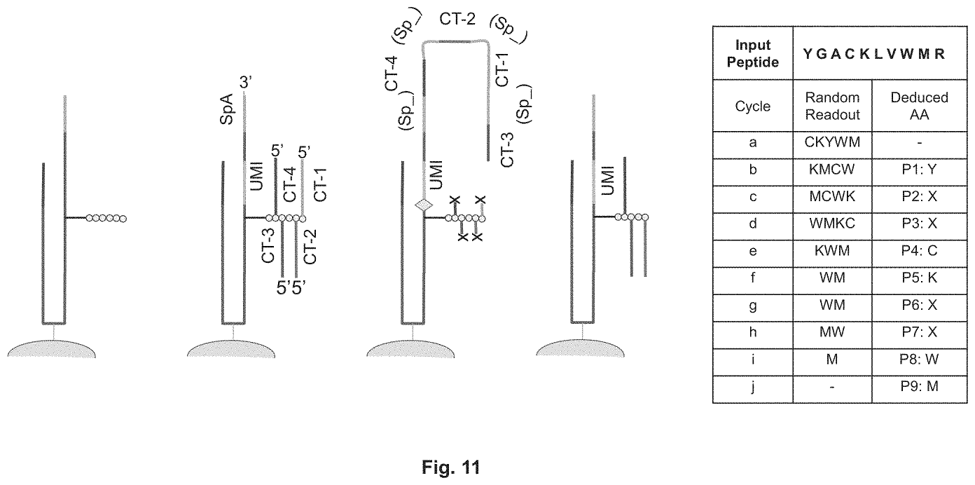

In this workflow, the polypeptide and an associated recording tag (polypeptide-recording tag (RT) conjugate) is immobilized onto a solid support (bead) containing capture DNAs anchored to the bead (FIG. 11). This anchored DNA strands are a partially double stranded hairpin with a site for hybridization and ligation of the polypeptide-RT conjugate. The RT is designed to receive information transfer from the CT barcode during the encoding process. Optionally, in order to prevent excessively long RTs which could hinder assay performance, each cycle includes the cleavage or nicking and denaturing, collection and restoration of every RT. There is a Unique Molecular Identifier (UMI) present on every RT which is cleaved or nicked and collected for accurate analysis. A UMI sequence is also present on every hairpin capture DNA. After the polypeptide-RT conjugate is captured on hairpin capture DNA and ligated, primer extension reaction copies the UMI onto the RT. This allows the exact same unique UMI to be copied to the RTs of different cycles for the same polypeptide-RT conjugate. After the UMI is copied onto the RT, a splint ligation adds a hybridizing spacer sequence to the RT complementary to a corresponding spacer sequence present in complementary coding tag, which will be used for transferring CT information to the RT on the next encoding cycle.

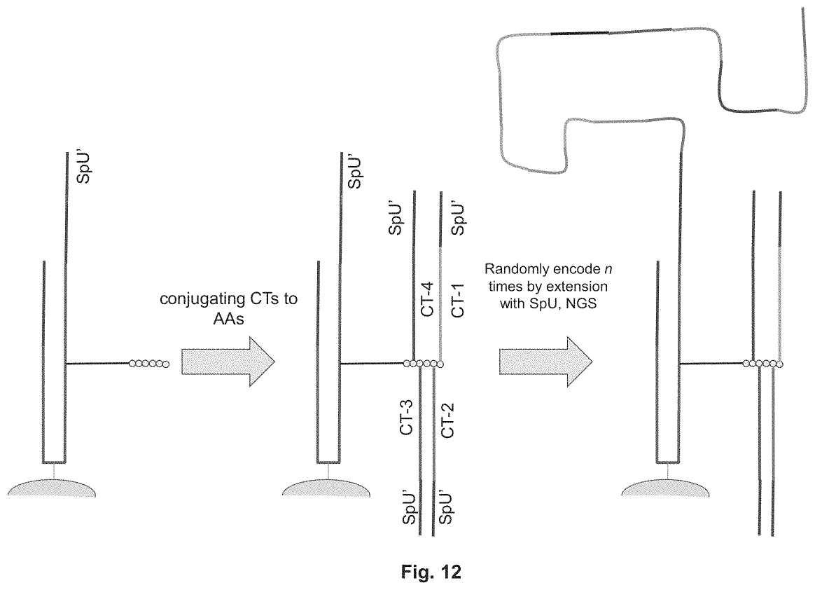

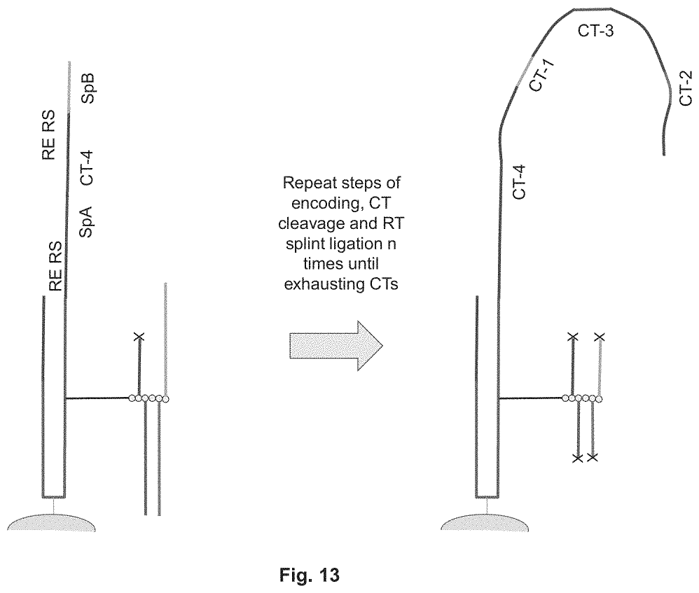

After the polypeptide-RT conjugates are immobilized on beads and the RT has a UMI and spacer, sequential steps of conjugating CTs to other reactive amino acid side chains are performed (FIG. 11). CTs encode identifiers of specific reactive amino acid residues in the form of barcodes. Not all amino acids need to be conjugated for polypeptide identification. Random sequential information transfer of all the CTs on a polypeptide is transferred during one encoding cycle to a RT (FIG. 11 and FIG. 12). After each CT to RT transfer, the CT is made unable to further hybridize with the RT so it will not reread the same CT in one cycle. The transfer is performed by splint ligation or extension followed by dsDNA restriction digestion (creating CT stubs) or nicking and denaturing (FIG. 11). There are enough nucleotide bases remaining on each CT for later restoration via splint ligation or extension in order to repeat further cycles of encoding (FIG. 13).

The NTAA and any conjugated partial CTs are cleaved by chemical or enzymatic methods and washed away after one complete cycle of encoding and exhausting all CTs (FIG. 11). The RT containing the CTs from one cycle and a UMI is cleaved or nicked and collected for later analysis. Splint ligations restoring the RT and CTs prepare the system for another cycle of encoding. The assay comprises multiple cycles of random CT transfers and cleavage or nicking, NTAA cleavage, RT cleavage or nicking then restoration, and CT restoration (FIG. 11 and FIG. 13).

A significantly simplified but lower resolution method of BFE could also be performed. Eliminating the need for any restriction enzymes, nicking endonucleases or peptide degradation would reduce the number of reagents and steps for the process. Utilizing a universal spacer on each CT would allow hybridization and extension on the RT. Simply denaturing the dsDNA and repeating this process n times would provide insight into the identity of the amino acids present on a peptide and the overall peptide as well through bioinformatic analysis.

The RT samples from each cycle of BFE are analyzed by NGS. By comparing the presence of the identifiable amino acids from one cycle to the next, it is possible to deduce the correct order, spacing and quantity of identifiable amino acids. This analysis can lead to the identification of peptides and ultimately proteins in a sample or a biological sample.

### EXEMPLARY EMBODIMENTS

Among the provided embodiments are:

1. A method for identifying a polypeptide, the method comprising the steps of:

(a) providing the polypeptide and an associated recording tag joined to a solid support;

(b) contacting the polypeptide with a plurality of coding tags, wherein each coding tag of the plurality of coding tags is configured to react selectively with a specific type of amino acid residue(s) and comprises a barcode region with identifying information regarding the specific type of amino acid residue(s) to which the coding tag reacts selectively, thereby obtaining the polypeptide comprising coding tags attached to the specific amino acid residues;

(c) contacting the polypeptide comprising coding tags attached to the specific amino acid residues with a plurality of complementary coding tags, wherein each complementary coding tag of the plurality of complementary coding tags comprises (i) a region complementary to the barcode region of a corresponding coding tag, (ii) a first spacer region complementary to a first complementary spacer region of the recording tag, and (iii) a moiety configured, when in a close proximity, to be covalently coupled to the NTAA of the polypeptide, or to a modified NTAA of the polypeptide;

(d) providing conditions for covalently coupling the moiety to the NTAA of the polypeptide or the modified NTAA of the polypeptide;

(e) removing complementary coding tags that are not covalently coupled to the NTAA of the polypeptide;

(f) transferring identifying information of the barcode region or the region complementary to the barcode region from complementary coding tag covalently coupled to the NTAA of the polypeptide to the recording tag, wherein transferring the identifying information comprises a primer extension or ligation;

(g) removing the NTAA of the polypeptide, thereby exposing a new NTAA;

(h) adding a second order complementary spacer region to the recording tag extended at step (f);

(j) repeating steps (c)-(h) one or more times by replacing at step (c) the first spacer region of the complementary coding tags with a second or higher order spacer region complementary to the second or higher order complementary spacer region of the recording tag, and by replacing at step (h) the second complementary spacer region with a third or higher order complementary spacer region; and

(k) analyzing the recording tag extended at step (j) by a nucleic acid sequencing method, and obtaining information regarding the specific amino acid residues of the polypeptide, thereby identifying the polypeptide.

2. The method of embodiment 1, wherein at step (f) transferring information is performed by the primer extension using a DNA polymerase having a strand-displacement ability.

3. The method of embodiment 1, wherein at step (f) transferring information is performed by a splint ligation.

4. The method of any one of embodiments 1-3, wherein the NTAA of the polypeptide is removed by an engineered enzyme.

5. The method of any one of embodiments 1-4, wherein the NTAA of the polypeptide is removed by the following method:

(a) functionalizing the N-terminal amino acid (NTAA) of the polypeptide with a chemical reagent, wherein the chemical reagent is either:

(i) a compound of Formula (AA):

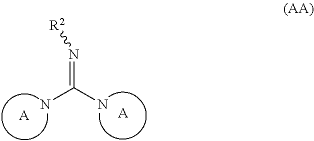

wherein: R2 is H or R4;

R4 is C1-6 alkyl, which is optionally substituted with one or two members selected from halo, C1-3 alkyl, C1-3 alkoxy, C1-3 haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C1-3 alkyl, C1-3 alkoxy, C1-3 haloalkyl, NO2, CN, COOR″, and CON(R″)2,

where each R″ is independently H or C1-3 alkyl;

each ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C1-4 alkyl, C1-4 alkoxy, —OH, halo, C1-4 haloalkyl, NO2, COOR, CONR2, —SO2R*, —NR2, phenyl, and 5-6 membered heteroaryl;

wherein each R is independently selected from H and C1-3 alkyl optionally substituted with OH, OR*, —NH2, —NHR*, or —NR*2; and

each R* is C1-3 alkyl, optionally substituted with OH, oxo, C1-2 alkoxy, or CN;

wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C1-2 alkyl, OH, oxo, C1-2 alkoxy, or CN; or

(ii) a compound of the formula R3-NCS;

wherein R3 is H or an optionally substituted group selected from phenyl, 5-membered heteroaryl, 6-membered heteroaryl, C1-3 haloalkyl, and C1-6 alkyl,

wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;

where each R′ is independently H or C₁₋₃ alkyl;

wherein two R′ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;

to provide an initial NTAA functionalized polypeptide;

optionally treating the initial NTAA functionalized polypeptide with an amine of Formula R²—NH₂ or with a diheteronucleophile to form a secondary NTAA functionalized polypeptide; and

(b) treating the initial NTAA functionalized polypeptide or the secondary NTAA functionalized polypeptide with a suitable medium to eliminate the NTAA, thereby removing the NTAA of the polypeptide.

6. The method of embodiment 5, wherein the suitable medium has pH between about 5 and about 9, and optionally includes a hydroxide, carbonate, phosphate, sulfate or amine.

7. The method embodiment 5 or embodiment 6, wherein treating the initial NTAA functionalized polypeptide or the secondary NTAA functionalized polypeptide with the suitable medium occurs at temperature between about 40° C. and about 95° C.

8. The method of any one of embodiments 1-7, wherein the moiety comprises a click chemistry reactive group.

9. The method of any one of embodiments 1-8, wherein either the moiety comprises an azide and is configured to be covalently coupled to the NTAA of the polypeptide modified with a propargyl halide or an activated propargylic acid, or the moiety comprises a propargyl halide or an activated propargylic acid and is configured to be covalently coupled to the NTAA of the polypeptide modified with an azide.

10. The method of any one of embodiments 1-9, wherein the moiety comprises a photocleavable linker.

11. The method of any one of embodiments 1-10, which is conducted to identify a plurality of polypeptides.

12. The method of any one of embodiments 1-11, wherein step (k) comprises bioinformatically matching the obtained information regarding the specific amino acid residues of the polypeptide with corresponding information extracted from a genomic or proteomic database.

13. The method of any one of embodiments 1-12, wherein at step (a) the polypeptide is covalently attached to the associated recording tag.

14. The method of any one of embodiments 1-13, wherein the specific type of amino acid residues is selected from the group consisting of: lysine, arginine, aspartate, glutamate, histidine, cysteine, serine, methionine, tryptophan and tyrosine.

15. The method of any one of embodiments 1-14, wherein before contacting the polypeptide with the plurality of coding tags, the polypeptide is at least partially denatured to expose the specific amino acid residues to the plurality of coding tags.

16. The method of any one of embodiments 1-15, wherein a plurality of polypeptides are provided at step (a) on the same solid support, wherein each polypeptide is associated with a separate recording tag.

17. A kit for identifying a polypeptide immobilized on a solid support, comprising:

(a) a plurality of coding tags, wherein each coding tag of the plurality of coding tags is configured to react selectively with a specific type of amino acid residue(s) from the polypeptide and comprises a barcode region with identifying information regarding the specific type of amino acid residue(s) to which the coding tag reacts selectively; and

(b) a plurality of complementary coding tags, wherein each complementary coding tag of the plurality of complementary coding tags comprises (i) a region complementary to the barcode region of a corresponding coding tag, (ii) a first spacer region complementary to a first complementary spacer region of a recording tag associated with the polypeptide, and (iii) a moiety configured, when in a close proximity, to be covalently coupled to the NTAA of the polypeptide, or to a modified NTAA of the polypeptide.

18. The kit of embodiment 17, wherein the specific type of amino acid residues is selected from the group consisting of: lysine, arginine, aspartate, glutamate, histidine, cysteine, serine, methionine, tryptophan and tyrosine.

19. The kit of embodiment 17 or embodiment 18, wherein the moiety comprises a click chemistry reactive group.

20. The kit of any one of embodiments 17-19, wherein either the moiety comprises an azide and is configured to be covalently coupled to the NTAA of the polypeptide modified with a propargyl halide or an activated propargylic acid, or the moiety comprises a propargyl halide or an activated propargylic acid and is configured to be covalently coupled to the NTAA of the polypeptide modified with an azide.

21. The kit of any one of embodiments 17-20, further comprising the solid support, wherein the recording tag is configured to be joined to the solid support.
In preferred embodiments, the solid support comprises a plurality of DNA hairpins immobilized on the solid support and configured to capture via hybridization the one or more nucleic acid recording tags associated with the polypeptide.

22. The kit of any one of embodiments 17-21, further comprising the recording tag configured to be associated with the polypeptide immobilized on the solid support.

23. The kit of any one of embodiments 17-22, further comprising an engineered enzyme or a modifying reagent configured to remove the NTAA of the polypeptide.

24. A method for identifying a polypeptide, the method comprising the steps of:

(a) providing the polypeptide and an associated recording tag joined to a solid support;

(b) contacting the polypeptide with a plurality of coding tags, wherein each coding tag of the plurality of coding tags is configured to react selectively with a specific type of amino acid residue(s) and comprises: i) a barcode region with identifying information regarding the specific type of amino acid residue(s) to which the coding tag reacts selectively, ii) an identification region unique for each specific type of amino acid residue(s) to which the coding tags react selectively, and iii) a recognition region for a site-specific restriction enzyme, located between the barcode region and the identification region, thereby obtaining the polypeptide comprising coding tags attached to the specific amino acid residues;

(c) providing conditions for hybridization of the recording tag to one of the coding tags attached to the specific amino acid residue(s), thereby forming a double stranded region, and extending the recording tag, thereby transferring information of the barcode region and the recognition region from the coding tag to the recording tag;

(d) cutting the recognition region by providing the site-specific restriction enzyme, so that the extended recording tag is released and only a coding tag stub comprising the identification region of the coding tag remains attached to the polypeptide;

(e) repeating steps (c) and (d) for all other coding tags attached to the specific amino acid residues of the polypeptide, thereby obtaining the polypeptide comprising coding tag stubs attached to the specific amino acid residues;

(f) removing the NTAA of the polypeptide, thereby exposing a new NTAA;

(g) adding a second order complementary spacer region to the recording tag extended at step (f);

(h) restoring coding tags from the coding tag stubs;

(j) repeating steps (c)-(h) one or more times; and

(k) analyzing the recording tag extended at step (j) by a nucleic acid sequencing method, and obtaining information regarding the specific amino acid residues of the polypeptide before and after removing step, thereby identifying the polypeptide.

25. The method of embodiment 24, wherein the NTAA of the polypeptide is removed by an engineered enzyme.

26. The method of embodiment 24 or embodiment 25, wherein the NTAA of the polypeptide is removed by the following method:

(a) functionalizing the N-terminal amino acid (NTAA) of the polypeptide with a chemical reagent, wherein the chemical reagent is either:

(i) a compound of Formula (AA):

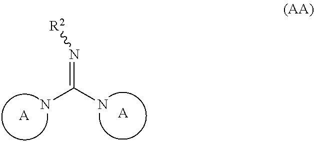

wherein: R2 is H or R4;

R4 is C1-6 alkyl, which is optionally substituted with one or two members selected from halo, C1-3 alkyl, C1-3 alkoxy, C1-3 haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C1-3 alkyl, C1-3 alkoxy, C1-3 haloalkyl, NO2, CN, COOR″, and CON(R″)2,

where each R″ is independently H or C1-3 alkyl;

each ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C1-4 alkyl, C1-4 alkoxy, —OH, halo, C1-4 haloalkyl, NO2, COOR, CONR2, —SO2R*, —NR2, phenyl, and 5-6 membered heteroaryl;

wherein each R is independently selected from H and C1-3 alkyl optionally substituted with OH, OR*, —NH2, -NHR*, or —NR*2; and

each R* is C1-3 alkyl, optionally substituted with OH, oxo, C1-2 alkoxy, or CN;

wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C1-2 alkyl, OH, oxo, C1-2 alkoxy, or CN; or

(ii) a compound of the formula R3-NCS;

wherein R3 is H or an optionally substituted group selected from phenyl, 5-membered heteroaryl, 6-membered heteroaryl, C1-3 haloalkyl, and C1-6 alkyl,

wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;

where each R′ is independently H or C₁₋₃ alkyl;

wherein two R′ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;

to provide an initial NTAA functionalized polypeptide;

optionally treating the initial NTAA functionalized polypeptide with an amine of Formula R²—NH₂ or with a diheteronucleophile to form a secondary NTAA functionalized polypeptide; and

(b) treating the initial NTAA functionalized polypeptide or the secondary NTAA functionalized polypeptide with a suitable medium to eliminate the NTAA, thereby removing the NTAA of the polypeptide.

27. The method of embodiment 26, wherein the suitable medium has pH between about 5 and about 9, and optionally includes a hydroxide, carbonate, phosphate, sulfate or amine.

28. The method of any one of embodiments 24-27, wherein treating the initial NTAA functionalized polypeptide or the secondary NTAA functionalized polypeptide with the suitable medium occurs at temperature between about 40° C. and about 95° C.

29. The method of any one of embodiments 24-28, wherein at step (a) the polypeptide is covalently attached to the associated recording tag.

30. The method of any one of embodiments 24-29, wherein the specific type of amino acid residues is selected from the group consisting of: lysine, arginine, aspartate, glutamate, histidine, cysteine, serine, methionine, tryptophan and tyrosine.

### EXAMPLES

The following examples are offered to illustrate but not to limit the methods, compositions, and uses provided herein. Certain aspects of the present invention, including, but not limited to, embodiments for the Proteocode™ polypeptide sequencing assay, information transfer between coding tags and recording tags, methods of making nucleotide-polypeptide conjugates, methods for attachment of nucleotide-polypeptide conjugates to a support, methods of generating barcodes, methods of generating specific binders recognizing an N-terminal amino acid of a polypeptide, reagents and methods for modifying and/or removing an N-terminal amino acid from a polypeptide, methods for analyzing extended recording tags were disclosed in the following published patent applications: US 2019/0145982 A1, US 2020/0348308 A1, US 2020/0348307 A1, US 2021/0208150 A1, US 11427814 B2, US 2022/0049246 A1, the contents of which are incorporated herein by reference in their entireties.

### Example 1. Preparation of Protein Lysates

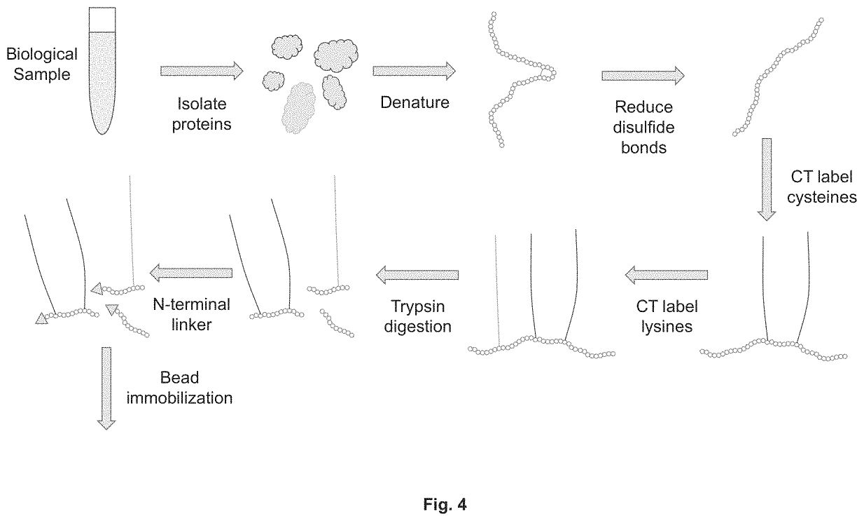

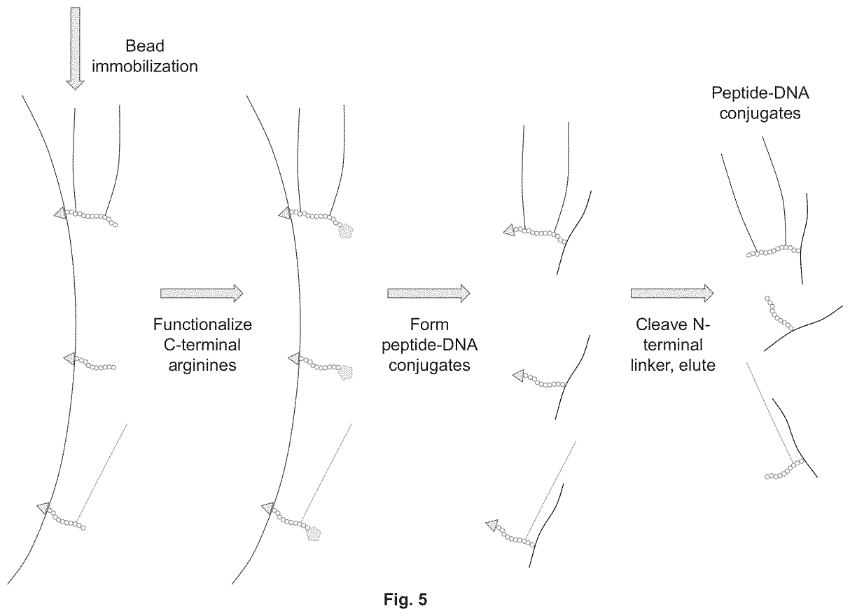

In this example, polypeptides used in the disclosed binder-free encoding methods are obtained by processing a biological sample, such as a cell lysate. There are a wide variety of protocols known in the art for making protein lysates from various sample types. Most variations on the protocol depend on cell type and whether the extracted proteins in the lysate are to be analyzed in a non-denatured or denatured state. For the disclosed binder-free encoding methods, either native conformation or denatured proteins can be immobilized to a solid support (see e.g., FIG. 4 and FIG. 5). Moreover, after immobilization of native proteins, the proteins immobilized on the solid support can be denatured. The advantage of employing denatured proteins is to improve accessibility of specific amino acid residues to which the coding tags react selectively. Additionally, the method workflow is simplified when using denatured proteins since the annealed complementary coding tags that are not covalently coupled to the NTAA of the polypeptide, as well as the annealed complementary coding tag can be stripped from the extended recording tag using alkaline (e.g., 0.1 NaOH) stripping conditions since the immobilized protein is already denatured. This contrasts with the removal of annealed complementary coding tags using assays comprising proteins in their native conformation, that require an enzymatic removal of the annealed complementary coding tag following binding event and information transfer.

Examples of non-denaturing protein lysis buffers include: RPPA buffer consisting of 50 mm HEPES (pH 7.4), 150 mM NaCl, 1% Triton X-100, 1.5 mM MgCl2, 10% glycerol; and commercial buffers such as M-PER mammalian protein extraction reagent (Thermo-Fisher). An exemplary denaturing lysis buffer comprises 50 mm HEPES (pH 8), 1% SDS. The addition of Urea (1M-3M) or Guanidine HCl (1-8M) can also be used in denaturing the protein sample. In addition to the above components of lysis buffers, protease and phosphatase inhibitors are also generally included. Examples of protease inhibitors and typical concentrations include aptrotinin (2 μg/ml), leupeptin (5-10 μg/ml), benzamidine (15 μg/ml), pepstatin A (1 μg/ml), PMSF (1 mM), EDTA (5 mM), and EGTA (1 mM). Examples of phosphatase inhibitors include Na pyrophosphate (10 mM), sodium fluoride (5-100 mM) and sodium orthovanadate (1 mM). Additional additives can include DNAaseI to remove DNA from the protein sample, and reducing agents such as DTT to reduce disulfide bonds.

An example of a non-denaturing protein lysate protocol prepared from tissue culture cells is as follows: Adherent cells are trypsinized (0.05% trypsin-EDTA in PBS), collected by centrifugation (200 g for 5 min.), and washed 2× in ice cold PBS. Ice-cold M-PER mammalian extraction reagent (^{˜}1 mL per 107 cells/100 mm dish or 150 cm2 flask) supplemented with protease/phosphatase inhibitors and additives (e.g., EDTA free complete inhibitors (Roche) and PhosStop (Roche) is added. The resulting cell suspension is incubated on a rotating shaker at 4° C. for 20 min. and then centrifuged at 4° C. at 12,000 rpm (depending on cell type) for 20 min to isolate the protein supernatant. The protein is quantitated using the BCA assay, and resuspended at 1 mg/ml in PBS. The protein lysates can be used immediately or snap frozen in liquid nitrogen and stored at −80° C.

An example of a denaturing protein lysate protocol, based on the SP3 protocol of Hughs et al., prepared from tissue culture cells is as follows: adherent cells are trypsinized (0.05% trypsin-EDTA in PBS), collected by centrifugation (200 g for 5 min.), and washed 2× in ice cold PBS. Ice-cold denaturing lysis buffer (^{˜}1 mL per 107 cells/100 mm dish or 150 cm2 flask) supplemented with protease/phosphatase inhibitors and additives (e.g., 1× complete Protease Inhibitor Cocktail (Roche)) is added. The resulting cell suspension is incubated at 95° C. for 5 min and placed on ice for 5 min. Benzonase Nuclease (500 Uml) is added to the lysate and incubated at 37° C. for 30 min to remove DNA and RNA. The proteins are reduced by addition of 5 μL of 200 mM DTT per 100 uL of lysate and incubated for 45° C. for 30 min. Alklylation of protein cysteine groups is accomplished by addition of 10 uL of 400 mM iodoacetamide per 100 uL of lysate and incubated in the dark at 24° C. for 30 min. Reactions are quenched by addition of 10 uL of 200 mM DTT per 100 uL of lysate. Proteins are optionally acylated by adding 2 ul an acid anhydride and 100 ul of 1 M Na2CO3 (pH 8.5) per 100 ul of lysate. Incubate for 30 min at room temp. Valeric, benzoic, and proprionic anhydride are recommended rather than acetic anhydride to enable “in vivo” acetylated lysines to be distinguished from “in situ” blocking of lysine groups by acylation (Sidoli, Yuan et al. 2015). The reaction is quenched by addition of 5 mg of Tris(2-aminoethyl)amine, polymer (Sigma) and incubation at room temperature for 30 min. Polymer resin is removed by centrifuging lysate at 2000 g for 1 min through a 0.45 um cellulose acetate Spin-X tube (Corning). The protein is quantitated using the BCA assay, and resuspended at 1 mg/ml in PBS.

### Example 2. Immobilization of Recording Tag-Labeled Polypeptides to a Solid Support

Recording tag-labeled polypeptides are immobilized on a substrate via an IEDDA click chemistry reaction using an mTet group on the recording tag and a TCO group on the surface of activated beads (solid support). 200 ng of M-270 TCO beads are resuspended in 100 ul phosphate coupling buffer. 5 pmol of DNA recording tag labeled peptides comprising an mTet moiety on the recording tag is added to the beads for a final concentration of 50 nM. The reaction is incubated for 1 hr at room temperature. After immobilization, unreacted TCO groups on the substrate are quenched with 1 mM methyl tetrazine acid in phosphate coupling buffer for 1 hr at room temperature.

Magnetic beads suitable for click-chemistry immobilization are created by converting M-270 amine magnetic Dynabeads to either azide or TCO-derivatized beads capable of coupling to alkyne or methyl Tetrazine-labeled oligo-peptide conjugates, respectively (see also Examples 20-21 of US 20190145982 A1). Namely, 10 mg of M-270 beads are washed and resuspended in 500 ul borate buffer (100 mM sodium borate, pH 8.5). A mixture of TCO-PEG (12-120)-NHS (Nanocs) and methyl-PEG (12-120)-NHS is resuspended at 1 mM in DMSO and incubated with M-270 amine beads at room temperature overnight. The ratio of the Methyl to TCO PEG is titrated to adjust the final TCO surface density on the beads such that there is <100 TCO moieties/um2. Unreacted amine groups are capped with a mixture of 0.1M acetic anhydride and 0.1M DIEA in DMF (500 ul for 10 mg of beads) at room temperature for 2 hrs. After capping and washing 3× in DMF, the beads are resuspended in phosphate coupling buffer at 10 mg/ml.

### Example 3. Polypeptide Immobilization Using Nucleic Acid Hybridization and Joining to a Solid Support

This example describes exemplary methods for joining (immobilizing) nucleic acid-polypeptide conjugates, such as conjugates of a polypeptide with recording tag, to a solid support. In a hybridization based method of immobilization, nucleic acid-polypeptide conjugates were hybridized and ligated to hairpin capture DNAs that were chemically immobilized on magnetic beads. The capture nucleic acids were conjugated to the beads using trans-cyclooctene (TCO) and methyltetrazine (mTet)-based click chemistry. TCO-modified short hairpin capture nucleic acids (16 basepair stem, 5 base loop, 24 base 5′ overhang) were reacted with mTet-coated magnetic beads. Phosphorylated nucleic acid-polypeptide conjugates (10 nM) were annealed to the hairpin DNAs attached to beads in 5× SSC, 0.02% SDS, and incubated for 30 minutes at 37° C. The beads were washed once with PBST and resuspended in 1× Quick ligation solution (New England Biolabs, USA) with T4 DNA ligase. After a 30-minute incubation at 25° C., the beads were washed twice with PBST and resuspended in the 50 μL of PBST. The total immobilized nucleic acid-polypeptide conjugates including amino FA-terminal peptides (FAGVAMPGAEDDVVGSGSK; SEQ ID NO: 3), amino AFA-terminal peptides (AFAGVAMPGAEDDVVGSGSK; SEQ ID NO: 4), and an amino AA-terminal peptides (AAGVAMPGAEDDVVGSGSK; SEQ ID NO: 5) were quantified by qPCR using specific primer sets. For comparison, peptides were immobilized onto beads using a non-hybridization based method that did not involve a ligation step. The non-hybridization based method was performed by incubating 30 μM TCO-modified DNA-tagged peptides including amino FA-terminal peptides, amino AFA-terminal peptides, and amino AA-terminal peptides, with mTet-coated magnetic beads overnight at 25° C.

As shown in Table 1, similar Ct values were observed in the non-hybridization preparation method with 1:100,000 grafting density and the hybridization based preparation method with 1:10,000 grafting density. Loading amount of DNA-tagged peptides for the hybridization based preparation method was 1/3000 compared to that for the non-hybridization preparation method. In general, it was observed that less starting material was needed for the hybridization based immobilization method.

TABLE 1
Comparison of Loading Hybridization and Non-hybridization
Immobilization Methods

| | Non-hybridization based | Hybridization based |
|---|---|---|
| | immobilization method | immobilization method |
| Grafting:Passivation | (−Ligation) | (+Ligation) |
| 1:100,000 | 19.4 | 25.4 |
| 1:10,000 | — | 21.1 |

### Example 4. Assessment of Encoding Function of Analytes Prepared and Barcoded Using Various Methods

This example describes two exemplary methods for coupling (immobilizing) nucleic acid-polypeptide conjugates to a solid support and various methods for attaching barcodes, UMIs, or other nucleic acid tags or components to the recording tag or capture nucleic acid.

In the methods described in this example, hybridization-based immobilization of the peptide was performed substantially as described in Example 3, except that the beads were washed three times after ligation (PBST, NaOH, and PBST). More experimental details on immobilization approach can be found in U.S. patent publication US 2022/0049246 A1, incorporated herein by reference.

In Method 1, which uses a scheme generally depicted in FIG. 6, amino FA peptides (FAGVAMPGAEDDVVGSGSK; SEQ ID NO: 3) were attached to bait nucleic acid (/5Phos/CAAGTTCTCAGTAATGCGTAGCCGCGACACTAG; SEQ ID NO: 6). The bait nucleic acid-peptide conjugates were loaded onto beads which had capture nucleic acids attached. The capture nucleic acids on the beads included a barcode template (CACTCAGTCCATTAAC CTAGTGTCGCGGACTACGCATTACTGAGA AGCTTGCTAGTCGACGTGGTCCTTTTGGACCACGTCGACTAG; SEQ ID NO: 7) at the 5′ end of the capture nucleic acid. The barcode templates each contained one dU site. The bait nucleic acid-peptide conjugates were attached to the capture nucleic acids using hybridized-based immobilization and coupling. The barcoding was performed using extension on beads using the barcode template located at the 5′ end of the capture nucleic acids. 50 μL of barcoding mixture was used which included 1× Custom Buffer (New England BioLabs, USA), 0.125 mM dNTPs and 0.125 units/μL Klenow fragment (3′→5′ exo-) (MCLAB, USA) and the reaction was incubated at 37° C. for 5 minutes. After transferring the barcode onto the bait nucleic acids by extension, the beads were washed twice with PBST. The barcode template on the capture nucleic acids used for extension were digested by incubation at 37° C. for 30 minutes with 2.5 units of USER Enzyme (New England BioLabs, USA). In this method, a Hind III restriction site was formed if extension occurred on capture nucleic acids that did not have bait nucleic acid-peptide conjugates attached. A 50 μL restriction enzyme solution including 1× Custom Buffer and 2.5 Units of Hind III (New England BioLabs, USA) was added to the samples and incubated at 37° C. for 30 minutes to digest these capture nucleic acids that were barcoded but not attached with a bait nucleic acid-peptide conjugate. If a bait nucleic-acid peptide conjugate was attached to the capture nucleic acid and barcoding occurred by extension onto the bait nucleic acid, then a Hind III site is not formed. The resulting beads were washed once with PBST, once with 0.1 M NaOH and once with PBST.

In Method 2, amino FA peptides (FAGVAMPGAEDDVVGSGSK; SEQ ID NO: 3) were attached to bait nucleic acids (SEQ ID NO: 6). Splint DNAs which contained sequence that is complementary to a portion of the bait nucleic acid and a portion of the barcode were used to bridge the bait nucleic acids and barcodes via hybridization. The barcoding was performed in 50 μL of barcoding mixture including 1× Quick Ligase Buffer (New England BioLabs, USA), 1.5 μM of splint DNA (CCATTAACCTAGTGTCGC; SEQ ID NO: 12), 2 μM of barcode (/5Phos/GTTAATGGACTGAGTG; SEQ ID NO: 13), 1 μM bait nucleic acid-tagged peptide and 2.5 units Quick Ligase (New England BioLabs, USA) at 25° C. for 5 minutes. After attaching the barcodes onto the bait nucleic acids of the bait nucleic acid-peptide conjugates, EDTA was added to the reaction at 50 mM to quench the ligase and the splint DNAs were washed away with NaOH. The resulting barcoded bait nucleic acid-peptide conjugates were diluted to 10 nM and attached to capture nucleic acids (e.g., SEQ ID NO: 7) using the hybridization-based method.

### Example 5. Use of Error Prone Library and SSM Library of S46 Dipeptidyl Peptidases to Evolve for a Modified Dipeptidyl Peptidase (Cleavase)

To engineer modified dipeptidyl peptidases that is configured to cleave the modified NTAA linked to a nucleic acid coding tag from the polypeptide, site saturation mutagenesis and error prone libraries of DAP BII dipeptidyl peptidases are created as disclosed in U.S. Pat. No. 11,427,814, incorporated herein by reference. The variant libraries are transformed into an arginine auxotroph strain of E. coli, which has a deletion in the argA gene (strain JW2786-1). Genetic selection is performed on the transformed E. coli using M9 minimal media agar plates supplemented with arginine N-terminal modified peptides. In these peptides, the N-terminal modification is comprised of a modifying moiety conjugating to the alpha amine of the N-terminus, and a tag moiety. Several NTM agents are screened including those that was used to label the N-terminal of arginine-containing peptides. The plates are incubated at 35° C. until colonies appear. From the surviving cells, plasmid DNA is subsequently isolated and sequenced to identify the mutations that generate an active modified dipeptidyl cleavase that recognizes labeled NTAAs and tolerates a longer nucleic acid tag at the substrate binding pockets. After sequence verification, these engineered dipeptidyl peptidases are expressed, purified and tested against the modified polypeptide substrates. After verification of these clones, novel mutations that are identified are combined to create new libraries to further improve the performance of modified dipeptidyl peptidases.

### Example 6. Exploring Starting Scaffolds of Other DPP and Aminopeptidase Families

S46 family of dipeptidyl peptidases has an extra capping domain that can potentially limit the catalytic activity on modified substrate having a nucleic acid tag. This limitation is mitigated by using couplers with a long linker (such as a PEG linker) for tethering of the coupler-polypeptide complex. Instead of a DAP BII scaffold, additional scaffolds will be used to engineer modified dipeptidyl peptidases that are configured to cleave the modified NTAA from the polypeptide, for example, DPPS and iminopeptidase. These families of enzymes have a different capping domain (different secondary structure and/or size), and are preferable to allow easier access of modified substrates having a nucleic acid tag based on structural modeling. A sequential two-step enzyme engineering approach will be employed similar to the approach for the S46 family disclosed in Example 5 and in U.S. Pat. No. 11,427,814, incorporated herein by reference. In the first step, the genetic selection platform to evolve native peptidases to modified peptidases will be used, and in the second step, mutations will be introduced around of the substrate binding site of the peptidases in areas that are involved in recognition of the tag moiety (“mod pocket”) and the terminal amino acid (“S2 pocket”) to create tolerance for tagged substrates.

### Example 7. Engineered Dipeptide Cleavases can Remove Single Labeled NTAAs of a Model Polypeptide

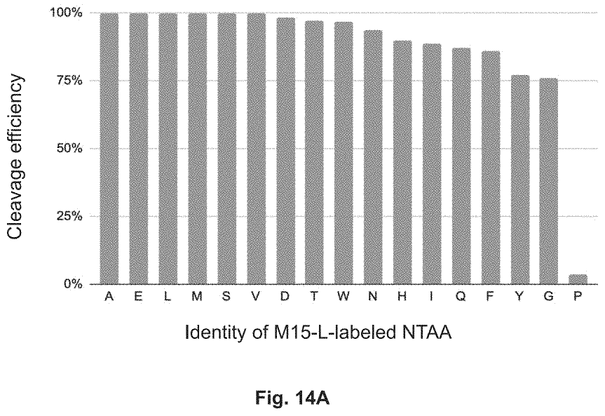

A set of dipeptide cleavase enzymes was evolved from an S46 DPP library as described in Example 5 to recognize and cleave a modified NTAA using M15-L-P1 target polypeptides (polypeptide sequences: M15-L-P1-Prest, where M15 is a 2-aminobenzamide, P1 is one of the 17 natural amino acid residues, excluding C, K, R), Prest is a dipeptide (such as AR) and the dipeptide cleavase scaffold from Thermomonas hydrothermalis (SEQ ID NO: 10). The enzymes can efficiently cleave M15-L-labeled polypeptides between P1 and P2 amino acid residues (the P2 residue is alanine), thus are configured to remove a single labeled terminal amino acid from the polypeptide (FIG. 14A and FIG. 14B). To accommodate the M15-L label in the substrate binding site, all modified dipeptide cleavases contained the following mutations at the conserved residues that form an amine binding site in unmodified dipeptidyl aminopeptidases: N214M, W215G, R219T, N329R, D673A (the indicated residue numbers correspond to positions of SEQ ID NO: 10). The cleavage efficiency of the evolved enzymes depended on the nature of the P1 residue.

Each evolved cleavase was individually assayed on all M15-L-P1 target polypeptides. In this assay, an individual cleavase clone was expressed and purified, and then incubated with each peptide substrate for 3 hours at 52° C. Six μM enzyme in 5 mM phosphate buffer at pH 8 were used. The UV absorbance of both product and starting material in the final reaction was measured on HPLC and converted to percentage of conversion (FIG. 14A). M15-L-P-AR exhibited poor cleavage efficiency with the set of seven Cleavase clones, but further directed evolution can be used to address this issue. Additionally, efficiency of cleavage reactions were assessed on peptide-DNA conjugates. In this assay, peptide substrates were modified to have an azide group at the C-terminal lysine that was linked to dibenzocyclooctyne (DBCO)-activated PEG12 linker connected with a DNA oligo. M15-L-P1-GAEIAGDVAGGK peptides were used (SEQ ID NO: 9), and for D and N as P1, the Gly residue at P2 position was replaced with Val. In FIG. 6B, the cleavage events were monitored by UREA-PAGE assay. It was found that the first selected modified cleavase (M15-L Z001) provided 100% cleavage for polypeptides with the following M15-L-labeled P1 residues: A, I, L, M, Q, V. Other selected modified cleavases provided 80-100% cleavage for polypeptides with the following groups of M15-L-labeled P1 residues: D, E; S,T; G; N; H,Y; F,W. A broad cleavage of a single labeled terminal amino acid from the polypeptide can be achieved by combining two or more dipeptide cleavases in a set. For example, as shown in FIG. 14A and FIG. 14B, a set of 7 selected dipeptide cleavases can provide broad activity for removal of almost all M15-L-labeled P1 residues from the polypeptide. Other cleavase combinations can be created to achieve a desired level of cleavage specificity, such as different sets of two, three, four or more enzymes.

### Example 8. Engineered Dipeptide Cleavases can Accommodate a Substrate Peptide Modified with a Nucleic Acid Tag

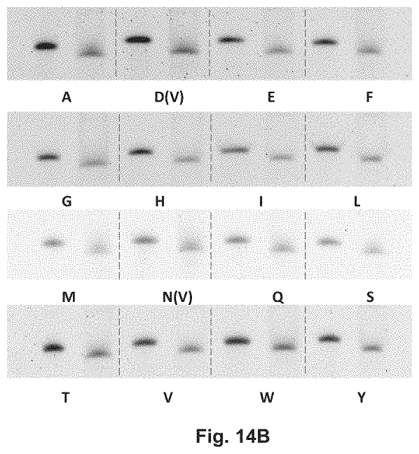

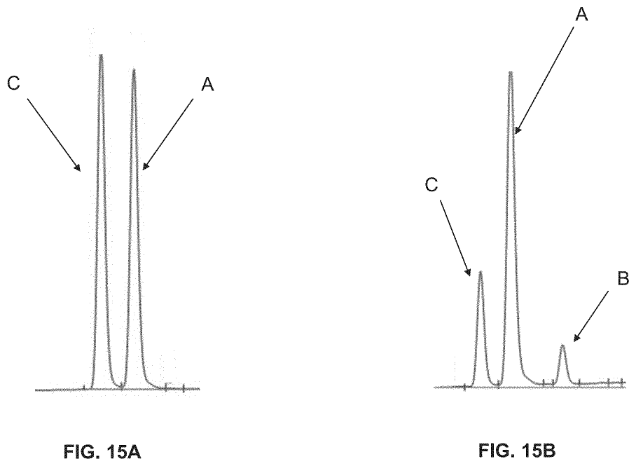

An exemplary cleavage reaction was performed to evaluate tolerance of a engineered dipeptidyl peptidase clone to a modified NTM group (M15-K(biotin)) attached to a model peptide AAR. An engineered dipeptidyl peptidase clone (SEQ ID NO: 11) derived from dipeptidyl peptidase from Thermomonas hydrothermalis (SEQ ID NO: 10) was expressed and purified, and then 2 uM of the engineered dipeptidyl peptidase was incubated with 300 uM of M15-K(biotin)-AAR (SEQ ID NO: 8) peptide for 2 hours at 52° C. in 5 mM phosphate buffer at pH 8. The UV absorbance at OD=254 nm of both starting material (FIG. 15A) and reaction product (FIG. 15B, after incubation with the peptidase) was measured on HPLC and converted to percentage of conversion. In FIG. 15A-FIG. 15B, A designates a signal from M15-K(biotin)-AAR peptide; B designates a signal from M15-K(biotin)-A molecule, and C designates a signal from a control peptide. After incubation with the peptidase for 1 h, about 10% of the model peptide was converted to the cleavage product, M15-K(biotin)-A (FIG. 15B). This indicates that an engineered dipeptide cleavase can be evolved to accommodate a substrate peptide modified a nucleic acid tag (biotin group can be substituted for a nucleic acid tag).

### Example 9. Engineering of the Cleavase Enzymes Capable of Cleaving the Modified NTAA of the Polypeptide

Cleavase engineering involves defining potential binding sites through rational, structure-based approaches on a parental scaffold and generating libraries that contain degenerate NNK codons at multiple, defined positions using Kunkel mutagenesis and phage display selection. Kunkel mutagenesis is a known site-directed mutagenesis strategy that introduces point mutations by annealing mutation-containing oligonucleotides to single-stranded uracil-containing single strand DNA (dU-ssDNA) templates. Exemplary Kunkel mutagenesis and phage display selection methods are described in U.S. Pat. No. 9,102,711 B2; U.S. Pat. No. 10,906,968 B2; Kunkel, Proc. Natl. Acad. Sci. USA, 1985, 83(2):488-492.

Cleavase maturation for specificity involved multiple cycles of error prone PCR prior to library construction via Kunkel mutagenesis and phage display selection. Briefly, 60-90 cycles of error prone PCR on a parental cleavase generated PCR amplicons with an average of 4-6 random amino acid mutations per 100 amino acids.

Cleavase enzyme expression and purification.

Plasmid DNA was generated containing the identified engineered cleavase enzyme conjugated with an N-terminal hexa-histidine tag. Plasmids were transformed into chemically competent E. coli cells using standard methods. Recovery was done by adding 150 ul of warm SOC and incubation for 1 hour at 30° C. After recovery, 80 ul of transformed culture was added to 1 ml 2YT containing corresponding antibiotic. The culture was grown overnight and then used to generate stock in glycerol. The stock was then used to inoculate an overnight culture of 2YT containing corresponding antibiotic, and the culture was grown overnight for ˜20 hours at 37° C. This culture was subsequently used to inoculate another larger volume culture of 2YT containing corresponding antibiotic at a 100-fold dilution. The culture was then left at 37° C. for 3-4 hours until an optical density of 0.6 was reached. Temperature was then lowered to 15° C. and protein expression was induced with a final concentration of 0.5 mM IPTG. The cultures were grown for an additional 16-20 hours and the cells were harvested by centrifugation at 4,000 rpm for 20 min. The cellular pellets were stored at −80° C. until ready for use.

Stored cellular pellets were resuspend in 25 mM Tris pH 7.9, 500 mM NaCl, and 10 mM imidazole with included protease inhibitor and were lysed by sonication. The clarified lysate was loaded onto an AKTA FPLC using a tandem purification method of nickel affinity and size-exclusion chromatography. The retained protein was eluted from the nickel affinity column using 25 mM Tris pH 7.9, 500 mM NaCl, 300 mM imidazole directly onto the size-exclusion column. The size-exclusion buffer was 25 mM PO4 pH 7.4 with 150 mM NaCl, and after elution and concentration, glycerol was added to final concentration of 10%. Proteins were aliquoted, frozen, and stored at −80° C.

### Example 10. Polypeptide Sample Preparation Workflow for the Encoding Assay

This example demonstrates an exemplary sample preparation workflow used for preparing peptide-recording tag conjugates and immobilizing them on a solid support.

Protein denaturation and digestion (see FIG. 4). For a 10 μg of protein sample, samples were diluted to the desired protein input concentration in an appropriate buffer (10 ug/45 μL; 100 mM carbonate/bicarbonate buffer at pH 9.15 with 0.1% sodium dodecyl sulfate (SDS)). Cysteines were reduced with TCEP added to a final concentration of 5 mM. Samples were incubated for 15 min at 37° C., and, after cooling, iodoacetamide (IAA) stock was added to a final concentration of 20 mM. Samples were incubated at 37° C. for 15 min to allow the alkylation to proceed. Lysine side chains were blocked by addition of NHS-acetate (ARR1, 10 mM) at 60° C. for 30 min. Trypsin was added at a 1:25 ratio, by mass, for each sample and incubated for 2 hours at 37° C. to digest the sample. Resulting peptides were then functionalized at the amine terminus using 10 mM photocleavable linker (AAR2, a self-immolative linker comprising para-nitrophenyl carbonate reactive ester coupled to a para-nitrobenzylcarbonate and an PEG-mTET enrichment tag) at 37° C. for 60 min.

Peptide immobilization to solid support (see FIG. 5). Peptides were immobilized to a solid support (TCO agarose, Click Chemistry Tools) through the enrichment tag (mTET moiety). The peptide mixture was incubated with 130 μL TCO beads for 60 min at 37° C. to immobilize the modified peptides. Other combinations of enrichment tag and compatible solid support can be implemented. Excess material (i.e. cellular components), unreacted peptides, and reaction components were removed by washing three times with PBS-T (PBS (phosphate-buffered saline) plus 0.1% TWEEN® 20).

CHD functionalization of C-terminal arginines and polypeptide-DNA conjugate formation (see FIG. 5). Each sample was resuspended after concentration in vacuo in 20 μL 0.2 M NaOH (pH 13.7), 1 M KPhos (pH 8.3), or 2 M KPhos (pH 8.3). CHD Stock (CHD-PEG₃-azide in DMSO) was added for a final concentration of 10 mM and incubated at 37° C. for 1 hr, 80° C. for 1.5 hours, or 80° C. for 1 hour, respectively. The reaction was neutralized by adding equal volume 1 M Tris, pH 7.4, and washed to remove excess/unreacted CHD-PEGS-azide and impurities. Samples were diluted to 10 μg/1000 μL in PBS-T. On-bead DNA-polypeptide conjugate (polypeptide—conjugation reagent—nucleic acid conjugate) formation was carried out using a solution of DBCO-DNA (Dibenzocyclooctyne-coupled DNA; DNA=5′-/5Phos/CAA GTT CTC AGT AAT GCG TAG/DBCOdT/CC GCG ACA CTA G-3; SEQ ID NO: 14) and incubating for 16 hours. The beads containing the conjugated product were washed to remove excess DBCO-DNA.

Further processing of peptide-DNA conjugates (see FIG. 5 and FIG. 6). Upon completion of incubation, beads were centrifuged and washed to remove any excess DBCO-DNA. Sample barcodes were added and beads were washed twice with 200 μL PBS-T. The peptide-DNA chimera was eluted with 10 μL 4 mM biotin, 20 mM Tris-HCl, and 50 mM NaCl. Chimera formation and barcoding were confirmed by loading 0.5 μL of sample (5 pmol) on TBU gel electrophoresis. (15% TBU gel, 200V, 50min). The peptides were then immobilized on a solid support. The DNA of the peptide-DNA chimera was hybridized and ligated to a DNA recording tag containing a complementary sequence attached to beads at appropriate spacing and density (see Example 3 of US 2022/0049246 A1, incorporated herein by reference).

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for identifying a polypeptide, the method comprising the steps of:
(a) providing the polypeptide and an associated recording tag joined to a solid support;
(b) contacting the polypeptide with a plurality of coding tags, wherein each coding tag of the plurality of coding tags is configured to react selectively with a specific type of amino acid residue(s) and comprises a barcode region with identifying information regarding the specific type of amino acid residue(s) to which the coding tag reacts selectively, thereby obtaining the polypeptide comprising coding tags attached to the specific amino acid residues;
(c) contacting the polypeptide comprising coding tags attached to the specific amino acid residues with a plurality of complementary coding tags, wherein each complementary coding tag of the plurality of complementary coding tags comprises (i) a region complementary to the barcode region of a corresponding coding tag, (ii) a first spacer region complementary to a first complementary spacer region of the recording tag, and (iii) a moiety configured, when in a close proximity, to be covalently coupled to the NTAA of the polypeptide, or to a modified NTAA of the polypeptide;
(d) providing conditions for covalently coupling the moiety to the NTAA of the polypeptide or the modified NTAA of the polypeptide;
(e) removing complementary coding tags that are not covalently coupled to the NTAA of the polypeptide;
(f) transferring identifying information of the barcode region or the region complementary to the barcode region from complementary coding tag covalently coupled to the NTAA of the polypeptide to the recording tag, wherein transferring the identifying information comprises a primer extension or ligation;
(g) removing the NTAA of the polypeptide, thereby exposing a new NTAA;
(h) adding a second order complementary spacer region to the recording tag extended at step (f);
(j) repeating steps (c)-(h) one or more times by replacing at step (c) the first spacer region of the complementary coding tags with a second or higher order spacer region complementary to the second or higher order complementary spacer region of the recording tag, and by replacing at step (h) the second complementary spacer region with a third or higher order complementary spacer region; and
(k) analyzing the recording tag extended at step (j) by a nucleic acid sequencing method, and obtaining information regarding the specific amino acid residues of the polypeptide, thereby identifying the polypeptide.

2. The method of claim 1, wherein at step (f) transferring information is performed by the primer extension using a DNA polymerase having a strand-displacement ability.

3. The method of claim 1, wherein at step (f) transferring information is performed by a splint ligation.

4. The method of claim 1, wherein the NTAA of the polypeptide is removed by an engineered enzyme.

5. The method of claim 1, wherein the NTAA of the polypeptide is removed by the following method:
(a) functionalizing the N-terminal amino acid (NTAA) of the polypeptide with a chemical reagent, wherein the chemical reagent is either:
(i) a compound of Formula (AA): 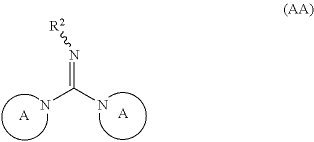
wherein: R2 is H or R4;
R4 is C1-6 alkyl, which is optionally substituted with one or two members selected from halo, C1-3 alkyl, C1-3 alkoxy, C1-3 haloalkyl, phenyl, 5-membered heteroaryl, and 6-membered heteroaryl, wherein the phenyl, 5-membered heteroaryl, and 6-membered heteroaryl are optionally substituted with one or two members selected from halo, —OH, C1-3 alkyl, C1-3 alkoxy, C1-3 haloalkyl, NO2, CN, COOR″, and CON(R″)2,
where each R″ is independently H or C1-3 alkyl;
each ring A is a 5-membered heteroaryl ring containing up to three N atoms as ring members and is optionally fused to an additional phenyl or a 5-6 membered heteroaryl ring, and wherein the 5-membered heteroaryl ring and optional fused phenyl or 5-6 membered heteroaryl ring are each optionally substituted with one or two groups selected from C1-4 alkyl, C1-4 alkoxy, —OH, halo, C1-4 haloalkyl, NO2, COOR, CONR2, —SO2R*, —NR2, phenyl, and 5-6 membered heteroaryl;
wherein each R is independently selected from H and C1-3 alkyl optionally substituted with OH, OR*, —NH2, —NHR*, or —NR*2; and
each R* is C1-3 alkyl, optionally substituted with OH, oxo, C1-2 alkoxy, or CN;
wherein two R, or two R″, or two R* on the same N can optionally be taken together to form a 4-7 membered heterocyclic ring, optionally containing an additional heteroatom selected from N, O and S as a ring member, and optionally substituted with one or two groups selected from halo, C1-2 alkyl, OH, oxo, C1-2 alkoxy, or CN; or
(ii) a compound of the formula R3-NCS;
wherein R3 is H or an optionally substituted group selected from phenyl, 5-membered heteroaryl, 6-membered heteroaryl, C1-3 haloalkyl, and C1-6 alkyl,
wherein the optional substituents are one to three members selected from halo, —OH, C₁₋₃ alkyl, C₁₋₃ alkoxy, C₁₋₃ haloalkyl, NO₂, CN, COOR′, —N(R′)₂, CON(R′)₂, phenyl, 5-membered heteroaryl, 6-membered heteroaryl, and C₁₋₆ alkyl, wherein the phenyl, 5-membered heteroaryl, 6-membered heteroaryl, and C₁₋₆ alkyl are each optionally substituted with one or two members selected from halo, —OH, C₁₋₃ alkyl, C₁₋₃ alkoxy, C₁₋₃ haloalkyl, NO₂, CN, COOR′, —N(R′)₂, and CON(R′)₂;
where each R′ is independently H or C₁₋₃ alkyl;
wherein two R′ on the same N can optionally be taken together to form a 4-7 membered heterocyclic ring, optionally containing an additional heteroatom selected from N, O and S as a ring member, and optionally substituted with one or two groups selected from halo, C₁₋₂ alkyl, OH, oxo, C₁₋₂ alkoxy, or CN;
to provide an initial NTAA functionalized polypeptide;
optionally treating the initial NTAA functionalized polypeptide with an amine of Formula R²—NH₂ or with a diheteronucleophile to form a secondary NTAA functionalized polypeptide; and
(b) treating the initial NTAA functionalized polypeptide or the secondary NTAA functionalized polypeptide with a suitable medium to eliminate the NTAA, thereby removing the NTAA of the polypeptide.

6. The method claim 5, wherein treating the initial NTAA functionalized polypeptide or the secondary NTAA functionalized polypeptide with the suitable medium occurs at temperature between about 40° C. and about 95° C.

7. The method of claim 1, wherein the moiety comprises a click chemistry reactive group.

8. The method of claim 1, which is for identifying 100 or more different polypeptides simultaneously.

9. The method of claim 1, wherein step (k) comprises bioinformatically matching the obtained information regarding the specific amino acid residues of the polypeptide with corresponding information extracted from a genomic database or a proteomic database.

10. The method of claim 1, wherein at step (a) the polypeptide is covalently attached to the associated recording tag.

11. The method of claim 1, wherein the specific type of amino acid residues is selected from the group consisting of: lysine, arginine, aspartate, glutamate, histidine, cysteine, serine, methionine, tryptophan and tyrosine.

12. The method of claim 1, wherein before contacting the polypeptide with the plurality of coding tags, the polypeptide is at least partially denatured to expose the specific amino acid residues to the plurality of coding tags.

13. A method for identifying a polypeptide, the method comprising the steps of:
(a) providing the polypeptide and an associated recording tag joined to a solid support;
(b) contacting the polypeptide with a plurality of coding tags, wherein each coding tag of the plurality of coding tags is configured to react selectively with a specific type of amino acid residue(s) and comprises: i) a barcode region with identifying information regarding the specific type of amino acid residue(s) to which the coding tag reacts selectively, ii) an identification region unique for each specific type of amino acid residue(s) to which the coding tags react selectively, and iii) a recognition region for a site-specific restriction enzyme, located between the barcode region and the identification region, thereby obtaining the polypeptide comprising coding tags attached to the specific amino acid residues;
(c) providing conditions for hybridization of the recording tag to one of the coding tags attached to the specific amino acid residue(s), thereby forming a double stranded region, and extending the recording tag, thereby transferring information of the barcode region and the recognition region from the coding tag to the recording tag;
(d) cutting the recognition region by providing the site-specific restriction enzyme, so that the extended recording tag is released and only a coding tag stub comprising the identification region of the coding tag remains attached to the polypeptide;
(e) repeating steps (c) and (d) for all other coding tags attached to the specific amino acid residues of the polypeptide, thereby obtaining the polypeptide comprising coding tag stubs attached to the specific amino acid residues;
(f) removing the NTAA of the polypeptide, thereby exposing a new NTAA;
(g) adding a second order complementary spacer region to the recording tag extended at step (f);
(h) restoring coding tags from the coding tag stubs;
(j) repeating steps (c)-(h) one or more times; and
(k) analyzing the recording tag extended at step (j) by a nucleic acid sequencing method, and obtaining information regarding the specific amino acid residues of the polypeptide before and after removing step, thereby identifying the polypeptide.

14. The method of claim 13, wherein the NTAA of the polypeptide is removed by an engineered enzyme.

15. The method of claim 13, wherein at step (a) the polypeptide is covalently attached to the associated recording tag.

16. The method of claim 13, wherein the specific type of amino acid residues is selected from the group consisting of: lysine, arginine, aspartate, glutamate, histidine, cysteine, serine, methionine, tryptophan and tyrosine.

17. A kit for identifying a polypeptide immobilized on a solid support, comprising:
(a) a plurality of coding tags, wherein each coding tag of the plurality of coding tags is configured to react selectively with a specific type of amino acid residue(s) from the polypeptide and comprises a barcode region with identifying information regarding the specific type of amino acid residue(s) to which the coding tag reacts selectively; and
(b) a plurality of complementary coding tags, wherein each complementary coding tag of the plurality of complementary coding tags comprises (i) a region complementary to the barcode region of a corresponding coding tag, (ii) a first spacer region complementary to a first complementary spacer region of a recording tag associated with the polypeptide, and (iii) a moiety configured, when in a close proximity, to be covalently coupled to the NTAA of the polypeptide, or to a modified NTAA of the polypeptide.

18. The kit of claim 17, wherein the moiety comprises a click chemistry reactive group.

19. The kit of claim 17, further comprising the solid support, wherein the recording tag is configured to be joined to the solid support.

20. The kit of claim 17, further comprising an engineered enzyme or a modifying reagent configured to remove the NTAA of the polypeptide.


SEQUENCE LISTING (14 sequences)

SEQ ID NO: 1 - DNA, 20 bp, synthetic construct
  source 1..20: mol_type = other DNA
  misc_feature 1..20: note = P5 primer
aatgatacggcgaccaccga

SEQ ID NO: 2 - DNA, 24 bp, synthetic construct
  source 1..24: mol_type = other DNA
  misc_feature 1..24: note = P7 primer
caagcagaagacggcatacgagat

SEQ ID NO: 3 - protein, 19 aa, synthetic construct
  REGION 1..19: note = FA-terminal peptide
FAGVAMPGAEDDVVGSGSK

SEQ ID NO: 4 - protein, 20 aa, synthetic construct
  REGION 1..20: note = AFA-terminal peptide
AFAGVAMPGAEDDVVGSGSK

SEQ ID NO: 5 - protein, 19 aa, synthetic construct
  REGION 1..19: note = AA-terminal peptide
AAGVAMPGAEDDVVGSGSK

SEQ ID NO: 6 - DNA, 33 bp, synthetic construct
  source 1..33: mol_type = other DNA
  misc_feature 1..33: note = Bait DNA
  misc_feature 1: note = 5'-phosphorylated
caagttctcagtaatgcgtagccgcgacactag

SEQ ID NO: 7 - DNA, 97 bp, synthetic construct
  source 1..97: mol_type = other DNA
  misc_feature 1..97: note = Capture DNA
  misc_difference 17..26: note = n is a, c, g, or t
cactcagtccattaacnnnnnnnnnnctagtgtcgcggactacgcattactgagaagcttgctagtcgacgtggtccttttggaccacgtcgactag

SEQ ID NO: 8 - protein, 4 aa, synthetic construct
  REGION 1..4: note = test peptide
  SITE 1: note = modified with M15-biotin
KAAR

SEQ ID NO: 9 - protein, 14 aa, synthetic construct
  REGION 1..14: note = test peptide
  VARIANT 2: note = X can be any amino acid
  SITE 1: note = modified with M15 label
LXGAEIAGDVAGGK

SEQ ID NO: 10 - protein, 723 aa, Thermomonas sp.
  REGION 1..723: note = Dipeptidyl peptidase from S46 family
MHKTRLVAALAAALATLAPAAWADEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDNWMWPRHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNNTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFDGTLESVASNWVFDPVLTRMISVDQRYMRWIMQEVMPAPQLLEELGVPPRQ

SEQ ID NO: 11 - protein, 700 aa, synthetic construct
  REGION 1..700: note = engineered dipeptidyl peptidase clone
DEGMWVPQQLPEIAGALKKAGLKLDPKQLSDLTGDPMGAVVSLGGCTGSFVSPQGLVATNHHCAYGAIQLNSTPEKNLIKDGFNAPTQADELSAGPNARIYVLEGITDVTAQAKAAMAAAGNDPVARANALEAFEKKITSDCEAEPGYRCRVYSFMGGITYRLFKNLEIKDVRLVYAPPSSVGKFGGDIDMGMWPTHTGDFSFYRAYVGKDGKPAPYSKDNVPYRPKHWLKIADTPLGEGDFVMVAGYPGRTDRYALVAEFENTQNWLYPAISKAYKDQIALVEAAAKDNPEIAVKYAAALAGWNRTSKNFDGQLEGFKRNDVLAIKRREEAAVLEWLRARGKAGTPALEAHAALVKLVADTARTQERDLVLGSFNRTGIIGVAVNLYRLAIERQKPDAEREPGYQQRDLPVIEGSLKQMERRYVPAMDRQLRAYWLDRYVALPAAQHVAAVDAWLGGSDKAAAEAALARLDQSRLGSLEERLKWFNADRAAFEASTDPAIQYAVAVMPTLLAMEQQAKTRYGVALEARPRYLQAVVDYKKSKGQAVYPDANSTLRITYGHVKGYTGLNGKVYTPFTTLEEVAAKETGVEPFDNPKALLEAVAAKRYAGLADARLGTVPVNFLADLDITGGNSGSPVLDANGRLVGLAFAVTLESVASNWVFDPVLTRMISVDQRYMRWIMQEVMPAPQLLEELGVPPRQ

SEQ ID NO: 12 - DNA, 18 bp, synthetic construct
  source 1..18: mol_type = other DNA
  misc_feature 1..18: note = splint DNA
ccattaacctagtgtcgc

SEQ ID NO: 13 - DNA, 16 bp, synthetic construct
  source 1..16: mol_type = other DNA
  misc_feature 1..16: note = barcode template
  misc_feature 1: note = 5'-phosphorylated
gttaatggactgagtg

SEQ ID NO: 14 - DNA, 34 bp, synthetic construct
  source 1..34: mol_type = other DNA
  misc_feature 1..34: note = DBCO-DNA
  misc_feature 1: note = 5'-phosphorylated
  misc_feature 22: note = modified with dibenzocyclooctyne (DBCO)
caagttctcagtaatgcgtagtccgcgacactag